(19) United States
(12) Patent Application Publication
(10) Pub. No.: US 2008/0287418 A1
(43) Pub. Date: Nov. 20, 2008

(54) Extended Release Compositions and Methods for Their Manufacture

(75) Inventors: Daniel Brown, Wilmington, DE (US); Donna Caster, Wilmington, DE (US); Brian Clark, Macclesfield (GB); Sandra Hopkins, Wilmington, DE (US); Jennifer Llewelyn, Wilmington, DE (US); Elizabeth Meehan, Macclesfield (GB); Lisa Martin, Wilmington, DE (US); Robert Timko, Wilmington, DE (US); Husheng Yang, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/939,585

(22) Filed: Nov. 14, 2007

Related U.S. Application Data
(60) Provisional application No. 60/930,643, filed on May 16, 2007.

(51) Int. Cl. A61K 31/554; A61P 25/18

(52) U.S. Cl. 514/211.13

(57) ABSTRACT

Extended release formulations of quetiapine and its pharmaceutically salts, and methods for manufacture of the formulations, may include the use of polymers selected for their physical and chemical characteristics. The formulations may include polymers selected to cause solid dosage forms of the formulations to conform to preselected quetiapine release criteria. The formulations may include non-polymer materials that may affect quetiapine release.

### CROSS-REFERENCE TO RELATED APPLICATION

This is a nonprovisional under 35 U.S.C. §119(e) of U.S. Provisional Application No. 60/930,643, filed on May 16, 2007, which is hereby incorporated herein by reference in its entirety.

### FIELD OF THE INVENTION

The present invention relates to a formulation of 11-[4-[2-(2-hydroxyethoxy)ethyl]-1-piperazinyl]dibenzo[b,f][1,4]thiazepine(quetiapine). More particularly, the invention relates to an extended release pharmaceutical composition comprising quetiapine or a pharmaceutically acceptable salt thereof.

2. Background

The compound 11-[4-[2-(2-hydroxyethoxy)ethyl]-1-piperazinyl]dibenzo[b,f][1,4]thiazepine (see Formula 1), having the common name “quetiapine,” and its pharmaceutically acceptable salts, exhibit useful antidopaminergic activity and may be used, for example, as an antipsychotic agent (for example, for the management of the manifestations of psychotic disorders) or as a treatment for hyperactivity. The compound may be used as an antipsychotic agent with a substantial reduction in the potential to cause side effects such as acute dystonia, acute dyskinesia, pseudo-Parkinsonism and tardive dyskinesia which side-effects may result from the use of typical antipsychotics or neuroleptics.

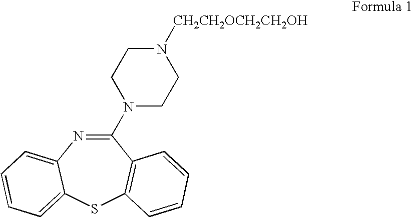

The preparation, physical properties and beneficial pharmacological properties of quetiapine, and its pharmaceutically acceptable salts are described in European Patents Nos. 240,228 and 282,236 and in U.S. Pat. No. 4,879,288, the contents of which are hereby incorporated herein by reference in their entireties.

It is desirable in the treatment of a number of diseases, both therapeutically and prophylactically, to provide an active pharmaceutical ingredient in an extended release form. Extended release may provide a generally uniform and constant rate of release over an extended period of time and may achieve a desired blood or blood plasma level of the active ingredient without the need for frequent administration of the ingredient.

While there are numerous extended release compositions known in the art that utilize gelling agents, such as hydroxypropyl methylcellulose (also referred to herein as “HPMC” and “hypromellose”), it has been found to be difficult to formulate extended release formulations of soluble medicaments and gelling agents, such as hypromellose, for several reasons. Principally, it has been found to be difficult to achieve the desired dissolution profiles or to control the rate of release of active ingredients that are soluble in aqueous media (as is the case for quetiapine, which is slightly soluble in water and soluble in acid). Among other issues, such active ingredients tend to generate an extended release product that is susceptible to a phenomenon known as dose dumping. That is, release of the active ingredient is delayed for a time but once release begins to occur the rate of release is very high. Moreover, fluctuations tend to occur in the plasma concentrations of the active ingredient, thus increasing the likelihood of toxicity. Further, some degree of diurnal variation in plasma concentration of the active ingredient has also been observed.

Because of the numerous physical and chemical interactions between constituents of some pharmaceutical compositions, it is also often difficult to combine the constituents in a manner that gives a formulation desirable physical or chemical characteristics.

Accordingly, it would be desirable to provide extended release formulations of water soluble medicaments, such as 11-[4-[2-(2-hydroxyethoxy)ethyl]-1-piperazinyl]dibenzo[b,f][1,4]thiazepine or a pharmaceutically acceptable salt that provide improved performance and may overcome, or at least alleviate, one or more of the above described difficulties.

### SUMMARY

Formulations including quetiapine and its pharmaceutically acceptable salts, and methods of making the formulations are provided.

A formulation may include a hydrophilic matrix comprising a gelling agent, 11-[4-[2-(2-hydroxyethoxy)ethyl]-1-piperazinyl]dibenzo[b,f][1,4]thiazepine, or a pharmaceutically acceptable salt thereof, such as a hemifumarate salt, and one or more pharmaceutically acceptable excipients.

Examples of gelling agents that may be present in the embodiments of the invention include such substances as hydroxypropylcellulose, hydroxymethylcellulose, hydroxyethylcellulose, hydroxypropyl ethylcellulose, methylcellulose, ethylcellulose, carboxyethylcellulose, carboxymethyl hydroxyethylcellulose, carbomer, sodium carboxymethylcellulose, polyvinylpyrrolidone, and the like, or mixtures thereof. In certain embodiments, the gelling agent can comprise hypromellose.

The amount of gelling agent, in combination with the quetiapine and any excipients, may be selected such that the active ingredient is released from the formulation, in a controlled fashion, over a period of about 24 hours.

The gelling agent may be present in a range that is about 5 to 50% (by weight). The range may be about 5 to 10%. The range may be about 20 to 50%. The range may be about 25 to 50%. The range may be 28 to 50%. The range may be 30 to 50%. (Weight percentages, as used herein, are relative to the core tablet weight, excluding the weight of any coating, unless otherwise specified.)

Some embodiments of the invention may include hypromellose mixtures that include more than one grade of polymer. Hypromellose polymers are commercially available under several trademarks, e.g. METHOCEL® E, F, J and K from the Dow Chemical Company, U.S.A. and METOLOSE™ 60SH, 65SH and 90SH from Shin-Etsu, Ltd., Japan. The grades may have differences in methoxy and hydroxypropoxy content as well as in viscosity and other characteristics. Different lots of hypromellose, even of the same grade may have differences in methoxy and hydroxypropoxy contents, viscosity and other characteristics.

The formulation may contain a buffer or pH modifier, for example if the active ingredient exhibits pH-dependent solubility, as is the case for quetiapine salts such as quetiapine fumarate.

The formulation will, in general, contain one or more excipients. Such excipients may include diluents such as lactose, microcrystalline cellulose, dextrose, mannitol, sucrose, sorbitol, gelatin, acacia, dicalcium phosphate, tricalcium phosphate, monocalcium phosphate, sodium phosphate, sodium carbonate and the like, preferably lactose and microcrystalline cellulose; lubricants such as stearic acid, zinc, calcium or magnesium stearate and the like, preferably magnesium stearate; binders such as sucrose, polyethylene glycol, povidone (polyvinylpyrrolidone), corn or maize starch, pregelatinized starch and the like; colorants such as ferric oxides, FD & C dyes, lakes and the like; flavoring agents; and pH modifiers that include suitable organic acids or alkali metal (e.g. lithium, sodium or potassium) salts thereof, such as benzoic acid, citric acid, tartaric acid, succinic acid, adipic acid and the like or the corresponding alkali metal salts thereof, preferably the alkali metal salts of such acids and in particular the sodium salt of citric acid (i.e. sodium citrate). As is well known, some excipients have multiple functions, such as being both a diluents and a binder.

In some embodiments of the invention, the formulation may be present in a solid dosage form such as a tablet, caplet or any other suitable form comprising 11-[4-[2-(2-hydroxyethoxy)ethyl]-1-piperazinyl]dibenzo[b,f][1,4]thiazepine hemifumarate (“quetiapine fumarate”), 6-18% by weight sodium citrate dihydrate, 30.0% by weight hydroxypropyl methylcellulose, wherein 15-29 of the 30.0% is a first hydroxypropyl methylcellulose constituent; the remainder of the 30.0% is a second hydroxypropyl methylcellulose constituent; and the first and second constituents correspond, respectively, to a first hydroxypropyl methylcellulose grade that has an “apparent viscosity” (see below) between 80 centipoise (“cp”) and 120 cp and a second hydroxypropyl methylcellulose that has a apparent viscosity between 3000 cp and 5600 cp. The tablet may comprise 11-12% by weightll-[4-[2-(2-hydroxyethoxy)ethyl]-1-piperazinyl]dibenzo[b,f][1,4]thiazepine hemifumarate. The tablet may comprise 29.5-30.5% by weight 11-[4-[2-(2-hydroxyethoxy)ethyl]-1-piperazinyl]dibenzo[b,f][1,4]thiazepine hemifumarate. The tablet may comprise 37.9-38.9% by weight 11-[4-[2-(2-hydroxyethoxy)ethyl]-1-piperazinyl]dibenzo[b,f][1,4]thiazepine hemifumarate. In some embodiments, the tablet comprises 52.4-53.4% by weight 11-[4-[2-(2-hydroxyethoxy)ethyl]-1-piperazinyl]dibenzo[b,f][1,4]thiazepine hemifumarate.

In some embodiments, the viscosities of the hydroxypropyl methylcellulose are consistent with Ubbelohde viscometer apparent viscosities of 2% by weight hydroxypropyl methylcellulose in 20° water, as determined using the method described in The United States Pharmacopoeia (USP30-NF25), United States Pharmacopoeia Convention, Inc. 2007, p. 2323.

In some embodiments of the invention, the formulation comprises sodium citrate dihydrate present in about 7.2-12.5% by weight. In some embodiments, the formulation comprises sodium citrate dihydrate present in 7.2% by weight. In some embodiments, the formulation comprises sodium citrate dihydrate present in 11.5% by weight. In some embodiments, the formulation comprises sodium citrate dihydrate present in 12.5% by weight.

In some embodiments of the invention, the formulation comprises lactose monohydrate present in up to about 30% by weight. In some embodiments, the formulation comprises lactose monohydrate present in 25.1% by weight. In some embodiments, the formulation comprises lactose monohydrate present in 13.0% by weight. In some embodiments, the formulation comprises lactose monohydrate present in 8.8% by weight. In some embodiments, the formulation comprises lactose monohydrate present in 1.8% by weight.

In some embodiments, the formulation comprises microcrystalline cellulose present in up to about 30% by weight. In some embodiments, the formulation comprises microcrystalline cellulose present in 25.1% by weight. In some embodiments, the formulation comprises microcrystalline cellulose present in 13.0% by weight. In some embodiments, the formulation comprises microcrystalline cellulose present in 8.8% by weight. In some embodiments, the formulation comprises microcrystalline cellulose present in 1.8% by weight.

In some embodiments, the tablet comprises an amount of magnesium stearate between about 1% and 3% by weight. In some embodiments, the tablet comprises magnesium stearate present in 1.0% by weight. In some embodiments, the tablet comprises magnesium stearate present in 1.5% by weight. In some embodiments, the tablet comprises magnesium stearate present in 2.0% by weight.

In some embodiments, the hydroxypropyl methylcellulose comprises 9.8 to 13.4% by weight of the hydroxypropyl methylcellulose, as measured by nuclear magnetic resonance (“NMR”), hydroxypropoxy. In some embodiments, the hydroxypropyl methylcellulose comprises 26.4 to 29.2% by weight of the hydroxypropyl methylcellulose, as measured by NMR, methoxy.

In some embodiments of the invention, the solid dosage form comprises 50 milligram (“mg”) quetiapine, for example in a 500 mg total core mass. In some embodiments, the solid dosage form comprises 150 mg quetiapine, for example, in a 575 mg total core mass. In some embodiments, the solid dosage comprises 200 mg quetiapine, for example in a 600 mg total core mass. In some embodiments, the solid dosage form comprises 400 mg quetiapine, for example in an 870 mg total core mass.

In some embodiments of the invention, the formulation is present in a solid dosage form comprising 50 mg quetiapine, the dosage form, after ingestion under steady state conditions by a human, resulting in a blood plasma concentration, in nanograms quetiapine per milliliter plasma, that is up to about: 67.6 at 1 hour after the ingestion; 124 at 4 hours after the ingestion; 105 at 8 hours after the ingestion; 74.3 at 12 hours after the ingestion; and 236 at 16 hours after the ingestion.

In some embodiments of the invention, the formulation is a solid dosage form comprising 200 mg quetiapine, the dosage form, after ingestion under steady state conditions by a human, resulting in a blood plasma concentration, in nanograms quetiapine per milliliter plasma, that is: up to about 251 at 1 hour after the ingestion; between about 32.2 and about 416 at 4 hours after the ingestion; up to about 496 at 8 hours after the ingestion; between about 4.6 and about 323 at 12 hours after the ingestion; and up to about 251 at 16 hours after the ingestion.

In some embodiments of the invention, the formulation is a solid dosage form comprising 400 mg quetiapine, the dosage form, after ingestion under steady state conditions by a human, resulting in a blood plasma concentration, in nanograms quetiapine per milliliter plasma, that is: between about 15.9 and about 391 at 1 hour after the ingestion; up to about 1052 at 4 hours after the ingestion; between about 63.1 and about 785 at 8 hours after the ingestion; between about 11.1 and about 613 at 12 hours after the ingestion; and up to about 448 at 16 hours after the ingestion.

In some embodiments of the invention, a dosage form comprises: 30.0% by weight hydroxypropyl methylcellulose and 7.2% by weight sodium citrate dihydrate. In certain embodiments, 15-29 of the 30.0% is a first hydroxypropyl methylcellulose constituent; the remainder of the 30.0% is a second hydroxypropyl methylcellulose constituent; and the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp. In some embodiments, the viscosities of the dosage form are consistent with Ubbelohde viscometer apparent viscosities of 2% by weight hydroxypropyl methylcellulose in 20° water, as determined using the method described in The United States Pharmacopoeia (USP30-NF25), United States Pharmacopoeia Convention, Inc. 2007, p. 2323. In some embodiments, the first and second constituents, respectively, have viscosities of 80-120 cp and 3000-5600 cp.

In some embodiments of the invention, a solid dosage form comprises 50 mg quetiapine, the dosage form, after ingestion under steady state conditions by a human, resulting in a time-dependent blood plasma quetiapine concentration, in nanograms quetiapine per milliliter plasma, having a maximum value, Cₘₐₓ, that is up to about 239 and corresponds to a time tₘₐₓ that is between 2 and 16 hours after the ingestion. In some embodiments, the concentration has a C₂₄ value, that is up to about 39.2 and corresponds to a time t₂₄, at 24 hours after the ingestion; and the ratio Cₘₐₓ:C₂₄ is up to about 35.2.

In some embodiments of the invention, a solid dosage form comprises 200 mg quetiapine, the dosage form, after ingestion under steady state conditions by a human, resulting in a time-dependent blood plasma quetiapine concentration, in nanograms quetiapine per milliliter plasma, having a maximm value, Cₘₐₓ, that is between about 3.9 and about 601 and corresponds to a time tₘₐₓ that is between 2 and 8 hours after the ingestion. In some embodiments, the concentration has a C₂₄ value that is up to about 156 and corresponds to a time t₂₄, at 24 hours after the ingestion; and the ratio Cₘₐₓ:C₂₄ is up to about 20.9.

In some embodiments of the invention, a solid dosage form comprises 400 mg quetiapine, the dosage form, after ingestion under steady state conditions by a human, resulting in a time-dependent blood plasma quetiapine concentration, in nanograms quetiapine per milliliter plasma, having a maximum value, Cₘₐₓ, that is between about 80 and about 1109 and corresponds to a time tₘₐₓ that is between 3 and 8 hours after the ingestion. In some embodiments, the concentration has a C₂₄ value that is up to about 265 and corresponds to a time t₂₄, at 24 hours after the ingestion; and the ratio Cₘₐₓ:C₂₄ is up to about 25.9.

In some embodiments of the invention, a solid dosage form comprises 50 mg quetiapine, the dosage form, after ingestion under steady state conditions by different humans, resulting in distinct time-dependent blood plasma quetiapine concentrations, which have a maximum value C_{ave,max} between about 5.1 and about 117 nanograms quetiapine per milliliter plasma, C_{ave,max} corresponding to a time that is between 2.5 and 3.5 hours after ingestion. In some embodiments, the distinct concentrations have an average value C_{ave,24} that is about 14.8 and corresponds to a time 24 hours after the ingestion; and the ratio C_{ave,max}:C_{ave,24} is about 4.1.

In some embodiments of the invention, a solid dosage form comprises 200 mg quetiapine, the dosage form, after ingestion under steady state conditions by different humans, resulting in distinct time-dependent blood plasma quetiapine concentrations, which have a maximum value C_{ave,max} that is up to about 550.4 nanograms quetiapine per milliliter plasma, C_{ave,max} corresponding to a time that is between 5.5 and 6.5 hours after ingestion. In some embodiments, the distinct concentrations have an average value C_{ave,24} that is about 64.9 and corresponds to a time 24 hours after the ingestion; and the ratio C_{ave,max}:C_{ave,24} is about 4.0.

In some embodiments of the invention, a solid dosage form comprises 400 mg quetiapine, the dosage form, after ingestion under steady state conditions by different humans, resulting in distinct time-dependent blood plasma quetiapine concentrations, which have a maximum value C_{ave,max} that is up to about 1062 nanograms quetiapine per milliliter plasma, C_{ave,max} corresponding to a time that is between 2.5 and 3.5 hours after ingestion. In some embodiments, the distinct concentrations have an average value C_{ave,24} that is about 114 and corresponds to a time 24 hours after the ingestion; and the ratio C_{ave,max}:C_{ave,24} is about 4.6.

In some embodiments of the invention, a solid dosage form comprises 50 mg quetiapine, the dosage form, after ingestion under steady state conditions by different humans, resulting in distinct time-dependent blood plasma quetiapine concentrations, which have a cumulative area-under-the-curve, AUC_{cum}, that is: up to 46 at 1 hour after ingestion; between 8 and 352 at 4 hours after ingestion; between 34 and 789 at 8 hours after ingestion; between 83 and 1092 at 12 hours after ingestion; between 111 and 1396 at 16 hours after ingestion; and up to 1935 at 24 hours after ingestion; wherein AUC_{cum} has units of (nanogram quetiapine)×hour/milliliter.

In some embodiments of the invention, a solid dosage form comprises 200 mg quetiapine, the dosage form, after ingestion under steady state conditions by different humans, resulting in distinct time-dependent blood plasma quetiapine concentrations, which have a cumulative area-under-the-curve, AUC_{cum}, that is:up to 177 at 1 hour after ingestion; between 35 and 1318 at 4 hours after ingestion; between 188 and 3115 at 8 hours after ingestion; between 251 and 4650 at 12 hours after ingestion; between 362 and 5666 at 16 hours after ingestion; and between 441 and 6899 at 24 hours after ingestion; wherein AUC_{cum} has units of (nanogram quetiapine)×hour/milliliter.

In some embodiments of the invention, a solid dosage form comprises 400 mg quetiapine, the dosage form, after ingestion under steady state conditions by different humans, resulting in distinct time-dependent blood plasma quetiapine concentrations, which have a cumulative area-under-the-curve, AUC_{cum}, that is between: 3 and 320 at 1 hour after ingestion; 143 and 2677 at 4 hours after ingestion; 575 and 6158 at 8 hours after ingestion; 916 and 8722 at 12 hours after ingestion; 1037 and 10685 at 16 hours after ingestion; 1031 and 13033; and 1031 and 13033 at 24 hours after ingestion; wherein AUC_{cum} has units of (nanogram quetiapine)×hour/milliliter.

In some embodiments of the invention, a formulation comprises quetiapine fumarate and 30.0% hydroxypropyl methylcellulose, wherein 15-29 of the 30.0% is a first hydroxypropyl methylcellulose constituent, such that the formulation satisfies a predetermined dissolution criterion; the remainder of the 30.0% is a second hydroxypropyl methylcellulose constituent; the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp.

In some embodiments, the formulation comprises 11-12% by weight quetiapine fumarate. In some embodiments, the formulation comprises 29.5-30.5% by weight quetiapine fumarate. In some embodiments, the formulation comprises 37.9-38.9% by weight quetiapine fumarate. In some embodiments, the formulation comprises 52.4-53.4% by weight quetiapine fumarate.

In some embodiments, the formulation comprises quetiapine or a pharmaceutically acceptable salt thereof wherein the quetiapine content is about 9.6% to about 10.4% by weight and wherein the formulation comprises about 30% hydroxypropyl methylcellulose by weight and about 7.2% sodium citrate dihydrate by weight.

In some embodiments, the formulation comprises quetiapine or a pharmaceutically acceptable salt thereof wherein the quetiapine content is about 25.6 to about 26.5% by weight and wherein the dosage form comprises about 30% hydroxypropyl methylcellulose by weight and about 12.5% sodium citrate dihydrate by weight.

In some embodiments, the formulation comprises quetiapine or a pharmaceutically acceptable salt thereof wherein the quetiapine content is about 32.9% to about 33.8% by weightand wherein the dosage form comprises about 12.5% sodium citrate dihydrate by weight and about 30% hydroxypropyl methylcellulose by weight.

In some embodiments, the formulation comprises quetiapine or a pharmaceutically acceptable salt thereof wherein the quetiapine content is about 37.1% to about 38.0% by weight and wherein the dosage form comprises about 12.5% sodium citrate dihydrate by weightand about 30% hydroxypropyl methylcellulose by weight and wherein about 15 to about 29 of the 30% hydroxypropyl methylcellulose is a first hydroxypropyl methylcellulose constituent; the remainder of the 30% is a second hydroxypropyl methylcellulose constituent; and the first and second constituents correspond, respectively, to a first hydroxypropyl methylcellulose grade that has a apparent viscosity between about 80 cp and about 120 cp and a second hydroxypropyl methylcellulose that has an apparent viscosity between about 3000 cp and about 5600 cp, wherein the ratio of the first hydroxypropyl methylcellulose grade to the second hydroxypropyl methylcellulose grade is not 25.0 to 5.0 In some embodiments, the formulation comprises quetiapine or a pharmaceutically acceptable salt thereof wherein the quetiapine content is about 45.5% to about 46.4% by weight and wherein the dosage form comprises about 11.5% sodium citrate dihydrate by weight and about 30% hydroxypropyl methylcellulose by weight.

In some embodiments, the invention comprises a method of effectively treating psychoses in humans, comprising orally administering to a human patient on a once-a-day basis an oral extended release dosage form containing quetiapine or a pharmaceutically acceptable salt thereof wherein the quetiapine content is 50 mg which at steady-state provides a time to maximum plasma concentration (tₘₐₓ) of said antipsychotic in about 2 to about 16 hours, a maximum plasma concentration (Cₘₐₓ) which is greater than or equal to four times the plasma concentration of said antipsychotic at about 24 hours, and which dosage form provides effective treatment of psychoses for about 24 hours or more after administration to the patient.

In some embodiments, the invention comprises a method of effectively treating psychoses in humans, comprising orally administering to a human patient on a once-a-day basis an oral extended release dosage form containing quetiapine or a pharmaceutically acceptable salt thereof wherein the quetiapine content is 150 mg which at steady-state provides a time to maximum plasma concentration (tₘₐₓ) of said antipsychotic in about 2 to about 16 hours, a maximum plasma concentration (Cₘₐₓ) which is greater than or equal to four times the plasma concentration of said antipsychotic at about 24 hours, and which dosage form provides effective treatment of psychoses for about 24 hours or more after administration to the patient.

In some embodiments, the invention comprises a method of effectively treating psychoses in humans, comprising orally administering to a human patient on a once-a-day basis an oral extended release dosage form containing quetiapine or a pharmaceutically acceptable salt thereof wherein the quetiapine content is 200 mg which at steady-state provides a time to maximum plasma concentration (tₘₐₓ) of said antipsychotic in about 2 to about 8 hours, a maximum plasma concentration (Cₘₐₓ) which is greater than or equal to four times the plasma concentration of said antipsychotic at about 24 hours, and which dosage form provides effective treatment of psychoses for about 24 hours or more after administration to the patient.

In some embodiments, the invention comprises a method of effectively treating psychoses in humans, comprising orally administering to a human patient on a once-a-day basis an oral extended release dosage form containing quetiapine or a pharmaceutically acceptable salt thereof wherein the quetiapine content is 400 mg which at steady-state provides a time to maximum plasma concentration (tₘₐₓ) of said antipsychotic in about 3 to about 8 hours, a maximum plasma concentration (Cₘₐₓ) which is greater than or equal to four times the plasma concentration of said antipsychotic at about 24 hours, and an area under curve between the time of administration and 24 hours after administration (AUC_{cum, 24}) which is greater than or equal to about 6000 ng.hr/mL, and which dosage form provides effective treatment of psychoses for about 24 hours or more after administration to the patient.

In some embodiments, when dissolution of the formulation takes place in a basket apparatus having a rotation speed of 200 revolutions per minute and containing 900 milliliter 0.05 molar sodium citrate and 0.09 normal sodium hydroxide, to which 100 milliliter 0.05 molar sodium phosphate and 0.46 normal sodium hydroxide are added after 5 hours: no more than 20% of the quetiapine is dissolved during the first one-hour period of the dissolution. In some embodiments, 47-69% of the quetiapine is dissolved during the first 6-hour period of the dissolution. In some embodiments, 65-95% of the quetiapine is dissolved during the first 12-hour period of the dissolution. In some embodiments, at least 85% of the quetiapine is dissolved during the first 20-hour period of the dissolution.

In some embodiments of the invention, a formulation comprises quetiapine fumarate and 30.0% hydroxypropyl methylcellulose, wherein 15-29 of the 30.0% is a first hydroxypropyl methylcellulose constituent, such that the formulation optimally exhibits at least one dissolution target; the remainder of the 30.0% is a second hydroxypropyl methylcellulose constituent; the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp.

In some embodiments, the formulation comprises 11-12% by weight quetiapine fumarate. In some embodiments, the formulation comprises 29.5-30.5% by weight quetiapine fumarate. In some embodiments, the formulation comprises 37.9-38.9% by weight quetiapine fumarate. In some embodiments, the formulation comprises 52.4-53.4% by weight quetiapine fumarate.

In some embodiments, a first target is, when dissolution takes place in a basket apparatus having a rotation speed of 200 revolutions per minute and containing 900 milliliter 0.05 molar sodium citrate and 0.09 normal sodium hydroxide, to which 100 milliliter 0.05 molar sodium phosphate and 0.46 normal sodium hydroxide are added after 5 hours: 58% of the quetiapine is dissolved in the first six-hour period of the dissolution. In some embodiments, a second target is: 80% of the quetiapine is dissolved in the first 12-hour period of the dissolution.

In some embodiments of the invention, a solid dosage form comprises a dose of quetiapine, the dosage form, after ingestion under steady state conditions by different humans, resulting in time-dependent blood plasma quetiapine concentrations, the average of which have a dose-scaled concentration, C/dose, that is between: about 0.433 and about 0.678 at 1 hour after administration; about 1.01 and about 1.35 at 4 hours after administration; about 0.930 and about 1.35 at 8 hours after administration; about 0.590 and about 1.07 at 12 hours after administration; and about 0.204 and about 1.22 at 16 hours after administration; wherein the dose is between 49.5 mg and 249.5 mg and C is expressed in nanogram quetiapine per milliliter plasma.

In some embodiments of the invention, a solid dosage form comprises a dose of quetiapine, the dosage form, after ingestion under steady state conditions by different humans, resulting in time-dependent blood plasma quetiapine concentrations, the average of which have a dose-scaled concentration, C/dose, that is between: about 0.433 and about 0.678 at 1 hour after administration; about 1.01 and about 1.35 at 4 hours after administration; about 0.930 and about 1.35 at 8 hours after administration; about 0.590 and about 1.07 at 12 hours after administration; and about 0.204 and about 1.22 at 16 hours after administration; wherein the dose is greater than 350 mg and C is expressed in nanogram quetiapine per milliliter plasma.

In some embodiments of the invention, a solid dosage form comprises an amount of quetiapine and 30.0% hydroxypropyl methylcellulose, wherein 15-29 of the 30.0% is a first hydroxypropyl methylcellulose constituent, such that the formulation optimally exhibits the time-dependent ratio C dose; the remainder of the 30.0% is a second hydroxypropyl methylcellulose constituent; the first and second constituents correspond, respectively, to a first hydroxypropyl methylcellulose grade that has an apparent viscosity between 80 cp and 120 cp and a second hydroxypropyl methylcellulose that has an apparent viscosity between 3000 cp and 5600 cp; and C dose is within a range defined by

$$base + \frac{exp ( - {K}_{a} \times t ) - exp ( - {K}_{e} \times t )}{{K}_{e} / {K}_{a} - 1.5} ,$$

in which: C is the average quetiapine blood plasma concentration, in nanogram quetiapine per milliliter plasma, at time t after administration of the quetiapine to a human; base is between, inclusively, 0.1227 and 0.2428; Kₑ is between, inclusively, 0.2344 and 0.2678; Kₐ is between, inclusively, 0.1396 and 0.1592; and the dose is between 49.5 mg and 249.5 mg.

In some embodiments, a solid dosage form comprises an amount of quetiapine and 30.0% hydroxypropyl methylcellulose, wherein 15-29 of the 30.0% is a first hydroxypropyl methylcellulose constituent, such that the formulation optimally exhibits a time-dependent ratio C:dose; the remainder of the 30.0% is a second hydroxypropyl methylcellulose constituent; the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp; and C:dose is within a range defined by

$$base + \frac{exp ( - {K}_{a} \times t ) - exp ( - {K}_{e} \times t )}{{K}_{e} / {K}_{a} - 1.5} ,$$

in which: C is the average quetiapine blood plasma concentration, in nanogram quetiapine per milliliter plasma, at time t after administration of the quetiapine to a human; base is between, inclusively, 0.1227 and 0.2428; Kₑ is between, inclusively, 0.2344 and 0.2678; Kₐ is between, inclusively, 0.1396 and 0.1592; and the dose is greater than 350 mg.

The invention may include a method for manufacturing a solid dose form having a composition that includes an active ingredient and first and second constituents. The active ingredient may be quetiapine. In some embodiments of the invention, the method may comprise inputting into a multivariate model first data corresponding to a first constituent; inputting into the model second data corresponding to a second constituent; using the model, identifying a ratio between a first constituent amount and a second constituent amount such that the dosage form satisfies a dissolution criterion when the composition includes the first and second constituents in proportion to the ratio. This method may be used, for example, to find a constituent ratio to obtain a desired dissolution profile in the face of variations in constituent properties, such as lot-to-lot or source-to-source variations, that may occur during the dosage form manufacture, such as commercial scale manufacture over an extended period of time, such as when identifcal constituent lots may not be readily available.

In some embodiments, the first and second constituents comprise, respectively, first and second hydroxypropyl methylcellulose lots. In some embodiments, the first and second lots have first and second viscosities, respectively, and the first viscosity is different from the second viscosity. In some embodiments, the first viscosity is in the range 80-120 cp, and the second viscosity is in the range 3000-5600 cp.

In some embodiments, the first and second data comprise measured viscosities corresponding to the first and second lots, respectively. In some embodiments, the first and second data comprise hydroxypropoxy contents of the first and second lots, respectively. In some embodiments, at least one of the hydroxypropoxy contents is measured using nuclear magnetic resonance. In some embodiments, at least one of the methoxy contents is measured using nuclear magnetic resonance.

In some embodiments, the first and second data comprise weight average molecular weights (hereinafter, “molecular weight” or “molecular weights,” as appropriate) corresponding to the first and second lots, respectively.

In some embodiments, the first and second data comprise methoxy contents of the first and second lots, respectively.

In some embodiments, the first and second data comprise particle size information corresponding to the first and second lots, respectively. Particle size information may be characterized as, for example, %-through-100-mesh (an index that may be taken from the supplier's certificate of analysis; smaller sieve “mesh” sizes of 3½ to 400 are designated by the number of openings per linear inch in the sieve. Thus, a 100 mesh sieve has 100 openings per inch. For example, a 100 mesh sieve may have holes that are 149×149 microns. % through a 100 mesh sieve is therefore the percentage by weight of particles that are less than 149 microns in diameter.). Particle size may also be characterized as median particle diameter (D50) and/or particle size span, both of which may be determined using a laser diffraction technique.

In some embodiments, the first and second data comprise number average molecular weight (hereinafter, “molecular number”) information corresponding to the first and second lots, respectively.

In some embodiments, the method comprises inputting into the model a quetiapine salt content corresponding to the composition.

In some embodiments, the method comprises inputting into the model an excipient content corresponding to the composition.

In some embodiments, the method comprises inputting the dosage form weight into the model.

In some embodiments, the method comprises inputting into the model a quetiapine amount corresponding to the composition; wherein the first and second data comprise, with respect to the first and second lots, respectively: hydroxypropoxy contents; and molecular weight information. In some embodiments, the hydroxypropoxy contents are characterized as weight percentages of a total hydroxypropyl methylcellulose weight.

In some embodiments, the ratio of the first to the second component has: a minimum value of 15% composition weight:15% composition weight; and a maximum value of 29% composition weight:1% composition weight.

In some embodiments, the dissolution criterion is satisfied when the formulation in a solid dosage form, when subjected to predetermined conditions for a time, dissolves to an extent that is within a predetermined range. In some embodiments, the dissolution criterion is satisfied when the extent is optimal within the range.

In some embodiments, when the ratio is a first ratio, using the model includes predicting dissolution for a second ratio; and the dissolution extent is optimal when the extent is closer to the center of the range than is the dissolution corresponding to the second ratio.

The invention may include a method for manufacturing a dosage form by establishing for first and second properties of first and second constituents, respectively, a correlation between a ratio and dissolution profile information; wherein the ratio is between a first constituent amount and a second constituent amount such that the dosage form satisfies a dissolution criterion when the composition includes the first and second constituents in proportion to the ratio.

In some embodiments, the first property promotes dissolution; and the second property retards dissolution. In some embodiments, the first property corresponds to hydroxypropoxy content.

In some embodiments, the second property corresponds to viscosity, molecular weight, or molecular number.

In some embodiments, the first property corresponds to hydroxypropoxy content and the second property corresponds to viscosity.

In some embodiments, the dissolution profile information includes a first value corresponding to a time and a second value corresponding dissolution extent at the time.

In some embodiments, the correlation may be embodied in a multivariate model.

The method may include measuring the hydroxypropoxy and methoxy of a plurality of batches of hydroxypropyl methylcellulose. In some embodiments the measuring is implemented using nuclear magnetic resonance (NMR). A first grade of the hypromellose has a first viscosity and a second grade may have a second viscosity. The method may include inputting into a multivariate model the tablet strength and the hydroxypropoxy content and molecular weight of each of the first grade and the second grade. The method may also include inputting into the model a series of ratios between an amount of the first grade and an amount of the second grade. The method may also include identifying, using the model, an optimum ratio that corresponds to a predicted dissolution profile that has a smaller deviation from a target profile than the deviation obtained using the other ratios. Alternatively, the method may include identifying, using the model, at least one ratio that produces a formulation that satisfies a desired dissolution profile.

In some embodiments, the model may be an artificial neural network (“ANN”) model.

In some embodiments, the correlation may be embodied in a look-up table.

### BRIEF DESCRIPTION OF THE FIGURES

The above and other features of the present invention, its nature and various advantages will be more apparent upon consideration of the following detailed description, taken in conjunction with the accompanying drawings, and in which:

FIG. 1 is a schematic diagram showing chemical structures that may be used in accordance with the principles of the invention.

FIG. 2 is a flow diagram showing a manufacturing process that may be used in accordance with the principles of the invention.

FIG. 3 is a graph showing clinical data based on a formulation in accordance with the principles of the invention.

FIG. 4 is a graph showing clinical data based on a formulation in accordance with the principles of the invention.

FIG. 5 is a graph showing clinical data based on a formulation that may be obtained using methods in accordance with the principles of the invention.

FIG. 6 is a graph showing clinical data based on a formulation in accordance with the principles of the invention.

FIG. 7 is a graph is a graph showing normalized clinical data from FIGS. 3-6.

FIG. 8 is a chart showing the affect of different factors on a property of a formulation in accordance with the principles of the invention.

FIG. 9 is a graph showing a correlation between an polymer chemical attribute and a polymer characteristic.

FIG. 10 is a graph showing a correlation between an polymer physical attribute and a polymer characteristic.

FIG. 11 is a graph showing in vitro dissolution data based on formulations in accordance with the principles of the invention.

FIG. 12 is a graph showing a characteristic of a gelling agent that may be used in accordance with the principles of the invention.

FIG. 13 is a graph showing the release of hypromellose for different grades of hypromellose that may be used in accordance with the principles of the invention.

FIG. 14 is a graph showing the release of hypromellose and a drug that may be used in accordance with the principles of the invention.

FIG. 15 is a schematic diagram showing the architecture of a multivariate model that may be used in accordance with the principles of the invention.

FIG. 16 is a schematic diagram of a multivariate model in accordance with the principles of the invention.

FIG. 17 is a graph showing predictive data and acceptance criteria in accordance with the principles of the invention.

FIG. 18 is a flow diagram showing a method of using the FIG. 15 model.

FIG. 19 is a flow diagram showing a method of using the FIG. 15 model.

FIG. 20 is an illustrative data table in accordance with the principles of the invention.

FIG. 21 is a graph of in vitro dissolution data based on a formulation in accordance with the principles of the invention.

FIG. 22 is a graph of in vitro dissolution data based on a formulation in accordance with the principles of the invention.

FIG. 23 is a graph of in vitro dissolution data based on a formulation in accordance with the principles of the invention.

FIG. 24 is a graph of in vitro dissolution data based on a formulation in accordance with the principles of the invention.

FIG. 25 is a graph of in vitro dissolution data based on a formulation in accordance with the principles of the invention.

### DETAILED DESCRIPTION OF THE EMBODIMENTS

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

In order to further define the invention, the following terms and definitions are provided herein.

The term “treating” or “treatment” is intended to include but is not limited to mitigating or alleviating the symptoms such as psychotic disorders or hyperactivity in a mammal such as a human.

The term “patient” refers to an animal including a mammal (e.g., a human).

The term “bioavailability” includes but is not limited to reference to the rate and extent to which an active ingredient or active moiety is absorbed from a drug product and becomes available at the site of action.

The term “Extended Release” includes but is not limited to reference to products which are formulated to make the drug available over an extended period after administration.

A formulation may include a hydrophilic matrix comprising a gelling agent, 11-[4-[2-(2-hydroxyethoxy)ethyl]-1-piperazinyl]dibenzo[b,f][1,4]thiazepine, or a pharmaceutically acceptable salt thereof, such as a hemifumarate salt, and one or more pharmaceutically acceptable excipients.

Examples of gelling agents that may be present in the embodiments of the invention include such substances as hydroxypropylcellulose, hydroxymethylcellulose, hydroxyethylcellulose, hydroxypropyl ethylcellulose, methylcellulose, carboxyethylcellulose, carboxymethyl hydroxyethylcellulose, carbomer, sodium carboxymethylcellulose, polyvinylpyrrolidone, and the like, or mixtures thereof. In certain embodiments, the gelling agent can comprise hypromellose.

The amount of gelling agent, in combination with the quetiapine and any excipients, may be selected such that the active ingredient is released from the formulation, in a controlled fashion, over a period of about 24 hours.

The gelling agent may be present in a range that is about 5 to 50% (by weight). The range may be about 5 to 40%. The range may be about 8 to 35%. The range may be about 10 to 35%. The range may be 10 to 30%. The range may be 15 to 30%. (Weight percentages, as used herein, are relative to the core tablet weight, excluding the weight of any coating, unless otherwise specified.)

Some embodiments of the invention may include hypromellose mixtures that include more than one grade of polymer. Polymers are commercially available under several trademarks, e.g. METHOCEL® E, F, J and K from the Dow Chemical Company, U.S.A. and METOLOSE® 60SH, 65SH and 90SH from Shin-Etsu, Ltd., Japan. The grades have differences in methoxy and hydroxypropoxy content as well as in viscosity and other characteristics. Different lots of hypromellose, even of the same grade may have differences in methoxy and hydroxypropoxy contents, viscosity and other characteristics.

The formulation may contain a buffer or pH modifier, for example if the active ingredient exhibits pH-dependent solubility , as is the case for quetiapine salts such as quetiapine fumarate.

The formulation will, in general, contain one or more excipients. Such excipients may include diluents such as lactose, microcrystalline cellulose, dextrose, mannitol, sucrose, sorbitol, gelatin, acacia, dicalcium phosphate, tricalcium phosphate, monocalcium phosphate, sodium phosphate, sodium carbonate and the like, preferably lactose and microcrystalline cellulose; lubricants such as stearic acid, zinc, calcium or magnesium stearate and the like, preferably magnesium stearate; binders such as sucrose, polyethylene glycol, povidone (polyvinylpyrrolidone), corn or maize starch, pregelatinized starch and the like; colorants such as ferric oxides, FD & C dyes, lakes and the like; flavoring agents; and pH modifiers that include suitable organic acids or alkali metal (e.g. lithium, sodium or potassium) salts thereof, such as benzoic acid, citric acid, tartaric acid, succinic acid, adipic acid and the like or the corresponding alkali metal salts thereof, preferably the alkali metal salts of such acids and in particular the sodium salt of citric acid (i.e. sodium citrate). As is well known, some excipients have multiple functions, such as being both a diluents and a binder.

In some embodiments of the invention, the formulation may be present in a solid dosage form such as a tablet, caplet or any other suitable form comprising 11-[4-[2-(2-hydroxyethoxy)ethyl]-1-piperazinyl]dibenzo[b,f][1,4]thiazepine hemifumarate (“quetiapine fumarate”), 6-18% by weight sodium citrate dihydrate, 30.0% by weight hydroxypropyl methylcellulose, wherein 15-29 of the 30.0% is a first hydroxypropyl methylcellulose constituent; the remainder of the 30.0% is a second hydroxypropyl methylcellulose constituent; and the first and second constituents correspond, respectively, to a first hydroxypropyl methylcellulose grade that has a apparent viscosity between 80 centipoise (“cp”) and 120 cp and a second hydroxypropyl methylcellulose that has a apparent viscosity between 3000 cp and 5600 cp. The tablet may comprise 11-12% by weight 11-[4-[2-(2-hydroxyethoxy)ethyl]-1-piperazinyl]dibenzo[b,f][1,4]thiazepine hemifumarate. The tablet may comprise 29.5-30.5% by weight 11-[4-[2-(2-hydroxyethoxy)ethyl]-1-piperazinyl]dibenzo[b,f][1,4]thiazepine hemifumarate. The tablet may comprise 37.9-38.9% by weight 11-[4-[2-(2-hydroxyethoxy)ethyl]-1-piperazinyl]dibenzo[b,f][1,4]thiazepine hemifumarate. In some embodiments, the tablet comprises 52.4-53.4% by weight 11-[4-[2-(2-hydroxyethoxy)ethyl]-1-piperazinyl]dibenzo[b,f][1,4]thiazepine hemifumarate Dosage forms may be manufactured in batches. A batch may include one or more constituents. A constituent may be commercially available and obtainable in lots. Dosage forms may be manufactured according to a “Batch Ratio Method,” in which variations in hydroxypropoxy content, which would be expected to cause variations in active ingredient release characteristics, may be offset by selection of an appropriate ratio (the “polymer ratio”) of high- and low-viscosity hypromellose. Effects on active ingredient release of variations in the properties of other constituents may be offset in the same way.

In some embodiments of the invention, the viscosities of the formulation are consistent with Ubbelohde viscosimeter viscosities of 2% by weight hydroxypropyl methylcellulose in 20° water, as determined using the method described in The United States Pharmacopoeia (USP30-NF25), United States Pharmacopoeia Convention, Inc. 2007, p. 2323, which is hereby incorporated by reference herein in its entirety.

In some embodiments of the invention, the formulation comprises sodium citrate dihydrate present in about 7.2-12.5% by weight. In some embodiments, the formulation comprises sodium citrate dihydrate present in 7.2% by weight. In some embodiments, the formulation comprises sodium citrate dihydrate present in 11.5% by weight. In some embodiments, the formulation comprises sodium citrate dihydrate present in 12.5% by weight.

In some embodiments of the invention, the formulation comprises lactose monohydrate present in up to about 30% by weight. In some embodiments, the formulation comprises lactose monohydrate present in 25.1% by weight. In some embodiments, the formulation comprises lactose monohydrate present in 13.0% by weight. In some embodiments, the formulation comprises lactose monohydrate present in 8.8% by weight. In some embodiments, the formulation comprises lactose monohydrate present in 1.8% by weight.

In some embodiments, the formulation comprises microcrystalline cellulose present in up to about 30% by weight. In some embodiments, the formulation comprises microcrystalline cellulose present in 25.1% by weight. In some embodiments, the formulation comprises microcrystalline cellulose present in 13.0% by weight. In some embodiments, the formulation comprises microcrystalline cellulose present in 8.8% by weight. In some embodiments, the formulation comprises microcrystalline cellulose present in 1.8% by weight.

In some embodiments, the tablet comprises an amount of magnesium stearate between about 1% and 3% by weight. In some embodiments, the tablet comprises magnesium stearate present in 1.0% by weight. In some embodiments, the tablet comprises magnesium stearate present in 1.5% by weight. In some embodiments, the tablet comprises magnesium stearate present in 2.0% by weight.

In some embodiments, the hydroxypropyl methylcellulose comprises 9.8 to 13.4% by weight of the hydroxypropyl methylcellulose, as measured by nuclear magnetic resonance (“NMR”), hydroxypropoxy. In some embodiments, the hydroxypropyl methylcellulose comprises 26.4 to 29.2% by weight of the hydroxypropyl methylcellulose, as measured by NMR, methoxy.

In some embodiments of the invention, the solid dosage form comprises 50 milligram (“mg”) quetiapine, for example in a 500 mg total core mass. In some embodiments, the solid dosage form comprises 150 mg quetiapine, for example, in a 575 mg total core mass. In some embodiments, the solid dosage comprises 200 mg quetiapine, for example in a 600 mg total core mass. In some embodiments, the solid dosage form comprises 400 mg quetiapine, for example in an 870 mg total core mass.

In some embodiments of the invention, the formulation is present in a solid dosage form comprising 50 mg quetiapine, the dosage form, after ingestion under steady state conditions by a human, resulting in a blood plasma concentration, in nanograms quetiapine per milliliter plasma, that is up to about: 67.6 at 1 hour after the ingestion; 124 at 4 hours after the ingestion; 105 at 8 hours after the ingestion; 74.3 at 12 hours after the ingestion; and 236 at 16 hours after the ingestion.

In some embodiments of the invention, the formulation is a solid dosage form comprising 200 mg quetiapine, the dosage form, after ingestion under steady state conditions by a human, resulting in a blood plasma concentration, in nanograms quetiapine per milliliter plasma, that is: up to about 251 at 1 hour after the ingestion; between about 32.2 and about 416 at 4 hours after the ingestion; up to about 496 at 8 hours after the ingestion; between about 4.6 and about 323 at 12 hours after the ingestion; and up to about 251 at 16 hours after the ingestion.

In some embodiments of the invention, the formulation is a solid dosage form comprising 400 mg quetiapine, the dosage form, after ingestion under steady state conditions by a human, resulting in a blood plasma concentration, in nanograms quetiapine per milliliter plasma, that is: between about 15.9 and about 391 at 1 hour after the ingestion; up to about 1052 at 4 hours after the ingestion; between about 63.1 and about 785 at 8 hours after the ingestion; between about 11.1 and about 613 at 12 hours after the ingestion; and up to about 448 at 16 hours after the ingestion.

In some embodiments of the invention, a dosage form comprises 30.0% by weight hydroxypropyl methylcellulose and 7.2% by weight sodium citrate dihydrate. In certain embodiments, 15-29 of the 30.0% is a first hydroxypropyl methylcellulose constituent; the remainder of the 30.0% is a second hydroxypropyl methylcellulose constituent; and the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp. In some embodiments, the viscosities of the dosage form are consistent with Ubbelohde viscosimeter viscosities of 2% by weight hydroxypropyl methylcellulose in 20° water, as determined using the method described in The United States Pharmacopoeia (USP30-NF25), United States Pharmacopoeia Convention, Inc. 2007, p. 2323. In some embodiments, the first and second constituents, respectively, have viscosities of 80-120 cp and 3000-5600 cp.

In some embodiments of the invention, a solid dosage form comprises 50 mg quetiapine, the dosage form, after ingestion under steady state conditions by a human, resulting in a time-dependent blood plasma quetiapine concentration, in nanograms quetiapine per milliliter plasma, having a maximum value, Cₘₐₓ, that is up to about 239 and corresponds to a time tₘₐₓ that is between 2 and 16 hours after the ingestion. In some embodiments, the concentration has a C₂₄ value, that is up to about 39.2 and corresponds to a time t₂₄, at 24 hours after the ingestion; and the ratio Cₘₐₓ:C₂₄ is up to about 35.2.

In some embodiments of the invention, a solid dosage form comprises 200 mg quetiapine, the dosage form, after ingestion under steady state conditions by a human, resulting in a time-dependent blood plasma quetiapine concentration, in nanograms quetiapine per milliliter plasma, having a maximum value, Cₘₐₓ, that is between about 3.9 and about 601 and corresponds to a time tₘₐₓ that is between 2 and 8 hours after the ingestion. In some embodiments, the concentration has a C₂₄ value that is up to about 156 and corresponds to a time t₂₄, at 24 hours after the ingestion; and the ratio Cₘₐₓ:C₂₄ is up to about 20.9.

In some embodiments of the invention, a solid dosage form comprises 400 mg quetiapine, the dosage form, after ingestion under steady state conditions by a human, resulting in a time-dependent blood plasma quetiapine concentration, in nanograms quetiapine per milliliter plasma, having a maximum value, Cₘₐₓ, that is between about 80 and about 1109 and corresponds to a time tₘₐₓ that is between 3 and 8 hours after the ingestion. In some embodiments, the concentration has a C₂₄ value that is up to about 265 and corresponds to a time t₂₄, at 24 hours after the ingestion; and the ratio Cₘₐₓ:C₂₄ is up to about 25.9.

In some embodiments of the invention, a solid dosage form comprises 50 mg quetiapine, the dosage form, after ingestion under steady state conditions by different humans, resulting in distinct time-dependent blood plasma quetiapine concentrations, which have a maximum value C_{ave,max} between about 5.1 and about 117 nanograms quetiapine per milliliter plasma, C_{ave,max} corresponding to a time that is between 2.5 and 3.5 hours after administration. In some embodiments, the distinct concentrations have an average value C_{ave,24} that is about 14.8 and corresponds to a time 24 hours after the ingestion; and the ratio C_{ave,max}:C_{ave,24} is about 4.1.

In some embodiments of the invention, a solid dosage form comprises 200 mg quetiapine, the dosage form, after ingestion under steady state conditions by different humans, resulting in distinct time-dependent blood plasma quetiapine concentrations, which have a maximum value C_{ave,max} that is up to about 550.4 nanograms quetiapine per milliliter plasma, C_{ave,max} corresponding to a time that is between 5.5 and 6.5 hours after administration. In some embodiments, the distinct concentrations have an average value C_{ave,24} that is about 64.9 and corresponds to a time 24 hours after the ingestion; and the ratio C_{ave,max}:C_{ave,24} is about 4.0.

In some embodiments of the invention, a solid dosage form comprises 400 mg quetiapine, the dosage form, after ingestion under steady state conditions by different humans, resulting in distinct time-dependent blood plasma quetiapine concentrations, which have a maximum value C_{ave,max} that is up to about 1062 nanograms quetiapine per milliliter plasma, C_{ave,max} corresponding to a time that is between 2.5 and 4.5 hours after administration. In some embodiments, the distinct concentrations have an average value C_{ave,24} that is about 114 and corresponds to a time 24 hours after the ingestion; and the ratio C_{ave,max}:C_{ave,24} is about 4.6.

In some embodiments of the invention, a solid dosage form comprises 50 mg quetiapine, the dosage form, afteringestion under steady state conditions by different humans, resulting in distinct time-dependent blood plasma quetiapine concentrations, which have a cumulative area-under-the-curve, AUC_{cum}, that is: up to 46 at 1 hour after ingestion; between 8 and 352 at 4 hours after ingestion; between 34 and 789 at 8 hours after ingestion; between 83 and 1092 at 12 hours after ingestion; between 111 and 1396 at 16 hours after ingestion; and up to 1935 at 24 hours after ingestion; wherein AUC_{cum} has units of (nanogram quetiapine)×hour/milliliter.

In some embodiments of the invention, a solid dosage form comprises 200 mg quetiapine, the dosage form, afteringestion under steady state conditions by different humans, resulting in distinct time-dependent blood plasma quetiapine concentrations, which have a cumulative area-under-the-curve, AUC_{cum}, that is:up to 177 at 1 hour after ingestion; between 35 and 1318 at 4 hours after ingestion; between 188 and 3115 at 8 hours after ingestion; between 251 and 4650 at 12 hours after ingestion; between 362 and 5666 at 16 hours after ingestion; and between 441 and 6899 at 24 hours after ingestion; wherein AUC_{cum} has units of (nanogram quetiapine)×hour/milliliter.

In some embodiments of the invention, a solid dosage form comprises 400 mg quetiapine, the dosage form, after ingestion under steady state conditions by different humans, resulting in distinct time-dependent blood plasma quetiapine concentrations, which have a cumulative area-under-the-curve, AUC_{cum}, that is between: 3 and 320 at 1 hour after ingestion; 143 and 2677 at 4 hours after ingestion; 575 and 6158 at 8 hours after ingestion; 916 and 8722 at 12 hours after ingestion; 1037 and 10685 at 16 hours after ingestion; 1031 and 13033; and 1031 and 13033 at 24 hours after ingestionn; wherein AUC_{cum} has units of (nanogram quetiapine)×hour/milliliter.

In some embodiments of the invention, a formulation comprises quetiapine fumarate and 30.0% hydroxypropyl methylcellulose, wherein 15-29 of the 30.0% is a first hydroxypropyl methylcellulose constituent, such that the formulation satisfies a predetermined dissolution criterion; the remainder of the 30.0% is a second hydroxypropyl methylcellulose constituent; the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp.

In some embodiments, the formulation comprises 11-12% by weight quetiapine fumarate. In some embodiments, the formulation comprises 29.5-30.5% by weight quetiapine fumarate. In some embodiments, the formulation comprises 37.9-38.9% by weight quetiapine fumarate. In some embodiments, the formulation comprises 52.4-53.4% by weight quetiapine fumarate.

In some embodiments, when dissolution of the formulation takes place in a basket apparatus having a rotation speed of 200 revolutions per minute and containing 900 milliliter 0.05 molar sodium citrate and 0.09 normal sodium hydroxide, to which 100 milliliter 0.05 molar sodium phosphate and 0.46 normal sodium hydroxide are added after 5 hours: no more than 20% of the quetiapine is dissolved during the first one-hour period of the dissolution. In some embodiments, 47-69% of the quetiapine is dissolved during the first 6-hour period of the dissolution. In some embodiments, 65-95% of the quetiapine is dissolved during the first 12-hour period of the dissolution. In some embodiments, at least 85% of the quetiapine is dissolved during the first 20-hour period of the dissolution.

In some embodiments of the invention, a formulation comprises quetiapine fumarate and 30.0% hydroxypropyl methylcellulose, wherein 15-29 of the 30.0% is a first hydroxypropyl methylcellulose constituent, such that the formulation optimally exhibits at least one dissolution target; the remainder of the 30.0% is a second hydroxypropyl methylcellulose constituent; the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp.

In some embodiments, the formulation comprises 11-12% by weight quetiapine fumarate. In some embodiments, the formulation comprises 29.5-30.5% by weight quetiapine fumarate. In some embodiments, the formulation comprises 37.9-38.9% by weight quetiapine fumarate. In some embodiments, the formulation comprises 52.4-53.4% by weight quetiapine fumarate.

In some embodiments, a first target is, when dissolution takes place in a basket apparatus having a rotation speed of 200 revolutions per minute and containing 900 milliliter 0.05 molar sodium citrate and 0.09 normal sodium hydroxide, to which 100 milliliter 0.05 molar sodium phosphate and 0.46 normal sodium hydroxide are added after 5 hours: 58% of the quetiapine is dissolved in the first six-hour period of the dissolution. In some embodiments, a second target is: 80% of the quetiapine is dissolved in the first 12-hour period of the dissolution.

In some embodiments of the invention, a solid dosage form comprises a dose of quetiapine, the dosage form, after ingestion under steady state conditions by different humans, resulting in time-dependent blood plasma quetiapine concentrations that, the average of which have a dose-scaled concentration, C/dose, that is between: about 0.433 and about 0.678 at 1 hour after administration; about 1.01 and about 1.35 at 4 hours after administration; about 0.930 and about 1.35 at 8 hours after administration; about 0.590 and about 1.07 at 12 hours after administration; and about 0.204 and about 1.22 at 16 hours after administration; wherein the dose is between 49.5 mg and 249.5 mg and C is expressed in nanogram quetiapine per milliliter plasma.

In some embodiments of the invention, a solid dosage form comprises a dose of quetiapine, the dosage form, after ingestion under steady state conditions by different humans, resulting in time-dependent blood plasma quetiapine concentrations, the average of which have a dose-scaled concentration, C/dose, that is between: about 0.433 and about 0.678 at 1 hour after administration; about 1.01 and about 1.35 at 4 hours after administration; about 0.930 and about 1.35 at 8 hours after administration; about 0.590 and about 1.07 at 12 hours after administration; and about 0.204 and about 1.22 at 16 hours after administration; wherein the dose is greater than 350 mg and C is expressed in nanogram quetiapine per milliliter plasma.

In some embodiments of the invention, a solid dosage form comprises an amount of quetiapine and 30.0% hydroxypropyl methylcellulose, wherein 15-29 of the 30.0% is a first hydroxypropyl methylcellulose constituent, such that the formulation optimally exhibits the time-dependent ratio C dose; the remainder of the 30.0% is a second hydroxypropyl methylcellulose constituent; the first and second constituents correspond, respectively, to a first hydroxypropyl methylcellulose grade that has an apparent viscosity between 80 cp and 120 cp and a second hydroxypropyl methylcellulose that has an apparent viscosity between 3000 cp and 5600 cp; and C dose is within a range defined by

$$base + \frac{exp ( - {K}_{a} \times t ) - exp ( - {K}_{e} \times t )}{{K}_{e} / {K}_{a} - 1.5} ,$$

in which: C is the average quetiapine blood plasma concentration, in nanogram quetiapine per milliliter plasma, at time t after administration of the quetiapine to a human; base is between, inclusively, 0.1227 and 0.2428; Kₑ is between, inclusively, 0.2344 and 0.2678; Kₐ is between, inclusively, 0.1396 and 0.1592; and the dose is between 49.5 mg and 249.5 mg.

In some embodiments, a solid dosage form comprises an amount of quetiapine and 30.0% hydroxypropyl methylcellulose, wherein 15-29 of the 30.0% is a first hydroxypropyl methylcellulose constituent, such that the formulation optimally exhibits a time-dependent ratio C:dose; the remainder of the 30.0% is a second hydroxypropyl methylcellulose constituent; the first and second constituents correspond, respectively, to a first hydroxypropyl methylcellulose grade that has a apparent viscosity between 80 cp and 120 cp and a second hydroxypropyl methylcellulose that has a apparent viscosity between 3000 cp and 5600 cp; and C:dose is within a range defined by

$$base + \frac{exp ( - {K}_{a} \times t ) - exp ( - {K}_{e} \times t )}{{K}_{e} / {K}_{a} - 1.5} ,$$

in which: C is the average quetiapine blood plasma concentration, in nanogram quetiapine per milliliter plasma, at time t after administration of the quetiapine to a human; base is between, inclusively, 0.1227 and 0.2428; Kₑ is between, inclusively, 0.2344 and 0.2678; Kₐ is between, inclusively, 0.1396 and 0.1592; and the dose is greater than 350 mg.

The invention may include a method for manufacturing a solid dose form having a composition that includes an active ingredient and first and second constituents. The active ingredient may be quetiapine. In some embodiments of the invention, the method may comprise inputting into a multivariate model first data corresponding to a first constituent; inputting into the model second data corresponding to a second constituent; using the model, identifying a ratio between a first constituent amount and a second constituent amount such that the dosage form satisfies a dissolution criterion when the composition includes the first and second constituents in proportion to the ratio. This method may be used, for example, to find a constituent ratio to obtain a desired dissolution profile in the face of variations in constituent properties, such as lot-to-lot or source-to-source variations, that may occur during the dosage form manufacture, such as commercial scale manufacture over an extended period of time, such as when identifcal constituent lots may not be readily available.

In some embodiments, the first and second constituents comprise, respectively, first and second hydroxypropyl methylcellulose lots. In some embodiments, the first and second lots have first and second viscosities, respectively, and the first viscosity is different from the second viscosity. In some embodiments, the first viscosity is in the range 80-120 cp, and the second viscosity is in the range 3000-5600 cp.

In some embodiments, the first and second data comprise measured viscosities corresponding to the first and second lots, respectively. In some embodiments, the first and second data comprise hydroxypropoxy contents of the first and second lots, respectively. In some embodiments, at least one of the hydroxypropoxy contents is measured using nuclear magnetic resonance. In some embodiments, at least one of the methoxy contents is measured using nuclear magnetic resonance.

In some embodiments, the first and second data comprise molecular weights corresponding to the first and second lots, respectively.

In some embodiments, the first and second data comprise methoxy contents of the first and second lots, respectively.

In some embodiments, the first and second data comprise particle size information corresponding to the first and second lots, respectively. Particle size information may be characterized as %-through-100-mesh (an index that may be taken from the suppliers certificate of analysis; smaller sieve “mesh” sizes of 3½ to 400 are designated by the number of openings per linear inch in the sieve. Thus, a 100 mesh sieve has 100 openings per inch. For example, a 100 mesh sieve may have holes that are 149×149 microns. % through a 100 mesh sieve is therefore the percentage by weight of particles that are less than 149 microns in diameter.). Particle size may also be characterized as average particle diameter (D50) and/or particle size span, both of which may be determined using a laser diffraction technique.

In some embodiments, the first and second data comprise molecular number information corresponding to the first and second lots, respectively.

In some embodiments, the method comprises inputting into the model a quetiapine salt content corresponding to the composition.

In some embodiments, the method comprises inputting into the model an excipient content corresponding to the composition.

In some embodiments, the method comprises inputting the dosage form weight into the model.

In some embodiments, the method comprises inputting into the model a quetiapine amount corresponding to the composition; wherein the first and second data comprise, with respect to the first and second lots, respectively: hydroxypropoxy contents; and molecular weight information. In some embodiments, the hydroxypropoxy contents are characterized as weight percentages of a total hydroxypropyl methylcellulose weight.

In some embodiments, the ratio of the first to the second component has: a minimum value of 15% composition weight:15% composition weight; and a maximum value of 29% composition weight:1% composition weight.

In some embodiments, the dissolution criterion is satisfied when the formulation in a solid dosage form, when subjected to predetermined conditions for a time, dissolves to an extent that is within a predetermined range. In some embodiments, the dissolution criterion is satisfied when the extent is optimal within the range.

In some embodiments, when the ratio is a first ratio, using the model includes predicting dissolution for a second ratio; and the dissolution extent is optimal when the extent is closer to the center of the range than is the dissolution corresponding to the second ratio.

The invention may include a method for manufacturing a dosage form by establishing for first and second properties of first and second constituents, respectively, a correlation between a ratio and dissolution profile information; wherein the ratio is between a first constituent amount and a second constituent amount such that the dosage form satisfies a dissolution criterion when the composition includes the first and second constituents in proportion to the ratio.

In some embodiments, the first property promotes dissolution; and the second property retards dissolution. In some embodiments, the first property corresponds to hydroxypropoxy content.

In some embodiments, the second property corresponds to viscosity, molecular weight, or molecular number.

In some embodiments, the first property corresponds to hydroxypropoxy content and the second property corresponds to viscosity.

In some embodiments, the dissolution profile information includes a first value corresponding to a time and a second value corresponding dissolution extent at the time.

In some embodiments, the correlation may be embodied in a multivariate model.

The method may include measuring the hydroxypropoxy and methoxy of a plurality of batches of hydroxypropyl methylcellulose. In some embodiments the measuring is implemented using nuclear magnetic resonance (NMR). A first grade of the hypromellose has a first viscosity and a second grade may have a second viscosity. The method may include inputting into a multivariate model the tablet strength and the hydroxypropoxy content and molecular weight of each of the first grade and the second grade. The method may also include inputting into the model a series of ratios between an amount of the first grade and an amount of the second grade. The method may also include identifying, using the model, an optimum ratio that corresponds to a predicted dissolution profile that has a smaller deviation from a target profile than the deviation obtained using the other ratios. Alternatively, the method may include identifying, using the model, at least one ratio that produces a formulation that satisfies a desired dissolution profile.

In some embodiments, the model may be an artificial neural network (“ANN”) model.

In some embodiments, the correlation may be embodied in a look-up table.

Exemplary formulations for tablet strengths 50 mg, 150 mg, 200 mg, 300 mg and 400 mg are shown in Tables 1-5, respectively:

TABLE 1
Tablet strength: 50 mg

| Ingredients | Mass (mg) | % by weight |
|---|---|---|
| Quetiapine fumarate¹ | 57.56 | 11.5 |
| (quetiapine) | (50.00) | (10.0) |
| Lactose monohydrate | 125.72 | 25.1 |
| Microcrystalline cellulose | 125.72 | 25.1 |
| Sodium citrate dihydrate | 36.00 | 7.2 |
| hypromellose 2208 100 cp | 120.00 | 24.0 |
| hypromellose 2208 4000 cp | 30.00 | 6.0 |
| Magnesium stearate | 5.00 | 1.0 |
| Purified water | qs | — |
| Total Tablet Weight | 500.00 | 100.0 |

¹Quetiapine fumarate contains 86.86% by weight quetiapine

TABLE 2
Tablet strength: 150 mg

| Ingredients | Mass (mg) | % by weight |
|---|---|---|
| Quetiapine fumarate¹ | 172.69 | 30.0 |
| (quetiapine) | (150.00) | (26.1) |
| Lactose monohydrate | 74.65 | 13.0 |
| Microcrystalline cellulose | 74.65 | 13.0 |
| Sodium citrate dihydrate | 71.88 | 12.5 |
| hypromellose 2208 100 cp | 120.75 | 21.0 |
| hypromellose 2208 4000 cp | 51.75 | 9.0 |
| Magnesium stearate | 8.63 | 1.5 |
| Purified water | qs | — |
| Core Tablet Weight | 575.00 | 100.0 |

¹Quetiapine fumarate contains 86.86% by weight quetiapine

TABLE 3
Tablet strength: 200 mg

| Ingredients | Mass (mg) | % by weight |
|---|---|---|
| Quetiapine fumarate¹ | 230.26 | 38.4 |
| (quetiapine) | (200.00) | (33.3) |
| Lactose monohydrate | 52.87 | 8.8 |
| Microcrystalline cellulose | 52.87 | 8.8 |
| Sodium citrate dihydrate | 75.00 | 12.5 |
| hypromellose 2208 100 cp | 138.00 | 23.0 |
| hypromellose 2208 4000 cp | 42.00 | 7.0 |
| Magnesium stearate | 9.00 | 1.5 |
| Purified water | qs | — |
| Core Tablet Weight | 600.00 | 100.0 |

¹Quetiapine fumarate contains 86.86% by weight quetiapine

TABLE 4
Tablet strength: 300 mg

| Ingredients | Mass (mg) | % by weight |
|---|---|---|
| Quetiapine fumarate¹ | 345.38 | 43.2 |
| (quetiapine) | (300.00) | (37.5) |
| Lactose monohydrate | 49.31 | 6.2 |
| Microcrystalline cellulose | 49.31 | 6.2 |
| Sodium citrate dihydrate | 100.00 | 12.5 |
| hypromellose 2208 100 cp | 200.00 | 26.0 |
| hypromellose 2208 4000 cp | 40.00 | 4.0 |
| Magnesium stearate | 16.00 | 2.0 |
| Purified water | qs | — |
| Core Tablet Weight | 800.00 | 100.0 |

¹Quetiapine fumarate contains 86.86% by weight quetiapine

TABLE 5
Tablet strength: 400 mg

| Ingredients | Mass (mg) | % by weight |
|---|---|---|
| Quetiapine fumarate¹ | 460.50 | 52.9 |
| (quetiapine) | (400.00) | (46.0) |
| Lactose monohydrate | 15.50 | 1.8 |
| Microcrystalline cellulose | 15.60 | 1.8 |
| Sodium citrate dihydrate | 100.00 | 11.5 |
| hypromellose 2208 100 cp | 234.90 | 27.0 |
| hypromellose 2208 4000 cp | 26.10 | 3.0 |
| Magnesium stearate | 17.40 | 2.0 |
| Purified water | qs | — |
| Core Tablet Weight | 870.00 | 100.0 |

¹Quetiapine fumarate contains 86.86% by weight quetiapine

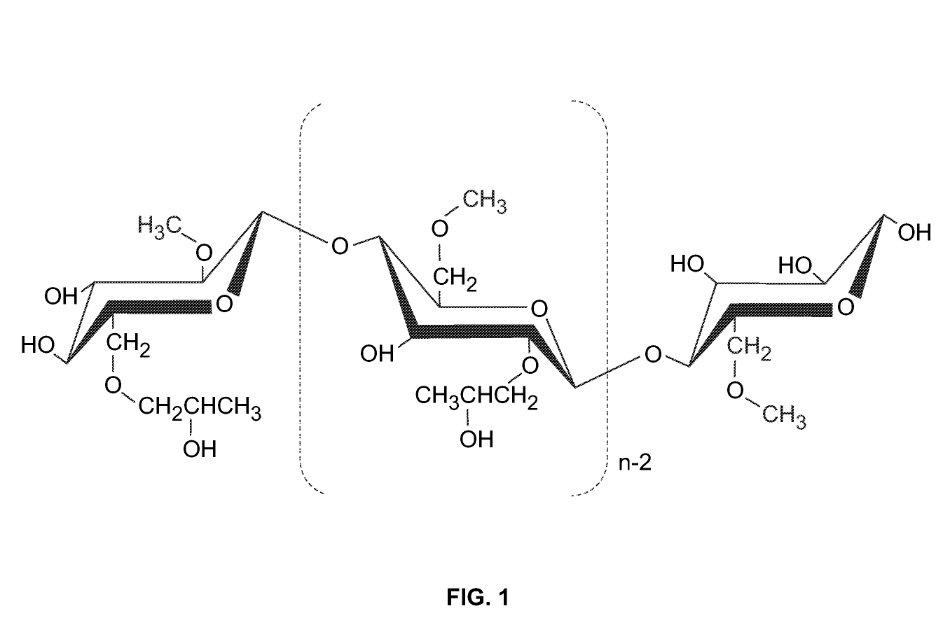

FIG. 1 shows units of substituted anhydroglucose that make up hypromellose and are involved in dissolution processes that will be discussed in more detail below in connection with certain exemplary embodiments.

The formulations may be embodied in extended release 50, 150, 200, 300 and 400 mg tablets that may be manufactured using one or more of the following devices and processes: standard high shear wet granulation, fluid bed dryer, milling, blending, compression, aqueous film coating processes, and any other suitable processes that are the same or similar to other manufacturing processes used throughout the pharmaceutical industry.

Raw materials may be transferred into the high-shear granulator and may be mixed for 10 minutes. All excipients (with the exception of magnesium stearate) may be added to the high shear granulator. A dry mix time of 10 minutes may be used.

During the wet granulation stage water may be added to the dry mix to complete the granulation. There may be a range in both the amount of water added to the granulation and in the rate of water addition to provide an acceptable product.

Wet-milled material may be dried in a fluid bed dryer. For each batch moisture a target of <3% loss on drying (LOD) may be achieved.

An impact mill may be used for size reduction of the granulation to provide adequate flow and compression characteristics.

A lubricant blending time of 3 minutes may be used.

Illustrative tablet processing parameters for two different commercial plants are shown in Table 6.

TABLE 6

| | 50 mg | | 200 mg | | 300 mg | | 400 mg | |
|---|---|---|---|---|---|---|---|---|
| | Plant | | | | | | | |
| | 1 | 2 | 1 | 2 | 1 | 2 | 1 | 2 |
| Dry mix time | 10 | 10 | 10 | 10 | 10 | 10 | 10 | 10 |
| (min) | | | | | | | | |
| Amount of | 151.2 | 187.6 | 163.0 | 240.0 | 242.0 | 300.3 | 263.1 | 320.3 |
| water | | | | | | | | |
| (mg/tablet) | | | | | | | | |
| Water addition | 5 | 11 | 9 | 12 | 10 | 11 | 10 | 10 |
| rate | | | | | | | | |
| (kg/minute) | | | | | | | | |
| Extra | 0.5 | 0.5 | 0.5 | 0.5 | 0.5 | 0.5 | 0.5 | 0.5 |
| granulation | | | | | | | | |
| time (minutes) | | | | | | | | |
| Loss on drying | ≦3.0 | ≦3.0 | ≦3.0 | ≦3.0 | ≦3.0 | ≦3.0 | ≦3.0 | ≦3.0 |
| (% w/w) | | | | | | | | |
| Mill screen | 3.0 | 1.3 | 2.5 | 1.7 | 2.5 | 2.4 | 3.0 | 2.8 |
| (mm) | | | | | | | | |
| Tablet weight | 500 | 500 | 600 | 600 | 800 | 800 | 870 | 870 |
| (mg) | | | | | | | | |
| Hardness (kp) | ≧20 | ≧20 | ≧20 | ≧20 | ≧20 | ≧20 | ≧20 | ≧20 |
| Friability | ≦1% | ≦1% | ≦1% | ≦1% | ≦1% | ≦1% | ≦1% | ≦1% |

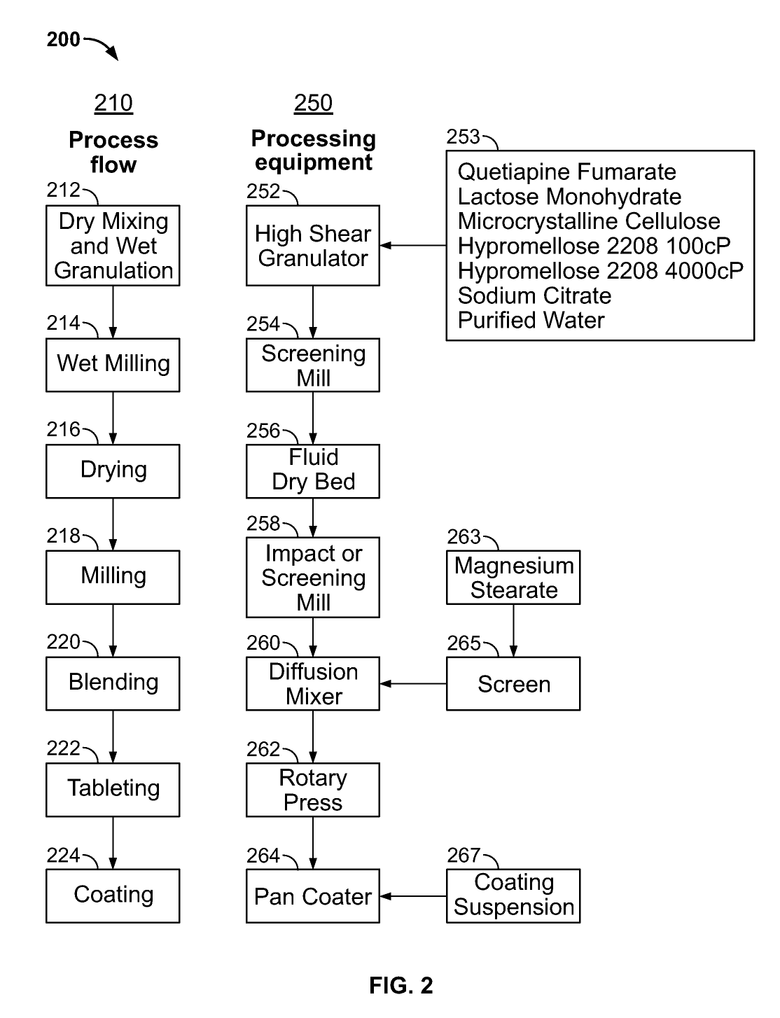

FIG. 2 shows an illustrative flow diagram for the manufacture of quetiapine fumarate tablets. Manufacturing process 200 may include process flow 210 and processing equipment 250. Process flow may include dry mixing and wet granulation 212 using high shear granulator 252, wet milling 214 using screening mill 254, drying 216 using fluid bed dryer 256, milling 218 using impact or screening mill 258, blending 220 using diffusion mixer 260, tableting 222 using rotary press 262 and coating 224 using pan coater 264. Flow 210 and equipment 250 are merely exemplary and other suitable flow steps as well as processing equipment may be used.

In connection with step 253 an exemplary list of constituents to be dry mixed and wet granulated by high shear granulator 252 is shown. Magnesium stearate 263 may be added through screen 265 during blending 220. Coating suspension 267 may be included in coating process 224.

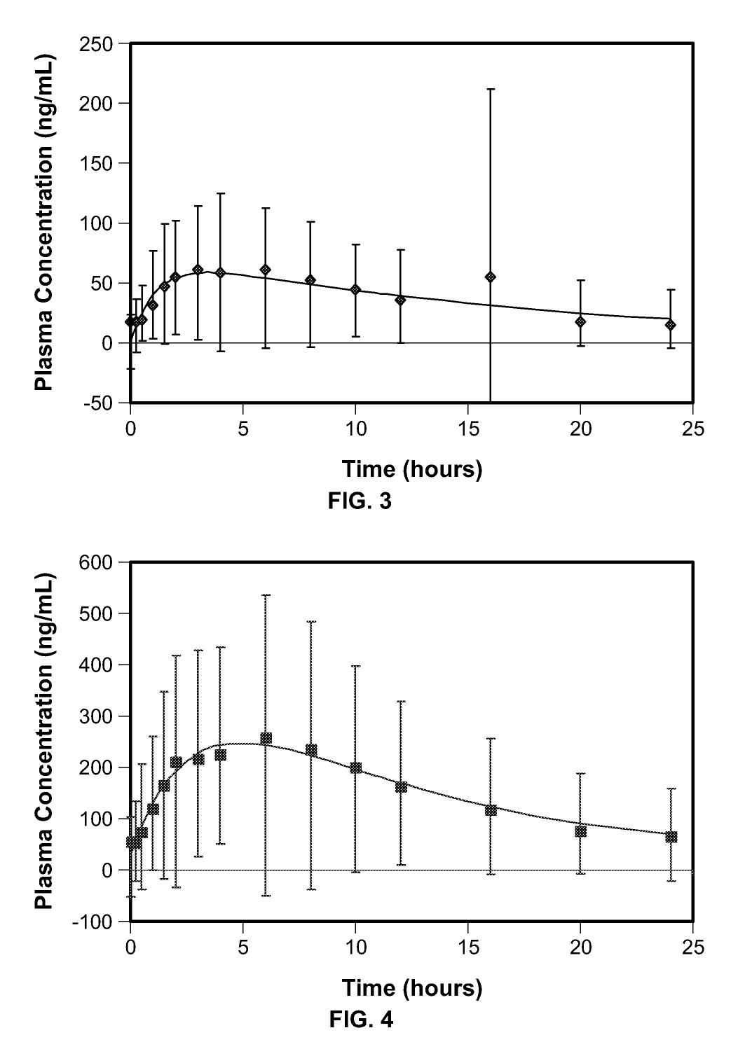

The following protocol was used to determine the blood plasma concentrations of active ingredient in patients. FIGS. 3-6 show plasma concentration—time plots (mean and range).

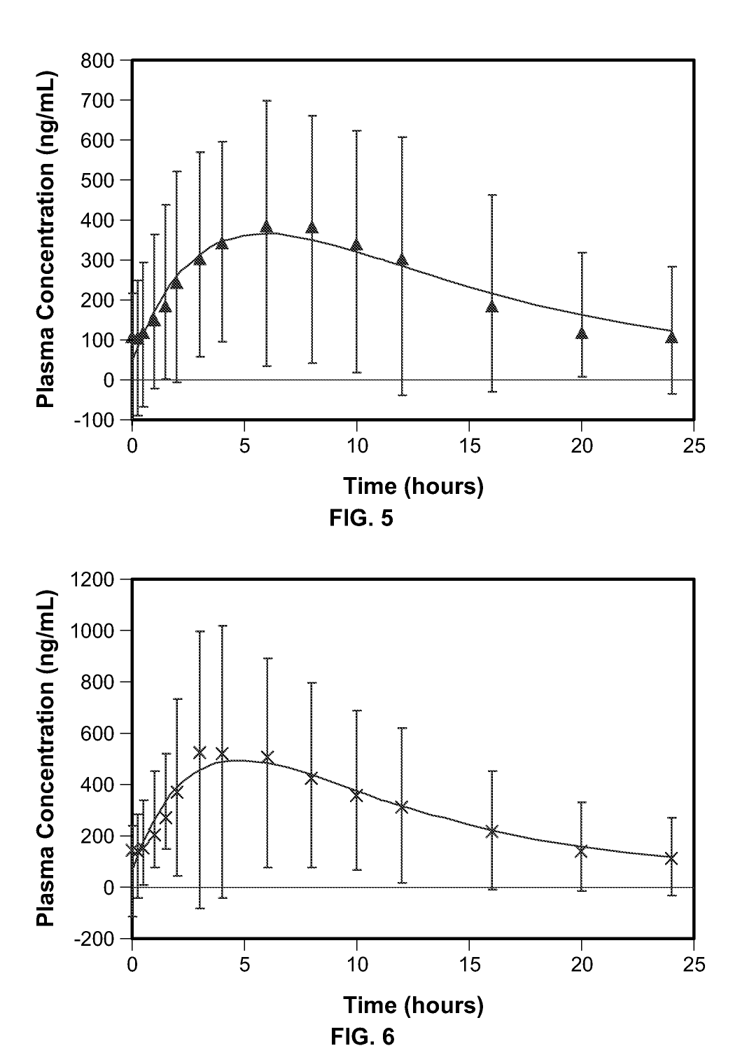

A multicenter, open-label, multiple-dose study was performed to evaluate the steady-state pharmacokinetics of commercial-scale tablets comprising study formulations (“SF”) having the following quetiapine strengths: 50 mg, 200 mg, 300 mg and 400 mg. The study formulations have compositions that are set forth in Tables 1-5. After a 2-day washout period, patients received oral doses of the study formulations and immediate-release (“IR”) medicament that is now available under the trademark “Seroquel” (available from AstraZeneca Pharmaceuticals, Wilmington, Del.) once daily as follows: 50 mg SF on Days 1 to 4, 200 mg SF on Days 5 to 7, 300 mg SF on Days 8 to 11, 400 mg SF on Days 12 to 14 and 300 mg IR on Days 15 to 17. On Days 4 and 11, patients consumed a standardized high-fat breakfast within 10 minutes of their scheduled dose. Data from Day 3 (50 mg; FIG. 3), Day 7 (200 mg; FIG. 4), Day 10 (300 mg; FIG. 5) and Day 14 (400 mg; FIG. 6) were used and it was assumed that steady-state had been achieved for each dose level. In each plot (FIGS. 3-6), error bars correspond to a 95% prediction interval.

Data from the study are set forth in Tables 6A and 6B. In Table 6A, Cₜ is concentration, in nanograms per milliliter plasma, at a time, t, which is expressed in hours after ingestion of the tablet. AUC_{cum}_{ₜ} is cumulative area-under-the-concentration-curve, in (nanogram quetiapine)×hour/milliliter, at a time t, which is expressed in hours after ingestion of the tablet. Quantities shown in Table 6A that are derived from Cₜ and AUC_{cum}_{ₜ} are explained above. Table 6A.

| | 50 mg | | | 200 mg | | | 300 mg | | | 400 mg | | |
|---|---|---|---|---|---|---|---|---|---|---|---|---|
| | L¹ | M² | U³ | L | M | U | L | M | U | L | M | U |
| C₁ | −5.7 | 30.9 | 67.6 | −11.4 | 120 | 251 | −43.8 | 149 | 341 | 15.9 | 203 | 391 |
| C₄ | −8.0 | 57.9 | 124 | 32.2 | 224 | 416 | 92.4 | 342 | 592 | −6.5 | 523 | 1052 |
| C₈ | −0.1 | 52.2 | 105 | −24.9 | 236 | 496 | 71.4 | 381 | 692 | 63.1 | 424 | 785 |
| C₁₂ | −2.6 | 35.9 | 74.3 | 4.6 | 164 | 323 | −22.3 | 301 | 624 | 11.1 | 312 | 613 |
| C₁₆ | −126 | 55.0 | 236 | −15.6 | 118 | 251 | −62.2 | 185 | 431 | −10.9 | 218 | 448 |
| Cₘₐₓ | −51.5 | 93.9 | 239 | 3.9 | 302 | 601 | 119 | 465 | 810 | 80 | 594 | 1109 |
| tₘₐₓ⁴ | 2 | 6 | 16 | 2 | 6 | 8 | 2 | 6 | 10 | 3 | 3, 6 | 8 |
| C₂₄ | −9.5 | 14.8 | 39.2 | −25.7 | 64.9 | 156 | −54.6 | 105 | 266 | −38.1 | 114 | 265 |
| Cₘₐₓ:C₂₄ | −10.9 | 12.1 | 35.2 | −5.8 | 7.5 | 20.9 | −5.7 | 8.0 | 21.6 | −8.1 | 8.9 | 25.9 |
| C_{ave,max}⁵ | 5.1 | 60.9 | 117 | −35.3 | 258 | 550.4 | 54.3 | 385 | 717 | −16.8 | 523 | 1062 |
| t_{ave,max} | | 3 | | | 6 | | | 6 | | | 3 | |
| C_{ave,24} | | 14.8 | | | 64.9 | | | 105 | | | 114 | |
| C_{ave,max}:C_{ave,24} | | 4.1 | | | 4.0 | | | 3.7 | | | 4.6 | |
| AUC_{cum,1} | −4 | 21 | 46 | −21 | 78 | 177 | −57 | 120 | 297 | 3 | 162 | 320 |
| AUC_{cum,4} | 8 | 180 | 352 | 35 | 677 | 1318 | 38 | 904 | 1771 | 143 | 1410 | 2677 |
| AUC_{cum,8} | 34 | 411 | 789 | 188 | 1652 | 3115 | 575 | 2399 | 4223 | 575 | 3367 | 6158 |
| AUC_{cum,12} | 83 | 587 | 1092 | 251 | 2450 | 4650 | 958 | 3757 | 6555 | 916 | 4819 | 8722 |
| AUC_{cum,16} | 111 | 753 | 1396 | 362 | 3014 | 5666 | 1062 | 4699 | 8336 | 1037 | 5861 | 10658 |
| AUC_{cum,24} | −43 | 946 | 1935 | 441 | 3670 | 6899 | 917 | 5734 | 10551 | 1031 | 7032 | 13033 |

¹Lower Confidence Limit for individual subject data (2-sided, p = 0.05, n = 12)
²Mean for individual subject data (n = 12)
³Upper Confidence Limit for individual subject data (2-sided, p = 0.05, n = 12)
⁴Minimum, most frequent and maximum observed values
⁵The maximum value of Cₐᵥₑ, the average plasma concentration for all subjects at a single time-point

In Table 6B, C/doseₜ is a strength-independent ratio of concentration, in nanograms quetiapine per milliliter plasma, to tablet strength, in mg quetiapine, at a time, t, which is expressed in hours after ingestion of the tablet.

TABLE 6B

| | L¹ | M² | U³ |
|---|---|---|---|
| C/dose₁ | 0.433 | 0.556 | 0.678 |
| C/dose₄ | 1.01 | 1.18 | 1.35 |
| C/dose₈ | 0.930 | 1.14 | 1.35 |
| C/dose₁₂ | 0.590 | 0.830 | 1.07 |
| C/dose₁₆ | 0.204 | 0.713 | 1.22 |

¹Lower Confidence Limit for C/dose calculated from Cₐᵥₑ for each strength for each timepoint (2-sided, p = 0.05, n = 4)
²Grand Mean of C/dose calculated from Cave for each strength for each timepoint (n = 4)
³Upper Confidence Limit for C/dose calculated on same basis as LCL

Each plot (FIGS. 3-6) also shows a best fit curve based on a pharmacokinetic (“PK”) model using first-order drug absorption and elimination rate constants Kₑ and Kₐ, respectively, with the equation

$$Y = base + ( \frac{1000 ( exp ( - {K}_{a} \times t ) - exp ( - {K}_{e} \times t ) )}{{K}_{e} / {K}_{a} - 1.5} ) .$$

### (See, e.g., “The Time Course of Drug Action,” Neubig, R. R., in Principles of Drug Action, Pratt, W, B., Taylor, P., (Eds), 3^{rd} Edition, Churchill Livingstone, Inc. 1990.)

The PK model parameters, best fit values and standard errors (“SE”), along with 95% confidence interval, for active ingredient amounts 50 mg, 200 mg, 300 mg and 400 mg, respectively, are set forth in Tables 7-10, which correspond to the data shown in FIGS. 3-6, respectively.

TABLE 7

| PK Model | | |
|---|---|---|
| Parameter | Estimate (best fit value) | SE (95% CI) |
| Base | 0.3733 | 3.293 (−6.077 to 6.832) |
| Ke | 0.8421 | 0.08356 (0.6783 to 1.006) |
| Ka | 0.05765 | 0.005473 (0.04693 to 0.06838) |

TABLE 8

| PK Model | | |
|---|---|---|
| Parameter | Estimate (best fit value) | SE (95% CI) |
| Base | 25.86 | 12.54 (1.285 to 50.44) |
| Ke | 0.3541 | 0.03004 (0.2953 to 0.4130) |
| Ka | 0.1033 | 0.008447 (0.08678 to 0.1199) |

TABLE 9

| PK Model | Estimate (best fit value) | SE (95% CI) |
|---|---|---|
| Base | 42.15 | 18.05 (6.874 to 77.52) |
| Ke | 0.2592 | 0.01879 (0.2224 to 0.2960) |
| Ka | 0.1033 | 0.007459 (0.08872 to 0.1180) |

TABLE 10

| PK Model | Estimate (best fit value) | SE (95% CI) |
|---|---|---|
| Base | 62.96 | 22.87 (18.13 to 107.8) |
| Ke | 0.2959 | 0.01975 (0.2571 to 0.3346) |
| Ka | 0.1390 | 0.008857 (0.1217 to 0.1564) |

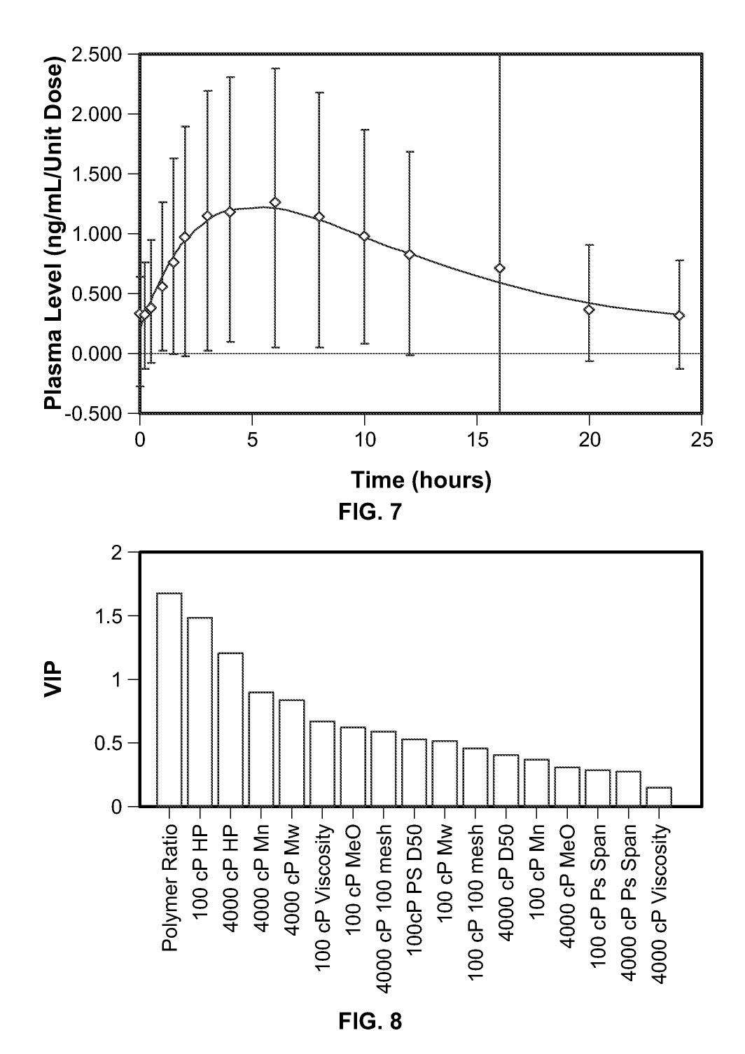

The PK model parameters, best fit values and standard errors (95% confidence interval) for the dose-normalized curve are set forth in Table 11, which corresponds to the data shown in FIG. 7. Error bars correspond to the 95% confidence interval.

TABLE 11

| PK Model | | |
|---|---|---|
| Parameter | Estimate (best fit value) | SE (95% CI) |
| Base | 0.1828 | 0.03065 (0.1227 to 0.2428) |
| Ke | 0.2511 | 0.008518 (0.2344 to 0.2678) |
| Ka | 0.1494 | 0.005004 (0.1396 to 0.1592) |

Hypromellose rapidly hydrates following ingestion to form a continuous gel layer. The gel layer acts initially to prevent wetting and consequent disintegration of the tablet core, which would lead to rapid and complete release of drug, then subsequently to mediate drug release via a complex mechanism that involves inward extension of the hydrated gel layer, swelling, diffusion of drug through the gel to the surrounding medium, and erosion that results in the release of active ingredient and hypromellose from the outer surface. See “Using Dow Excipients for Controlled Release of Drugs in Hydrophilic Matrix Systems” Technical Guide published by the Dow Chemical Company, September, 2006, which is hereby incorporated by reference herein in its entirety.

Hypromellose is a cellulose ether derived by chemical modification of cellulose, a naturally occurring carbohydrate that contains a repeating structure of anhydroglucose units. Cellulose itself is an insoluble fibrous polymer; however, each anhydroglucose unit contains 5 reactive hydroxyl groups, two of which are utilized in chain propagation, which leaves three sites for chemical substitution. For pharmaceutical applications, the most commonly used substituents are methyl, ethyl, and hydroxypropyl. Ethyl celluloses are insoluble in water but are soluble in certain organic solvents and have utility, either alone or in combination with other excipients, as tablet coatings or in the manufacture of hydrophobic matrix tablets. Methyl celluloses are generally soluble in water, while hydroxypropyl celluloses are soluble both in water and certain organic solvents. Hypromellose can be substituted by both methyl and hydroxypropyl groups, thus allowing fine-tuning of properties for applications such as use in hydrophilic matrix tablets (see FIG. 1).

Hypromellose concentration is an important consideration in the design of a controlled release hydrophilic matrix tablet. The hypromellose concentration must be high enough to ensure that a continuous gel layer is formed immediately upon exposure to an aqueous medium. Once such a concentration has been exceeded, however, an increase in hypromellose concentration will lead to a decrease in release rate due to an increase in the time required for the hypromellose to disentangle at the tablet surface. At some point, the disentanglement effect will reach a plateau such that a further increase in hypromellose concentration will not result in further reduction of drug release rate. This is because drug release does not result solely from hypromellose erosion, but also from diffusion of solubilized drug within the hydrated matrix. The precise position of the lower concentration threshold and upper plateau concentration will depend upon the characteristics and loading of the drug and other excipients, but in general the hypromellose concentration must lie in the range 20% to 50%.

Hypromelloses may be characterized by the following parameters:

Degree of substitution (“DS”). DS refers to the level of substitution in terms of the number of substituted hydroxyl groups, regardless of the nature of the substituent group, expressed as an average. For hypromellose, DS is usually redefined to reflect only methoxyl substitution. In either case the total available hydroxyl groups number 3, so DS lies between 0 and 3, but is most typically between 1.3 and 2.6.

Molar substitution (“MS”). For hypromellose, MS refers to the extent of hydroxypropyl substitution in terms of moles per mole of anhydroglucose, and is expressed as an average. Typical values lie in the range 0.2-0.4. Because each hydroxypropyl group contains a hydroxy group, there is no theoretical upper limit for MS.

Assay. Assay refers to methoxy (—OCH₃) and hydroxypropoxy (—OCH₂CHOHCH₃) content, expressed as a percentage.

Chemistry. Chemistry is defined by the assay values and is important in determining the hydrophilicity and hence the solubility of the hypromellose. Hypromellose that is sold under the trademark METHOCEL® (The Dow Chemical Company, Michigan, USA) is available in four established grades that are differentiated by chemistry, as shown in Table 12.

TABLE 12

| | | Nominal | Nominal |
|---|---|---|---|
| | | methoxy | hydroxypropoxy |
| Trade name | Compendial name | content | content |
| METHOCEL ® A/ | methylcellulose | 29% | 0% |
| Metolose ™ SM | | | |
| METHOCEL ® K/ | Hypromellose | 22% | 8% |
| Metolose ™ 90SH | 2208 | | |
| METHOCEL ® E/ | Hypromellose | 29% | 10% |
| Metolose ™ 60SH | 2910 | | |
| METHOCEL ® F/ | Hypromellose | 29% | 6% |
| Metolose ™ 65SH | 2906 | | |

For a controlled release matrix tablet formulation, a fast rate of hydration/gelation for the rate-controlling polymer, such as hypromellose, can provide the formulation a protective layer around the tablet core. The hydration rates of the various grades of hypromellose differ due to the difference in chemistry. It has been postulated that a hydroxypropyl group acts as a hydrophilic substituent that greatly contributes to the rate of hydration, whereas a methoxyl group is relatively hydrophobic and does not contribute to the rate of hydration. Thus, the rate of hydration of the different hypromellose chemistries is considered to depend upon the ratio of hydroxypropoxyl to methoxyl substitution, the higher ratio chemistries exhibiting more rapid hydration/gelation. Hence, K and E chemistry products are most commonly used in controlled-release matrix tablets.

The hydroxypropoxy and methoxy content of hypromellose are most commonly measured using a modification of the Zeisel alkoxy reaction, which uses a hydriodic acid treatment followed by gas chromatographic determination of the liberated methyl and isopropyl iodides (see, e.g., The United States Pharmacopoeia (USP30-NF25), United States Pharmacopoeia Convention, Inc., 2007, p. 2323 and DOW Analytical Method DOWM 100755-ME00B, The Dow Chemical Company, 2002). Sample preparation is time consuming, involves the use of hazardous reagents at elevated temperature and pressure, and requires careful control if meaningful results are to be achieved.

Proton Nuclear Magnetic Resonance spectrometry (1H NMR) has been used to measure hydroxypropoxy content of O-(2-hydroxypropyl)cellulose (see, e.g., Determination of substituent distribution in cellulose ethers by 13C- and 1H-NMR studies of their acetylated derivatives: O-(3-hydroxypropyl)cellulose, Tezuka, Y.; Imai, K.; Oshima, M. and Ciba, T., Carbohydr. Res. 196, 1 (1990)). A similar procedure, involving the preparation of an acetyl derivative of the intact polymer to confer solubility in NMR solvents across a wide range of substitution, has been developed for hypromellose (see, e.g., NMR Method 1, below). This method demonstrates superior precision to the USP method, but sample preparation is time-consuming (the acetylation reaction takes 3 days). The determination of hydroxypropoxy content of hydroxypropyl cellulose without derivatisation, using deuterated chloroform as solvent, has been described (see, e.g., Determination of molar substitution and degree of substitution of hydroxypropyl cellulose by nuclear magnetic resonance spectrometry. Ho, F. F.-L., Kohler, R. R., Ward, G. A., Anal. Chem. 44, 178 (1972)); however, a recent evaluation of this procedure showed poor reproducibility (see, e.g., Determination of the hydroxypropoxy content in hydroxypropyl cellulose by 1H NMR. Andersson, T., Richardson, S., Erikson, M., Pharmeuropa 15, 271 (2003)). Further work has been carried out to develop a method to determine the hydroxypropoxy and methoxy content of underivatised Hypromellose, using D2O/DMSO solvent, which is suitable for routine use (see NMR Method 2, below).

NMR Method 1. The degree of substitution is indirectly determined on acetylated samples with proton nuclear magnetic resonance (1H NMR). Acetylation of the samples is carried out by dissolving 75 mg of each of the polymer samples in 2.25 ml acetic anhydride and 0.75 ml pyridine. The solutions are heated up to 90° C. under stirring for 6 hours and are then dialyzed against deionised water in a Spectra/Por dialysis membrane (with molar mass cut off on 10 kDa) for 24 hours. The samples are dried before dissolution in deuterated chloroform (0.8 mg/ml). The 1H NMR spectra are recorded on a Varian 500 Inova spectrometer (USA) operating at a magnetic field of 11.7 T and equipped with a 5 mm 1H inverse detecting gradient probe. The free induction decay is recorded with at least 16 scans and the spectral window is between −1 and 16 ppm, referring to the solvent signal of CDCl3. Spectra are recorded at 50° C. The weight percentage of methoxy (MeO) groups and hydroxypropyl (HP) groups are calculated accordingly to the following formula:

$$MeO \% = \frac{( 31 \cdot DS \cdot 100 )}{( 58 \cdot MS + 162 + 14 \cdot DS )}$$

$$HP \% = \frac{( 75 \cdot MS \cdot 100 )}{( 58 \cdot MS + 162 + 14 \cdot DS )}$$

where DS, degree of substitution, and MS, molar substitution, were achieved through the NMR spectra (see, e.g., Determination of the hydroxypropoxy content in hydroxypropyl cellulose by 1H NMR. Andersson, T., Richardson, S., Erikson, M., Pharmeuropa 15, 271 (2003)).

NMR Method 2. Hydroxypropoxy and methoxy content are directly determined by Nuclear Magnetic Resonance Spectrometry as follows. 3.5 to about 4.5 mg of hypromellose is dissolved in a solvent, which is 99.96% D2O. The hypromellose is heated at about 105° C. for about 30 minutes prior to dissolving in the solvent. The hypromellose is heated at about 80° C. for about 15 minutes after dissolving in the solvent. The nuclear magnetic resonance spectrometer comprises a 1H{X} inverse detection probe. The temperature is about 353K. The pulse is about 45°. The spectrum width is about −2.5 to 13.5 ppm. The pulse repetition is about 15 seconds. The exponential line broadening is about 1.0 Hz. The spectrum is referenced to residual dimethyl sulfoxide (DMSO) peak at 2.70 ppm. The baseline of the nuclear magnetic resonance spectrum is corrected. The number of scans is selected such that the signal:noise ratio at 200 Hz for the peak at 1.2 ppm is greater than 500. The number of time domain data points is about 65,000. The number of processed data points is about 250,000.

Table 13 shows hydroxypropoxy (“HP”) and methoxy (“MeO”) contents, expressed as weight-percent of 18 solid dosage forms of a formulation, as determined using the United States Pharmacopoeia (“USP”) method, NMR Method 1 and NMR Method 2.

TABLE 13

| | | NMR (1) HP | NMR (2) HP | | NMR (1) MeO | NMR (2) MeO |
|---|---|---|---|---|---|---|
| Batch Reference | USP HP (%) | (%) | (%) | USP MeO (%) | (%) | (%) |
| (a) | 8.3 | 10.1 | 9.8 | 23.9 | 25.3 | 28.5 |
| (b) | 8.0 | 10.2 | 9.9 | 23.9 | 25.5 | 28.8 |
| (c) | 8.8 | 10.7 | 10.4 | 23.1 | 24.9 | 27.4 |
| (d) | 8.8 | 10.9 | 10.5 | 23.6 | 26.0 | 28.2 |
| (e) | 8.7 | 10.9 | 10.5 | 24.3 | 25.9 | 29.0 |
| (f) | 9.0 | 10.9 | 10.6 | 23.8 | 25.7 | 28.4 |
| (g) | 8.7 | 10.5 | 10.6 | 23.6 | 25.8 | 29.0 |
| (h) | 8.8 | 10.9 | 10.6 | 22.9 | 24.8 | 27.5 |
| (i) | 8.6 | 11.2 | 10.6 | 23.4 | 25.7 | 28.2 |
| (j) | 8.9 | 10.8 | 10.6 | 24.1 | 26.0 | 29.0 |
| (k) | 8.8 | 10.8 | 10.6 | 24.0 | 26.1 | 29.2 |
| (l) | 8.7 | 11.2 | 10.7 | 23.5 | 25.7 | 28.5 |
| (m) | 8.7 | 11.5 | 10.7 | 23.6 | 25.7 | 28.5 |
| (n) | 9.0 | 11.2 | 10.7 | 23.6 | 25.7 | 28.7 |
| (o) | 9.0 | 11.3 | 10.8 | 22.9 | 24.8 | 27.6 |
| (p) | 9.1 | 11.1 | 11.2 | 22.7 | 25.8 | 27.9 |
| (q) | 10.0 | 12.6 | 11.8 | 23.4 | 25.3 | 26.4 |
| (r) | 10.9 | 13.7 | 13.4 | 24.3 | 26.5 | 29.1 |
| Intermediate | 1.5 | 1.1 | 0.6 | 1.0 | 0.5 | 1.5 |
| precision | | | | | | |
| (% RSD) | | | | | | |
| Reproducibility | Not | 1.9 | 6.4 | Not | 0.6 | 10.3 |
| (% RSD) | available | | | available | | |

Multivariate analysis identified hypromellose hydroxypropoxy content to be the most important uncontrolled factor in determining the release of active ingredient from the formulations. FIG. 8 shows results of multivariate analysis that identified the hydroxypropyl content of the low- and high-viscosity USP 2208-chemistry hypromellose to be the most important uncontrolled factors affecting release of active ingredient from solid dosage forms of the formulations. The vertical axis shows Variable Influence on Projection, VIP, which is a measure of the relative importance of the factors, listed on the horizontal axis, that may affect release. (see, e.g., PLS. Wold, S., Johansson, E., Cocchi, M. in 3D-QSAR in Drug Design, Theory, Methods and Applications. Kubinyi, H., (ed.), ESCOM Science, Ledien, pp 523-550, 1993).

The factors, in the order shown in FIG. 8, are: Polymer Ratio (controlled factor used to compensate for variation in hypromellose lot characteristics), low-viscosity hypromellose hydroxypropoxy content (“100 cP HP”), high-viscosity hypromellose hydroxypropoxy content (“4000 cP HP”), high-viscosity hypromellose number average molecular weight (“4000 cP Mn”), high-viscosity hypromellose weight average molecular weight (“4000 cP Mw”), Low-viscosity hypromellose viscosity (“100 cP Viscosity”), low-viscosity hypromellose methoxy content (“100 cP MeO”), high-viscosity hypromellose %-through-100-mesh (“4000 cP 100 mesh”), low-viscosity hypromellose average particle diameter (“100 cP PS D50”), low-viscosity hypromellose weight average molecular weight (“100 cP Mw”), low-viscosity hypromellose %-through-100-mesh (“100 cP 100 mesh”), high-viscosity hypromellose average particle diameter (“4000 cP PS D50”), low-viscosity hypromellose number average molecular weight (“100 cP Mn”), high-viscosity hypromellose methoxy content (“4000 cP MeO”), low-viscosity hypromellose particle size span (“100 cP PS Span”), high-viscosity hypromellose particle size span (“4000 cP PS Span”), and high-viscosity hypromellose viscosity (“4000 cP Viscosity”).

Given the importance of hydroxypropoxy content it is important to use the best possible test method. NMR method 2, while less robust than NMR Method 1 (particularly with regard to transfer between laboratories), has been optimised for hydroxypropoxy determination and is considered suitable for routine operation by a skilled operator in one location. NMR Method 1 is useful as a reference method or where operation on multiple sites is a requirement, whereas the USP method is suitable to determine comformance to pharmacopoeial standards but is considered to be too variable to be used in isolation as a tool for hypromellose lot selection. Accordingly, except where otherwise specified, NMR characterization of HPMC refers to NMR Method 2.

Cloud Point. Aqueous solutions of hypromellose undergo a phenomenon known as thermal gelation, whereby upon heating gelation will occur at a specific temperature determined by the hypromellose chemistry and solution concentration. This effect is attributed to a gradual loss of water of hydration as temperature increases, reflected by a gradual decrease in viscosity. Once dehydration has reached a critical point, hydrophobic (polymer-polymer) interactions predominate, leading to an expansive network structure and a sharp increase in viscosity. The temperature at which light transmissivity reaches 50% its original value is termed the cloud point. The onset of gelation may also be measured (temperature at 95% transmission) as can a complete temperature—transmission profile.

An illustrative protocol for determining cloud point is as follows: 50 mL citric acid (0.05M/sodium hydroxide (0.09M) buffer (pH 4.70-4.90) in a 100 mL container is heated to 75±5° C. and 500±2 mg of the hypromellose test sample is added with rapid stirring. Stirring is continued for approximately 5 minutes to ensure complete dispersion. The container is transferred to an ice bath and slow stirring is continued for an additional 20 minutes. The resulting solution is then refrigerated overnight to ensure complete dissolution.

Cloud point is measured using a Cloud Point Analyser, such as the Mettler-Toledo FP900 Thermosystem comprising a Mettler-Toledo FP90 central processor and a Mettler-Toledo FP81C clear and cloud point measuring cell. Sample capillaries (Fisher part number UC-18572 or equivalent) are filled with sample solution to a height of approximately 10 mm using a Pasteur pipette, taking care to avoid entrapment of air, and placed in the measuring cell. Light transmittance is measured continuously while the samples are heated over the temperature range 40-80° C. at a rate of 1° C. per minute with a waiting time of 30 s. Each test is performed in triplicate and the average values for Tcp96 (the temperature at which light transmittance is 96% of the value at 40° C.) and Tcp50 (the temperature at which light transmittance is 50% of the value at 40° C.) were recorded.

Table 14 shows cloud point measurements for 16 solid dosage forms of a formulation having hydroxypropoxy content in the range 10.2-13.7%.

TABLE 14

| Batch | | | Hydroxypropoxy content |
|---|---|---|---|
| Reference | Tcp96 (° C.) | Tcp50 (° C.) | by NMR Method 1 (%) |
| 1 | 60.8 | 64.2 | 13.7 |
| 2 | 61.8 | 66.0 | 11.5 |
| 3 | 61.7 | 66.1 | 11.2 |
| 4 | 62.4 | 66.4 | 11.2 |
| 5 | 59.7 | 66.5 | 12.6 |
| 6 | 63.4 | 67.5 | 10.8 |
| 7 | 63.9 | 67.5 | 11.2 |
| 9 | 63.8 | 67.8 | 10.9 |
| 8 | 64.2 | 67.9 | 10.8 |
| 10 | 62.4 | 68.1 | 10.9 |
| 11 | 64.3 | 68.3 | 10.9 |
| 12 | 65.1 | 68.9 | 10.9 |
| 13 | 66.2 | 70.2 | 11.3 |
| 14 | 66.3 | 70.4 | 10.1 |
| 15 | 63.4 | 70.8 | 10.7 |
| 16 | 66.8 | 71.3 | 10.2 |

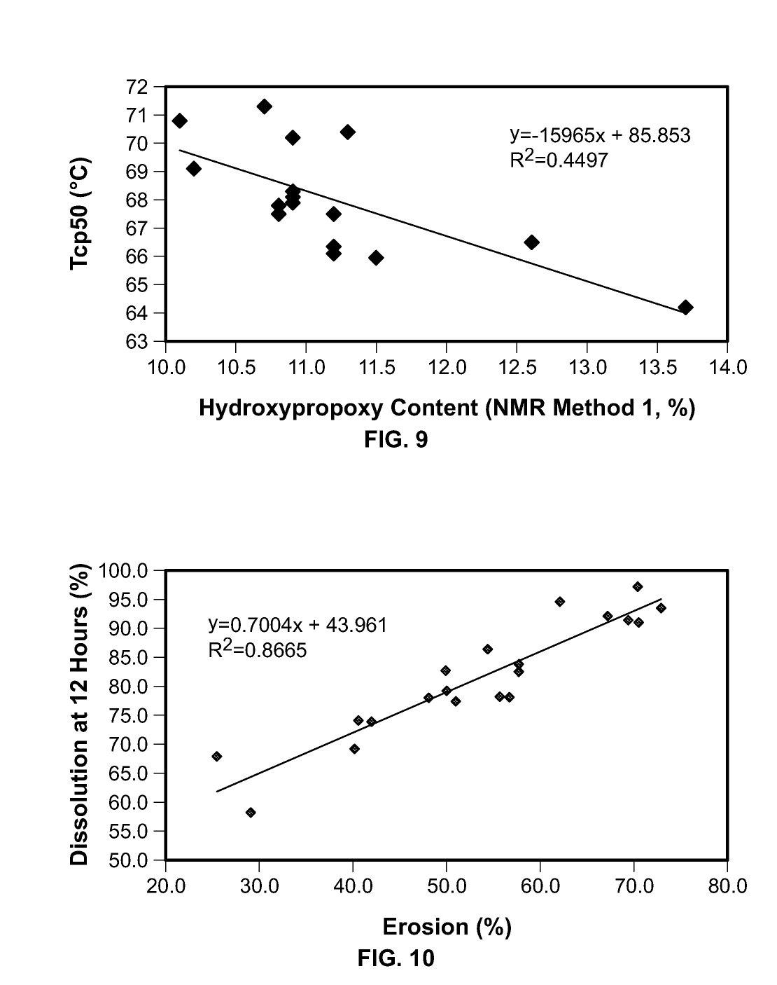

FIG. 9 shows, based on the data shown in Table 14, a weak correlation between cloud point and hydroxypropoxy content.

Because cloud point is related to hypromellose hydrophilicity, a property that depends largely on the extent of hydroxypropoxy and methoxy substitution, it is possible that cloud point may be useful as an active ingredient release factor, acting as a surrogate for the more complex and costly NMR methods.

Viscosity. The viscosity of a 2% (weight hypromellose/weight water) solution of hypromellose in water may be measured by Ubbelohde viscosimeter and expressed in centipoise (cp). Further information can be found in C. M. Keary, Characterization of METHOCEL cellulose ethers by aqueous SEC with multiple detectors, Carbohydrate Polymers 45 (2001) 293-303, which is hereby incorporated by reference herein in its entirety.

The viscosity and %-through-100-mesh are determined by hypromellose suppliers (e.g., Dow Chemical and Shin-Etsu Chemical Companies). Viscosity may be determined using a U.S. Pharmacopoeia hypromellose monograph method.

Erosion. Solid dosage forms may release active ingredient by hypromellose compact erosion, which may be measured as follows. Compacts of hypromellose, which may include Methocel K100 and Metolose SR [Type 90SH] (Hypromellose 2208 USP, 100 cP), are prepared by direct compression. The hypromellose is mixed with magnesium stearate (1.5%) in a small V-blender for 2 minutes. Compacts are prepared using a F-press (0.3×0.748″ shaped tooling) to a target weight of 640 mg (±10 mg) and a target hardness of 20-25 Kp. Verification of consistent weights and hardness values is conducted by determining the weight and hardness of 5 individual compacts before running the press and once the press was started random samples are taken to ensure consistency.

Erosion studies may be performed in triplicate using an USP I basket apparatus in 0.05 M citric acid/0.09 M NaOH pH 4.8 buffer (900 mL) maintained at 37° C. and agitated at a speed of 100 rpm. Each compact is weighed before starting the test. The baskets are removed from the medium at 16 hours and dried at 60° C. in an oven for a 24 hour period. The residues are then cooled over desiccant before weighing.

The erosion percentage was calculated as follows:

% Erosion=(W₁−W₂)*100/(W₁),

in which W1 is compact weight before testing and W2 is cooled residue weight.

Table 15 shows percent erosion for 20 solid dosage forms of a formulation.

TABLE 15

| | | Dissolution of active |
|---|---|---|
| Batch | Erosion (%)after 16 | ingredient at 12 hours |
| Reference | hours | (%) |
| A | 29.1 | 58.2 |
| B | 25.5 | 67.9 |
| C | 40.2 | 69.2 |
| D | 42.0 | 73.9 |
| E | 40.6 | 74.1 |
| F | 51.0 | 77.4 |
| G | 48.1 | 78.0 |
| H | 56.7 | 78.1 |
| I | 55.7 | 78.2 |
| J | 50.0 | 79.2 |
| K | 57.7 | 82.5 |
| L | 49.9 | 82.7 |
| M | 57.7 | 83.8 |
| N | 54.4 | 86.4 |
| O | 70.5 | 91.0 |
| P | 69.4 | 91.4 |
| Q | 67.2 | 92.1 |
| R | 72.9 | 93.5 |
| S | 62.1 | 94.6 |
| T | 70.4 | 97.2 |

Based on the data in Table 15, there is a strong correlation between the rate of active ingredient release from solid dosage forms containing low viscosity hypromellose (along with high viscosity hypromellose and other excipients) and the erosion of compacts of low viscosity hypromellose, as exemplified in FIG. 10 for the 12-hour dissolution time-point.

Thus, the erosion test could be used as a performance test in the evaluation of new lots of low viscosity hypromellose, either to identify and reject those lots which which would lead to tablets with unacceptable drug release characteristics, or to determine an appropriate ratio of low- to high-viscosity hypromellose which would lead to tablets having acceptablerelease characteristics.

Particle size. Particle size may be measured by air-jet sieving.

Thus, commercially available hypromellose products may be classified in terms of chemistry (methoxy and hydroxypropoxy content), viscosity and physical form (particle size). In the case of METHOCEL products, the classification takes the following form: METHOCEL X NY P, where X identifies the hypromellose as E, F, or K; NY indicates the viscosity (N being a number and Y, if present, a letter indicating a multiplier, “IC” representing 100 and “M” representing 1000, the multiplicative product being apparent viscosity in mPa·s, 2% solution in H₂O at 20° C.); P is a suffix that, if present, may be used to identify special products (“LV” refers to low viscosity, “CR” to a controlled-release grade, “EP” to a product that meets the requirements of the European Pharmacopoeia, and so forth).

A buffering agent (such as sodium citrate dihydrate) may increase pH within a hydrated tablet core, thus decreasing core solubility to minimize diffusive release. For the formulations, the selection of lactose, microcrystalline cellulose and magnesium stearate was conducted in accordance with industry practice. Formulations for different tablet strengths are shown in Table 16.

TABLE 16

| | Tablet Formulation | | | | |
|---|---|---|---|---|---|
| | 400 mg | 300 mg | 200 mg | 150 mg | 50 mg |
| Ingredients | | | | | |
| Quetiapine fumarate | 460.50 | 345.38 | 230.26 | 172.69 | 57.56 |
| Lactose mono-hydrate | 15.50 | 49.31 | 52.87 | 74.65 | 125.72 |
| Microcrystalline | 15.60 | 49.31 | 52.87 | 74.56 | 125.72 |
| cellulose | | | | | |
| Sodium citrate Dihydrate | 100.00 | 100.00 | 75.00 | 71.88 | 36.00 |
| hypromellose 2208 | 234.90 | 200.00 | 138.00 | 120.75 | 120.00 |
| 100 cp | | | | | |
| hypromellose 2208 | 26.10 | 40.00 | 42.00 | 51.75 | 30.00 |
| 4000 cp | | | | | |
| Magnesium stearate | 17.40 | 16.00 | 9.00 | 8.63 | 5.00 |
| Purified water | qs | qs | qs | qs | qs |
| Total Tablet Weight | 870.00 | 800.00 | 600.00 | 575.00 | 500.00 |
| Coating materials | | | | | |
| HPMC 2910, 6 cps | 0 | 11.765 | 8.82 | 0 | 7.353 |
| Polyethylene glycol | 2.726 | 3.529 | 2.65 | 1.802 | 2.206 |
| 400 NF | | | | | |
| Chromatone DDB-^{a} | 19.077 | 4.706 | 3.53 | 12.608 | 2.941 |
| Purified water | 123.6 | 180.0 | 135.0 | 81.7 | 112.5 |
| Total Coating Weight | 21.8 | 20.0 | 15.0 | 14.4 | 12.5 |

^{a}Pigment blends with luminosity and color indicated are as follows: SSR 400 mg: 8146W (white); SSR 300 mg: 8580Y (yellow); SSR 200 mg: 7757-Y (yellow); SSR 150 mg: 8146W (white); SSR 50 mg: 7756-OR (orange).

Investigation revealed tablet dissolution variability within a batch of tablets that could not be attributed to any single factor, but depended upon four hypromellose factors: viscosity/molecular weight, particle size distribution, hydroxpropoxyl content, and methoxyl content. The relative importance of these factors was found to vary depending on tablet strength, and hypromellose from different suppliers (e.g., Dow Chemical Company and Shin-Etsu, Ltd.) was found to behave differently.

An increase in viscosity (an increase in chain length and hence molecular weight) leads to a reduction in the rate of surface erosion and hence in the rate of drug release. There is some evidence that this effect may plateau at high viscosities. The blending of high- and low-viscosity hypromellose to achieve intermediate viscosity may be modeled using the Phillip of equation: η=(1+KC)⁸, where η=viscosity in cp, K=a constant for each individual polymer batch, and C=concentration expressed as a percentage. Formulations that include combinations of hypromellose grades may be susceptible to variations in viscosity that may occur as a result of within-specification variability of hypromellose batches.

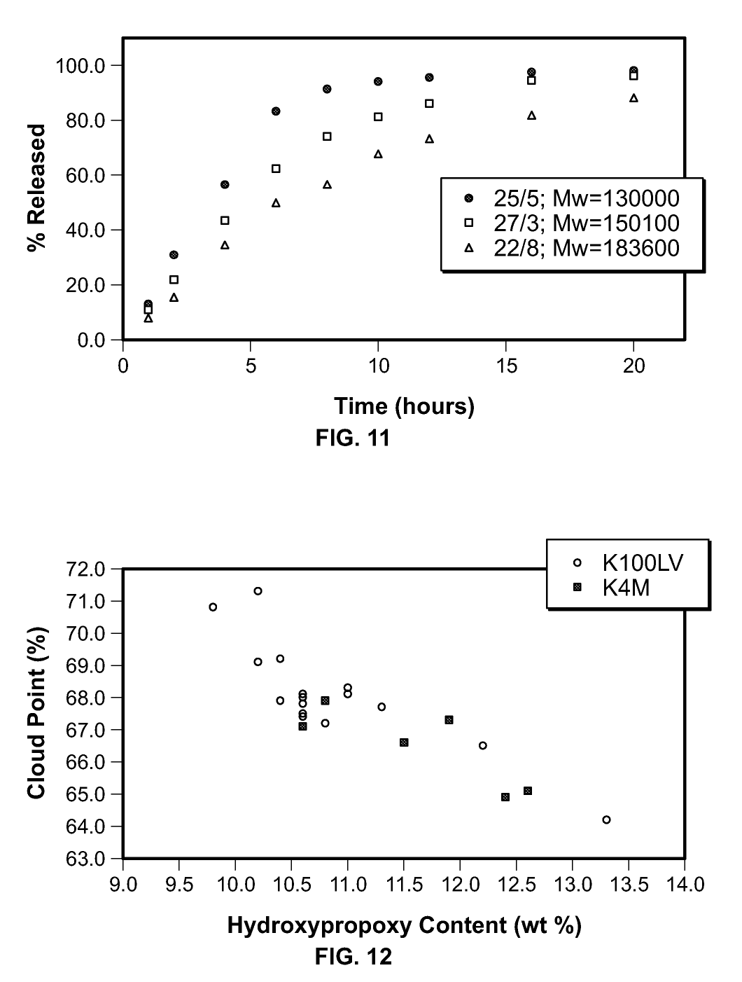

The effect of deliberate variation of viscosity brought about by adjusting the proportions of low- and high-viscosity grades of hypromellose 2208, as characterized by weight average molecular weight (Mw), for three batches of tablets, is shown in FIG. 11.

Smaller particles that have a greater surface area:mass ratio hydrate more rapidly than larger particles. This leads to more effective formation of the protective gel barrier. In contrast, tablets manufactured from larger particles of hypromellose tend to disintegrate. This leads to rapid and uncontrolled release of drug.

With regard to hydroxypropoxyl and methoxyl content, the formulation and methods of preparation are based on theories that are at odds with widely accepted assumptions about hypromellose matrix chemistry (see, e.g., Using Dow Excipients for Controlled Release of Drugs in Hydrophilic Matrix Systems, The Dow Chemical Company, Midland, Mich., 2006). It previously has been postulated, as mentioned before, that the hydroxypropyl group acts as a hydrophilic substituent that greatly contributes to the rate of hydration, whereas the methoxyl group acts as a relatively hydrophobic substituent and does not contribute to the rate of hydration. The rate of hydration of the different chemistries of hypromellose was therefore considered to depend upon the ratio of hydroxypropoxyl:methoxyl substitution.

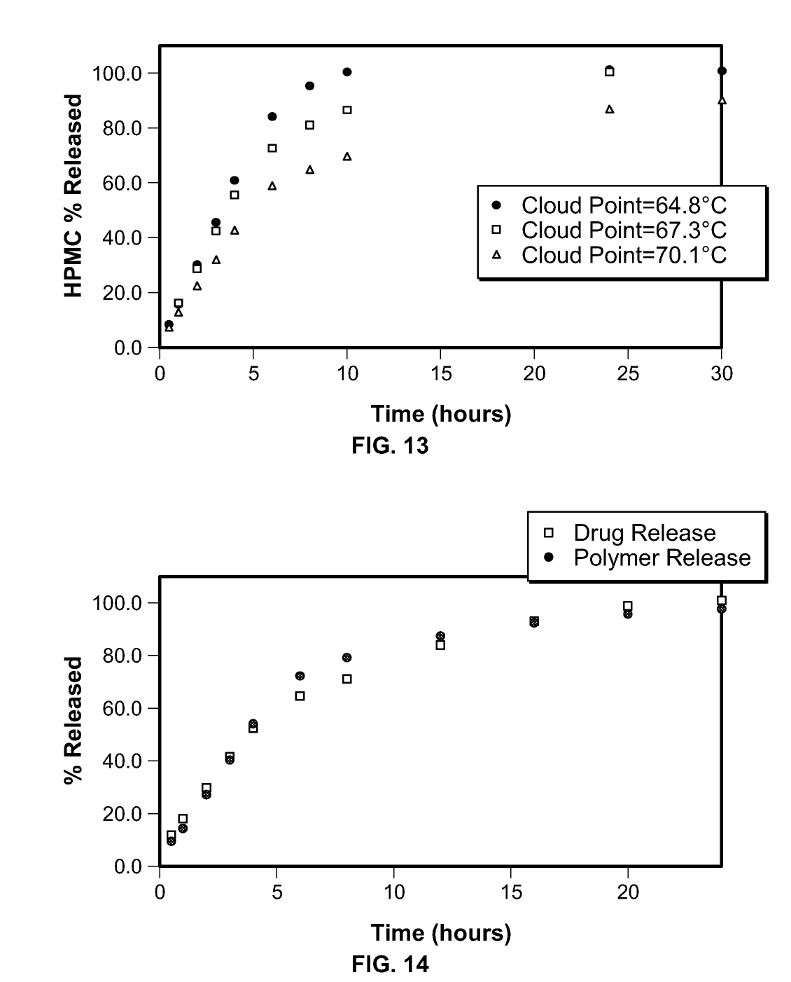

Contrary to this hypothesis, cloud point measurements have shown that, for the polymer batches studied, methoxyl and hydroxypropoxyl groups both act as hydrophobic substituents, such that an increase in the content of either leads to a decrease in cloud point. The inverse relationship between hydroxypropyl content and cloud point for hypromellose batches having a similar level of methoxy substitution is shown in FIG. 12. Furthermore, when hypromellose batches are used in the formulation, all other factors being equal, such a decrease in cloud point leads to an increase in drug release rate, as shown in FIG. 13. Studies into release mechanism have shown that quetiapine release from tablets is controlled exclusively by erosion, as illustrated by the coincident release profiles for quetiapine and hypromellose in FIG. 14. Thus, variations in methoxyl content and hydroxypropoxyl content affect the rate of erosion.

Methods of preparing a formulation comprise batch-wise variation in the ratio of a high- and low- viscosity hypromellose to offset the normal variations in hydroxypropoxyl content, methoxyl content, and viscosity of hypromellose batches, which would otherwise lead to unacceptable variability in the dissolution profile of quetiapine from tablets. The methods differ from the conventional “Master Formula” approach, wherein every batch of a formulation is prepared identically by dispensing the active ingredient and excipients in fixed quantities and processing them in an identical manner. In methods of the invention, the total hypromellose content may be fixed for all batches but the ratio of the low- and high-viscosity hypromellose may be different in different batches, among which the ratio may vary between 15.0:15.0 and 29.0:1.0.

The methods of the invention may involve laboratory procedures (e.g., hydroxypropoxyl measurement by nuclear magnetic resonance) that may have reduced variability in comparison to compendial test methods. The methods may involve predictive tools to determine the ratio of the high- and low-viscosity hypromellose batches to achieve a dissolution profile for a given strength formulation. The predictive tool may take the form of a look-up table (derived from historical data), a multivariate mathematical model, or any other suitable heuristic tool.

The methods may improve the frequency with which dosage forms satisfy drug release specifications for commercial products, support the use of a broad purchase specification for hypromellose batches in line with supplier capability, allow the use of hypromellose from different suppliers, support the use of different sites and scales of manufacture, and/or support the manufacture of dosage form batches having faster or slower release profiles, such as may be required for pharmacokinetic studies.

The methods may be applied to the foregoing formulations and to other formulations of quetiapine, or pharmaceutically acceptable salts thereof, or to formulations comprising other active substances and a hypromellose content between 15 and 55%.

Some embodiments of the invention comprise a multivariate model that may be used to correlate hypromellose properties and formulation information to in vitro measurements of tablet dissolution. It was determined that the hypromellose content and the viscosity of hypromellose contribute to the release rate of quetiapine from quetiapine extended release tablet formulations. Unexpectedly, not only do the hypromellose content and viscosity ratios impact release rates, but also the polymer properties [e.g., hydroxypropoxy content] impact release rates.

The model may be an artificial neural network (“ANN”) model, which may exhibit low prediction errors in comparison to other models. An ANN is a mathematical procedure for correlating variables with an output. The ANN develops a correlation between known inputs and known outputs in a process referred to as “training.” A multi-layered feedforward Neural Network (“NN”) was reported, for example, by Despagne, F. and D. Luc Massart, 1998, “Neural networks in multivariate calibration,” Analyst, 123:157R-178R, which is incorporated by reference herein in its entirety. A numerical analysis platform sold under the trademark MATLAB, which is available from The MathWorks, Inc. of Natick, Mass., is one commercially available tool for training neural networks and using defined neural networks for prediction. The feedforward NN and fast back-propagation are available through a number of commercially available software packages.

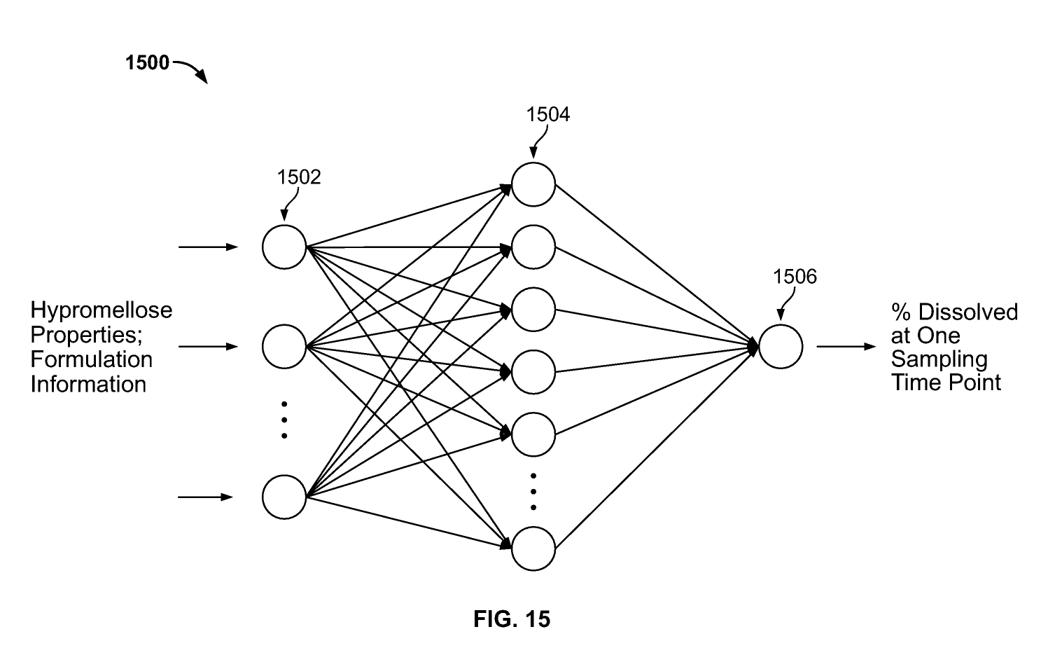

FIG. 15 shows a simplified representation of feedforward ANN 1500 with the inputs and outputs relevant to the formulations of the invention as described herein. FIG. 15 shows input layer 1502, hidden layer 1504, and output layer 1506. Hypromellose properties and formulation information are input to input layer 1502.

Output 1506 is % dissolved, i.e., the % quetiapine released for a single time point. The extended release dissolution curve of quetiapine tablets as described herein, and other pharmaceutically acceptable salts, may be modeled using one independent neural network per dissolution sampling time point. The results may be combined to give a dissolution profile that spans different time points.

An example of ANN architecture for quetiapine formulations as described herein and other pharmaceutically acceptable salts is set forth in Table 17. The items referred to in Table 17 along with the input parameters and dissolution results, define an ANN used for quetiapine tablets as described herein (and its pharmaceutically acceptable salts, more particularly the fumarate salt). For discussion of ANN architectures and the parameters shown in Table 17, see, e.g., Despagne and Massart, 1998 (cited above). Model inputs that may be relevant to the formulations are discussed herein, and other model inputs maybe used for other embodiments of the invention, e.g., embodiments of the invention that may be used for other pharmaceutical compositions.

TABLE 17

| | |
|---|---|
| ANN parameter | Multilayered feedforward |
| Training algorithm | Fast back-propagation |
| Input scaling | −1 to 1 |
| Number of hidden layers | 1 |
| Number of cells in hidden layer | 10 |
| Transfer functions - hidden and output layers | Hyperbolic tangent |
| Number of cells in output layer | 1 |
| Output | % quetiapine dissolved |

In some embodiments of the invention, there are two types of training information input into model 1500. The first type is information on the formulation, and the second type is data on specific hypromellose properties.

50 mg, 200 mg, 300 mg and 400 mg tablet strengths were included in the training of model 1500. Tablets were made according to the protocol set forth in Example 2 below. Formulation ingredients and tablet weights were included as inputs (see Table 18). Quantitative composition of ingredients was expressed as the relative content (weight %) of each ingredient for each tablet strength. For each batch of any given strength tablet, the only differences in the formulation inputs were the amounts of 100 cp and 4000 cp hypromellose, although total the sum of 100 cp and 4000 cp hypromellose was for each batch 30% by weight of the formulation. All other formulation ingredients remained fixed for each formulation strength.

TABLE 18

Quetiapine fumarate (weight %)
Lactose monohydrate (weight %)
Micro crystalline cellulose (weight %)
Sodium citrate (weight %)
hypromellose, 100 cp (weight %)
hypromellose, 4000 cp (weight %)
Magnesium stearate (weight %)
Tablet weight (mg)

Table 16 shows quantitative composition of tablets of quetiapine formulations as described herein and other pharmaceutically acceptable salts of different weights.

The second type of training information input into model 1500 was data on hypromellose properties. While commercially available data to showed compliance to pharmacopoeial standards, such data alone proved inadequate for understanding the correlation between hypromellose and dissolution results.

Eight hypromellose properties were selected for the model (see Table 19). Values for both the 100 cp and 4000 cp hypromellose for each property were included in the model.

TABLE 19

| Hypromellose property | Abbreviated as |
|---|---|
| Hydroxypropxy content (wt %) | % HP |
| Methoxy content (wt %) | % MeO |
| Viscosity (cp) | |
| Molecular weight: | |
| Weight average | MWw |
| Number average | MWn |
| Particle size: through 100 mesh (150 μm) | % <150 μm |
| Average particle diameter, 50% volume | D50 |
| distribution (μm) | |
| Particle size span | Span |

Hydroxypropoxy and methoxy content may be determined by a nuclear magnetic resonance spectrometry protocol such as NMR Method 2.

Values for viscosity and particle size (%-through-100-mesh) may be taken directly from the supplier's certificates of analysis and used in the model.

The average particle diameter and particle size span may be determined using a laser diffraction technique on the dry powder.

The number average molecular weight (Mn) and weight average molecular weight (Mw) are determined using an aqueous SEC method employing on-line light scattering detection for the direct determination of molecular weight. The units are Daltons.

The inputs and outputs in ANN model training data were mean-centered and range-scaled. By scaling, the maxima of the absolute value of the mean-centered inputs were set to the value one and the maxima of the absolute values of the mean-centered outputs were set to the values 0.5, 0.5, 0.5, 0.5, 0.5, 0.5, 0.8, and 0.85 respectively.

Weights and biases were initialized with small random numbers between −0.05 and 0.05.

A backpropagation algorithm that uses momentum and an adaptive learning rate is described below. The algorithm is discussed by Martin T. Hagan, Howard B. Demuth, and Mark Beale, Neural Network Design, Boston: PWS Publishing Co., 1996, which is hereby incorporated by reference herein in its entirety, and is summarized below.

During the training process, the weights and biases are adjusted according to the following formulas (some terms of which are more general than the corresponding terms that appear below in connection with the trained model):

ΔWᵢⱼ(t)=γΔWᵢⱼ(t−1)−(1−γ)λδᵢpⱼ

Δbᵢⱼ(t)=γΔbᵢⱼ(t−1)−(1−γ)λδᵢ

where δ is the learning rate, γ is the momentum factor, δᵢ is the correction term that is calculated using standard error backpropagation, pⱼ is the input at a neuron, and t represents the time sequence of the training process.

The following rules were used to adapt learning rate α during the training process. The rules involve calculating a squared error, which may be the squared error of one individual prediction, the summation of the squared errors of individual predictions in a training batch, or any other suitable measure of error between predicted and actual dissolution.

(1) If the squared error increases by more than 4% after a weight update, the weight update is discarded, the learning rate is multiplied by 0.7, and the momentum factor is set to zero;

(2) If the squared error decreases after a weight update, the weight update is accepted, and the learning rate is multiplied by 1.05. If the momentum factor has been set to zero previously, it is reset to its original value; (3) If the squared error increases by less than 4% after a weight update, the weight update is accepted. The learning rate and the momentum factor maintain the same values.

The training was stopped when 400 training epochs or a sum-squared error goal of 0.001 was reached. The initial learning rate was set to 0.01 and the size of the training batch was set to 10.

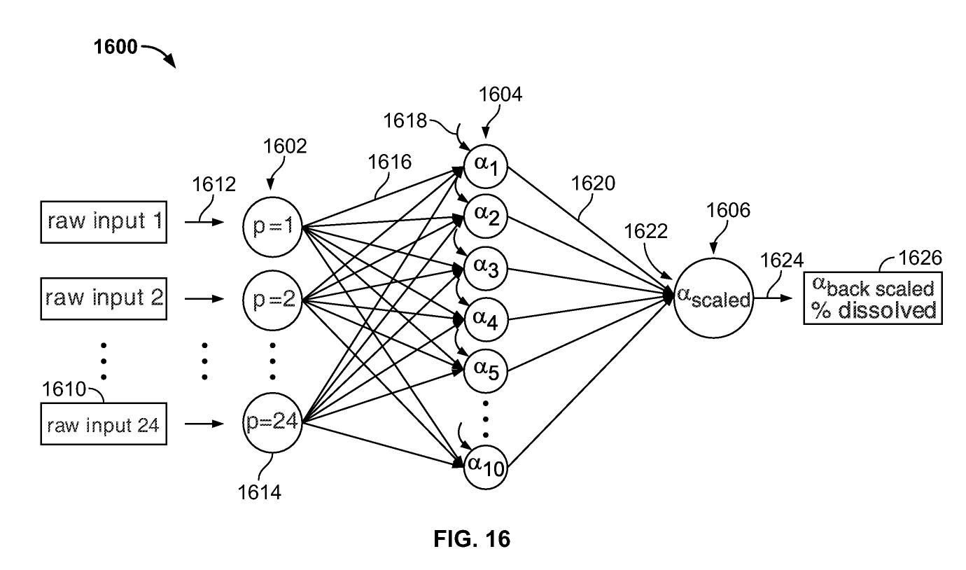

Model 1500 was trained using a training data set of 177 batches of formulations as described herein. Tablets of all strengths, two different commercial sources of hypromellose, development and commercial scale manufactures, and three manufacturing plants were used to train the model. The tablets included ratios of hypromellose 100 cp to 4000 cp ranging from 15:15 to 29:1 (%−100 cp: %−4000 cp). The ratios are also included in the model. Model 1600 (see FIG. 16) is an illustrative trained prediction model based on the model architecture shown in FIG. 15 and the training data set, which inherently reflects features of manufacturing equipment that may differ among manufacturers and manufacturing plants. Model 1600, therefore, may not predict dissolution behavior of tablets produced using equipment that is different from the equipment used to produce the tablets described herein. Nonetheless, model 1600 was trainable to tablets from different manufacturing processes, thus demonstrating that the ANN approach has general applicability, but models should be trained on the same equipment that is to be used for commercial production. A safeguard against over-fitting is to use the simplest ANN possible to fit the data. Model 1500 is considered an appropriately simple ANN architecture, because it contains a single hidden layer with only 10 cells.

Training was achieved by obtaining measurements of hypromellose lot physical and chemical properties, inputting the measurements into the model, predicting dissolutions, comparing predicted dissolution to in vitro dissolution of batch tablets made from the lots, and readjusting model constants until the model predictions were acceptable. The protocol for the in vitro dissolution assay is set forth in Example 7. The predicted dissolution profile may be compared to an actual tablet dissolution profile by calculating the root-mean-square error of prediction (“RMSEP”). The lower the RMSEP, the better the agreement between the actual and predicted profiles.

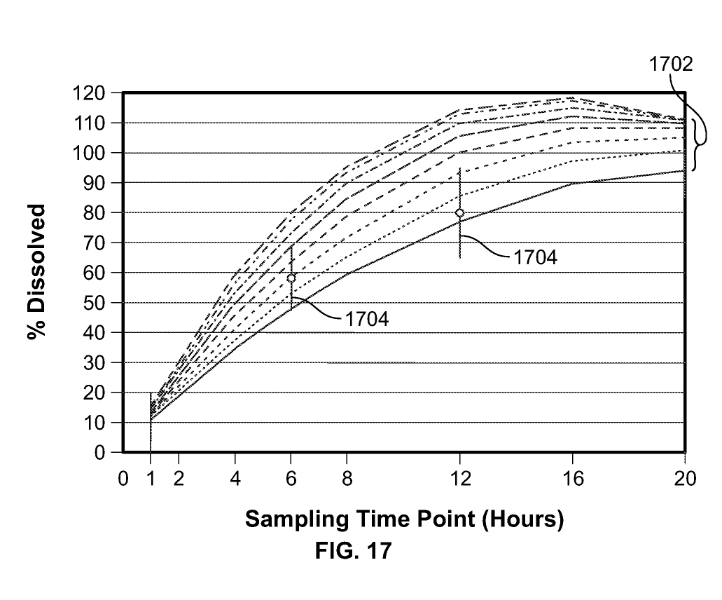

For 100 cp and 4000 cp hypromellose lots, model 1500 may be used to predict dissolution profiles for hypromellose ratios from 15:15 to 29:1 (100 cp:4000 cp) in ratio increments of 0.1 (e.g., 15.0:15.0, 15.1:14.9, 15.2:14.8, etc). FIG. 17 shows a range of curves 1702 that may include many predicted profiles corresponding to the incremental ratios. An optimal profile, and thus an optimal ratio, is identified by comparing the predicted dissolution results to the midpoints in the dissolution acceptance criteria ranges (bars 1704, FIG. 17) at 2 time points, 6 and 12 hours. A comparison of the predicted results to the midpoints is made by calculating a combined relative distance factor, d, using the equation:

$$d = \sqrt{\frac{{[ ( {p}_{6} - {c}_{6} ) \frac{{r}_{6} + {r}_{12}}{{r}_{6}} ]}^{2} + {[ ( {p}_{12} - {c}_{12} ) \frac{{r}_{6} + {r}_{12}}{{r}_{12}} ]}^{2}}{2}}$$

where:

P₆ is the predicted % quetiapine dissolved at the 6 hour time point;

C₆ is the % quetiapine dissolved at the midpoint in the dissolution acceptance criteria range at the 6-hour time point;

R₆ is acceptance criteria range in % quetiapine dissolved at 6 hours;

R₁₂ is acceptance criteria range in % quetiapine dissolved at 12 hours;

P₁₂ is the predicted % quetiapine dissolved at the 12 hour time point;

C₁₂ is the % quetiapine dissolved at the midpoint in the dissolution acceptance criteria range at the 12-hour time point.

The optimal ratio is identified by selecting the profile with lowest value of d.

Since the slope of the dissolution profile changes with the particular properties of hypromelloses used, often times the profile at the identified optimal ratio may not go through acceptance criteria midpoints (as shown by bars 1704 in FIG. 17) at either 6 or 12 hours.

Details of the batch manufacture completed using the optimal ratio determination are provided above.

Twenty-four raw inputs 1610 are scaled to conform to a range of −1 to +1 by respective scaling factors 1612. Scaled inputs 1614 are input into input layer 1602. Scaled inputs 1614 are transformed into 10 hidden layer 1604 values αⱼ (j=1 to 10) based on weights 1616 and biases 1618. Hidden layer 1604 values are transformed into output layer 1606 value α_{scaled} based on weights 1620 and biasₒᵤₜₚᵤₜ 1622. Value α_{scaled} is then scaled back to back-scaled output α_{backscaled} 1626 using scaling factor 1624.

Table 20 shows illustrative physical parameters of 24 raw inputs 1610 for model 1600. Raw inputs nos. 1-16 and 19-24 are based on empirical measurements, estimates or descriptive statistics of formulation parameters and hypromellose properties.

Raw inputs Nos. 17 and 18 are HPMC weight percents for 100 and 4000 cp HPMC, respectively. Taken together, raw inputs nos. 17 and 18 represent a ratio that is an independent variable to be optimized based on distance factor d. The sum of raw inputs nos. 17 and 18 is held constant at 30.0% and the ratios between raw inputs nos. 17 and 18 are varied in steps of 0.1 between 15.0:15.0 and 29.0:1.0.

TABLE 20

| Raw input | | Minimum | Maximum |
|---|---|---|---|
| number (P) | Physical parameter | Value | Value |
| 1 | 100 cp hydroxypropoxy Weight | 9.8 | 13.4 |
| | Percent | | |
| 2 | 4000 cp hydroxypropoxy Weight | 9.9 | 12.8 |
| | Percent | | |
| 3 | 100 cp methoxy Weight Percent | 26.4 | 29.2 |
| 4 | 4000 cp methoxy Weight Percent | 27.3 | 29 |
| 5 | 100 cp 100 mesh | 91.2 | 100 |
| 6 | 4000 cp 100 mesh | 90 | 96.6 |
| 7 | 100 cp Viscosity cp | 96 | 112 |
| 8 | 4000 cp Viscosity cp | 3684 | 5535 |
| 9 | 100 cp molecular weight | 123000 | 147000 |
| 10 | 4000 cp molecular weight | 304000 | 351000 |
| 11 | 100 cp molecular number | 38500 | 56500 |
| 12 | 4000 cp molecular number | 84000 | 140000 |
| 13 | 100 cp Particle Size D50 (μm) | 63.1 | 104.1 |
| 14 | 4000 cp Particle Size D50 (μm) | 55.3 | 107.6 |
| 15 | 100 cp Particle Size Span | 2 | 2.96 |
| 16 | 4000 cp Particle Size Span | 2.07 | 2.93 |
| 17 | 100 cp % | 15 | 29 |
| 18 | 4000 cp % | 1 | 15 |
| 19 | Quetiapine Fumarate % | 11.5 | 52.93 |
| 20 | Lactose Monohydrate % | 1.78 | 25.1 |
| 21 | Microcrystalline Cellulose % | 1.78 | 25.1 |
| 22 | Sodium Citrate % | 7.2 | 12.5 |
| 23 | Magnesium salt % | 1 | 2 |
| 24 | Tablet Weight | 500 | 870 |

Table 20 also shows the maximum and minimum values of each raw input physical parameter for which the model was trained and validated.

Table 21 shows the corresponding minimum and maximum values of scaled inputs 1614.

TABLE 21

| Raw and scaled | | |
|---|---|---|
| input number (P) | Minimum Scaled Value | Maximum Scaled Value |
| 1 | −0.416314737 | 1 |
| 2 | −1 | 0.980324074 |
| 3 | −1 | 0.631873559 |
| 4 | −1 | 0.629040117 |
| 5 | −0.513408473 | 1 |
| 6 | −1 | 0.978323455 |
| 7 | −0.861932939 | 1 |
| 8 | −1 | 0.827215232 |
| 9 | −0.750020598 | 1 |
| 10 | −1 | 0.909823458 |
| 11 | −0.674374606 | 1 |
| 12 | −0.746361746 | 1 |
| 13 | −0.392283637 | 1 |
| 14 | −1 | 0.712533531 |
| 15 | −1 | 0.755190579 |
| 16 | −0.746443323 | 1 |
| 17 | −1 | 0.742616034 |
| 18 | −0.742616034 | 1 |
| 19 | −1 | 0.853742821 |
| 20 | −0.779415949 | 1 |
| 21 | −0.779415949 | 1 |
| 22 | −1 | 0.555927818 |
| 23 | −1 | 0.863157895 |
| 24 | 0.773354996 | 1 |

Model 1600 may be run once for each pair of raw inputs nos. 17 and 18 for each of 8 time points to predict quetiapine fumarate %-dissolution 1626 (see FIG. 16) at six- and 12-hour time-points for the different ratios. The ratio that minimizes distance factor d may then be used as the ratio for production of the formulations described herein.

Scaled inputs 1614 may be determined using the following equation.

$${p}_{scaled} = \frac{p - xMean}{xScale} ,$$

where, for each raw input, p corresponds to a raw input 1610 and P_{scaled} corresponds to scaled input 1614. xMean and xScale for each raw input are set forth for exemplary model 1600 in Table 22.

TABLE 22

| Raw input no. | xMax | xMean | xScale |
|---|---|---|---|
| 1 | 1 | 10.8582 | 2.54181 |
| 2 | 1 | 11.3644 | 1.46441 |
| 3 | 1 | 28.1158 | 1.71582 |
| 4 | 1 | 28.3436 | 1.04356 |
| 5 | 1 | 94.1853 | 5.81469 |
| 6 | 1 | 93.3362 | 3.33616 |
| 7 | 1 | 103.407 | 8.59322 |
| 8 | 1 | 4697.02 | 1013.02 |
| 9 | 1 | 133286 | 13714.1 |
| 10 | 1 | 328610 | 24609.6 |
| 11 | 1 | 45749.7 | 10750.3 |
| 12 | 1 | 107933 | 32066.7 |
| 13 | 1 | 74.652 | 29.448 |
| 14 | 1 | 85.8395 | 30.5395 |
| 15 | 1 | 2.54695 | 0.546949 |
| 16 | 1 | 2.43757 | 0.492429 |
| 17 | 1 | 23.0339 | 8.0339 |
| 18 | 1 | 6.9661 | 8.0339 |
| 19 | 1 | 33.8494 | 22.3494 |
| 20 | 1 | 11.9946 | 13.1054 |
| 21 | 1 | 11.9946 | 13.1054 |
| 22 | 1 | 10.6063 | 3.40633 |
| 23 | 1 | 1.53672 | 0.536723 |
| 24 | 1 | 661.356 | 208.644 |

More generally, where raw inputs are represented by vector x, scaling may be performed as follows: For a given vector x (a column in the input data matrix), the mean of the vector (xMean) is first calculated, and x is then mean centered as below:

x_{mc}=x−xMean·I

where I is the identify vector. Next a predetermined xMax value (1 for all the raw inputs) may be used to calculate a scaling factor xScale using the following equation

$$xScale = \frac{max ( \vert {x}_{m c} \vert )}{xMax} .$$

The data may then be scaled using the following equation

$${x}_{scaled} = \frac{{x}_{m c}}{xScale} .$$

The output data may be back-scaled in a similar way.

Back-scaled output 1626 may be determined using the following equation.

α_{back-scaled}=α_{scaled}·yScale+yMean,

where α_{scaled} is the value in output layer 1606, α_{backscaled} is back-scaled output 1626 and yscale and yMean are set forth in Table 23. yscale and yMean are analogous to xScale and xMean. yMax is analogous to xMax. yMax is also set forth, for model 1600, in Table 23.

TABLE 23

| Time-point | yMax | yMean | yScale |
|---|---|---|---|
| 1 | 0.5 | 11.396 | 25.2079 |
| 2 | 0.5 | 21.0237 | 33.9525 |
| 3 | 0.5 | 40.0661 | 46.1322 |
| 4 | 0.5 | 54.0028 | 64.0056 |
| 5 | 0.5 | 61.7921 | 76.4158 |
| 6 | 0.5 | 74.5096 | 91.0192 |
| 7 | 0.8 | 84.2147 | 64.0184 |
| 8 | 0.85 | 90.4588 | 64.0691 |

Weights 1616 (a 10×24 element array), biases 1618 (a 10×1 vector), weights 1620 (a 1×10 vector) and biasₒᵤₜₚᵤₜ 1622 (a scalar) for each of the 8 time-points are set forth in Appendix A, below.

Output layer 1606 value α_{scaled} for each of the time-points may be calculated as follows: Illustrative transfer function f is the hyperbolic tangent and is applied at each of the neurons in layers 1604 and 1606. The hyperbolic tangent is defined as:

$$f ( n ) = \frac{{e}^{n} - {e}^{- n}}{{e}^{n} + {e}^{- n}} .$$

The value of each of the neurons in hidden layer 1604 is αⱼ, where j=1 to 10. The values αⱼ are calculated as follows:

$${a}_{j} = f ( {\sum }_{i = 1 to 24} {W}_{ji} {p}_{{scaled }_{i}} + {b}_{j} ) ,$$

where Wⱼᵢ are weights 1616, _{scaled}_{ᵢ} are scaled inputs 1614, bⱼ are biases 1618 and f is defined by f(n) above.

The value of the neuron in output layer 1606 (α_{scaled}) is given by:

$${a}_{scaled} = f ( {\sum }_{j = 1 to 10} {W}_{j} {a}_{j} + {b}_{2} ) ,$$

where Wⱼ are weights 1620, αⱼ are defined above, b₂ is biasₒᵤₜₚᵤₜ 1622 and f is defined by f(n) above.

Model 1600 may be executed in MATLAB by loading the aforementioned scalar, vector and 2-D array variables into MATLAB variables and performing the calculations defined by the foregoing equations. It will be understood that model 1600 may be executed using any suitable numerical analysis platform. The model may be executed manually.

Model 1600 may be validated using Leave-One-Out Cross-Validation (“LOOCV”) in which a sample of the training data set is predicted using the remaining portion of the training data set. One batch of tablets was removed from model 1600, which was retrained without the batch. Dissolution of the batch was then predicted using model 1600. The root-mean-square error of prediction (“RMSEP”) was then calculated by comparing the predicted to the actual dissolution profile at the specification time points for profiles in which the actual and/or predicted profiles met the dissolution acceptance criteria. This procedure was repeated until all tablet batches had in turn been left out and predicted. The root-mean-square error of cross-validation (RMSECV) is the average of all the individual RMSEPs.

The RMSECV for model 1600 for the formulations is 2.9% when operating within acceptance criteria ranges. The ratio of hypromelloses can be determined by targeting the mid-points at the 6 and 12 hours dissolution time points. With acceptance criteria ranges of 22% and 30% at 6 and 12 hours, respectively, a RMSECV of 2.9% for model 1600 compares favorably with the acceptance criteria ranges.

Model 1600 is a tool that may be used to increase batch performance, as measured by in vitro dissolution of tablets. As a result, the model is considered verified if the tablets meet the in vitro dissolution acceptance criteria.

Twenty-four batches of tablets in total, 6 batches of each strength, were manufactured, at 2 commercial sites, using hypromellose 100 cp to 4000 cp ratios determined using the ANN. Details of the manufacture are provided above.

All batches of 200, 300 and 400 mg strength tablets met the dissolution acceptance criteria. Four of the 6 batches of 50 mg strength tablets met the dissolution acceptance criteria. Two 50 mg tablet batches did not meet the acceptance criteria, and these batches were made from the same lots of hypromellose 100 cp and 4000 cp at each of the two commercial manufacturing sites. Since the model has been trained based on hypromellose commercial availability, there are hypromellose compositions that are under-represented in the training. For example, the hydroxypropoxy content (10%) of the 4000 cp hypromellose of the failed batches is a content level that is not well-represented in the training tablets.

The development of model 1600 has demonstrated that model refinement, e.g., based on increasing the number of the hypromellose lots and tablet batches, the variety of formulation strengths, and perhaps other variables, may increase model robustness.

Data corresponding to the tablets upon which model 1600 was trained are set forth below in Table 24.

TABLE 24

| | HPMC | | | | | | | | | |
|---|---|---|---|---|---|---|---|---|---|---|
| Tablet | 100 cp | 4000 cp | Dissolution (%) | | | | | | | |
| Strength | (%) | (%) | 1 hour | 2 hour | 4 hour | 6 hour | 8 hour | 12 hour | 16 hour | 20 hour |
| 50 | 24 | 6 | 13.2 | 23.3 | 43.5 | 56.5 | 62.5 | 75.9 | 90.6 | 100.7 |
| 50 | 24 | 6 | 12.9 | 22.8 | 43.4 | 56.8 | 63.4 | 76.9 | 90.6 | 99.9 |
| 50 | 24 | 6 | 13.9 | 24.1 | 44.3 | 57.3 | 63.7 | 77.1 | 90.2 | 99.7 |
| 50 | 24 | 6 | 13.5 | 23.8 | 45.6 | 59.8 | 67.5 | 85.6 | 101.6 | 107.2 |
| 50 | 24 | 6 | 13.7 | 25.1 | 48.1 | 63.3 | 72.2 | 95.2 | 105.4 | 106.2 |
| 50 | 24 | 6 | 13.4 | 24.0 | 45.9 | 60.8 | 68.7 | 88.7 | 102.6 | 104.9 |
| 50 | 24 | 6 | 19.5 | 32.1 | 57.2 | 73.5 | 87.0 | 103.0 | 104.1 | 103.9 |
| 50 | 24 | 6 | 20.2 | 31.2 | 53.4 | 68.4 | 77.9 | 100.3 | 106.1 | 107.1 |
| 50 | 24 | 6 | 16.3 | 26.8 | 48.1 | 61.9 | 68.5 | 85.8 | 98.5 | 102.3 |
| 50 | 24 | 6 | 13.2 | 24.4 | 46.1 | 60.5 | 67.7 | 85.0 | 100.6 | 105.9 |
| 50 | 22 | 8 | 12.8 | 23.1 | 42.8 | 56.7 | 62.9 | 76.2 | 90.0 | 100.8 |
| 50 | 23 | 7 | 15.4 | 25.3 | 44.0 | 57.0 | 62.8 | 72.0 | 82.8 | 93.1 |
| 50 | 23 | 7 | 12.9 | 23.4 | 43.8 | 57.9 | 64.5 | 79.2 | 93.8 | 102.8 |
| 50 | 23 | 7 | 13.5 | 23.9 | 44.0 | 57.9 | 64.6 | 79.8 | 95.1 | 104.6 |
| 50 | 23 | 7 | 13.5 | 24.0 | 45.2 | 59.4 | 66.4 | 83.3 | 97.9 | 102.7 |
| 50 | 23 | 7 | 13.9 | 24.9 | 44.8 | 59.8 | 65.8 | 81.0 | 95.5 | 103.3 |
| 50 | 23 | 7 | 13.1 | 23.7 | 42.9 | 56.5 | 62.4 | 75.9 | 89.6 | 99.9 |
| 50 | 23 | 7 | 15.0 | 24.6 | 43.3 | 56.8 | 62.9 | 74.6 | 87.4 | 99.1 |
| 50 | 23 | 7 | 13.0 | 23.1 | 42.8 | 57.1 | 63.6 | 78.2 | 93.7 | 102.8 |
| 50 | 23 | 7 | 12.2 | 21.4 | 39.5 | 53.2 | 59.0 | 71.5 | 86.3 | 99.0 |
| 50 | 23 | 7 | 13.9 | 23.4 | 41.8 | 55.0 | 60.5 | 71.2 | 83.7 | 96.1 |
| 50 | 23 | 7 | 12.9 | 22.4 | 40.2 | 53.4 | 58.9 | 70.0 | 83.2 | 96.1 |
| 50 | 23 | 7 | 13.1 | 22.9 | 40.7 | 54.2 | 59.7 | 70.9 | 83.5 | 95.6 |
| 50 | 23 | 7 | 13.2 | 23.1 | 42.2 | 56.2 | 62.2 | 75.1 | 89.3 | 101.0 |
| 50 | 23 | 7 | 13.5 | 23.4 | 42.6 | 57.0 | 63.4 | 77.2 | 91.9 | 103.3 |
| 50 | 23 | 7 | 15.4 | 25.7 | 44.8 | 58.2 | 63.9 | 75.3 | 88.6 | 100.5 |
| 50 | 23 | 7 | 12.8 | 23.0 | 42.0 | 55.6 | 61.4 | 73.4 | 87.2 | 99.1 |
| 50 | 23 | 7 | 12.5 | 22.4 | 40.9 | 54.2 | 59.8 | 72.2 | 85.7 | 97.1 |
| 50 | 23 | 7 | 13.1 | 23.1 | 41.8 | 55.1 | 61.1 | 73.1 | 87.0 | 98.5 |
| 50 | 23 | 7 | 13.5 | 23.4 | 41.6 | 54.6 | 60.0 | 70.6 | 82.8 | 95.1 |
| 50 | 23 | 7 | 14.0 | 24.0 | 42.6 | 55.8 | 61.6 | 75.0 | 89.6 | 100.6 |
| 50 | 23 | 7 | 13.0 | 22.8 | 41.2 | 54.2 | 59.4 | 70.0 | 82.0 | 93.7 |
| 50 | 23 | 7 | 12.9 | 22.7 | 41.1 | 54.4 | 60.0 | 71.1 | 84.8 | 96.8 |
| 50 | 23 | 7 | 12.5 | 21.2 | 38.3 | 51.3 | 56.8 | 69.2 | 83.1 | 95.8 |
| 50 | 23 | 7 | 12.7 | 21.6 | 38.9 | 52.2 | 57.8 | 68.3 | 82.5 | 95.5 |
| 50 | 23 | 7 | 13.7 | 23.9 | 43.3 | 57.2 | 63.2 | 76.1 | 91.0 | 101.7 |
| 200 | 23 | 7 | 10.6 | 21.1 | 42.6 | 59.0 | 68.1 | 82.2 | 92.6 | 97.8 |
| 200 | 23 | 7 | 10.1 | 20.3 | 41.3 | 57.3 | 66.1 | 79.7 | 90.6 | 96.6 |
| 200 | 23 | 7 | 9.5 | 19.4 | 39.7 | 55.2 | 63.2 | 75.5 | 88.2 | 98.0 |
| 200 | 23 | 7 | 10.8 | 21.6 | 44.6 | 63.2 | 76.5 | 97.4 | 101.3 | 101.4 |
| 200 | 23 | 7 | 13.3 | 23.4 | 45.1 | 62.0 | 71.7 | 89.5 | 101.6 | 104.6 |
| 200 | 23 | 7 | 10.2 | 18.8 | 36.3 | 49.5 | 56.2 | 66.0 | 76.7 | 87.4 |
| 200 | 23 | 7 | 9.6 | 19.6 | 39.5 | 54.4 | 61.8 | 72.8 | 83.7 | 92.4 |
| 200 | 23 | 7 | 9.8 | 19.7 | 39.7 | 55.1 | 63.1 | 75.6 | 87.9 | 96.0 |
| 200 | 23 | 7 | 9.5 | 19.5 | 39.4 | 54.9 | 63.2 | 77.0 | 89.8 | 95.9 |
| 200 | 23 | 7 | 9.6 | 19.7 | 39.9 | 55.2 | 63.7 | 77.3 | 89.8 | 97.9 |
| 200 | 23 | 7 | 8.9 | 18.1 | 36.6 | 50.5 | 58.0 | 69.1 | 80.8 | 91.0 |
| 200 | 23 | 7 | 8.4 | 16.4 | 31.9 | 43.4 | 49.4 | 58.2 | 65.4 | 73.2 |
| 200 | 23 | 7 | 10.0 | 20.8 | 42.7 | 60.0 | 69.6 | 85.1 | 95.7 | 100.2 |
| 200 | 23 | 7 | 10.0 | 20.1 | 40.9 | 56.5 | 64.5 | 76.9 | 87.5 | 93.5 |
| 200 | 23 | 7 | 9.7 | 20.2 | 41.5 | 57.0 | 64.7 | 76.8 | 87.9 | 94.6 |
| 200 | 23 | 7 | 10.4 | 21.2 | 43.1 | 59.4 | 67.7 | 82.1 | 94.0 | 99.5 |
| 200 | 23 | 7 | 10.0 | 20.4 | 41.3 | 57.0 | 64.8 | 77.4 | 89.7 | 96.6 |
| 200 | 23 | 7 | 10.1 | 20.6 | 42.0 | 57.6 | 65.7 | 78.6 | 89.9 | 95.2 |
| 200 | 23 | 7 | 9.5 | 19.2 | 39.4 | 54.9 | 62.9 | 76.1 | 88.3 | 95.6 |
| 200 | 23 | 7 | 10.5 | 21.0 | 42.8 | 58.8 | 67.0 | 80.5 | 92.2 | 98.7 |
| 200 | 23 | 7 | 11 | 22 | 43 | 60 | 68 | 83 | 95 | 98 |
| 200 | 23 | 7 | 12 | 23 | 44 | 61 | 72 | 93 | 99 | 100 |
| 200 | 23 | 7 | 9 | 18 | 35 | 48 | 55 | 63 | 71 | 80 |
| 200 | 23 | 7 | 9.8 | 19.8 | 39.7 | 54.2 | 61.9 | 73.7 | 87.0 | 96.5 |
| 200 | 23 | 7 | 9.6 | 19.0 | 38.1 | 52.5 | 59.9 | 71.4 | 84.7 | 95.6 |
| 200 | 23 | 7 | 9.0 | 18.6 | 38.5 | 53.5 | 61.3 | 73.7 | 85.8 | 95.0 |
| 200 | 23 | 7 | 10.4 | 20.3 | 40.2 | 55.0 | 62.2 | 73.7 | 87.0 | 96.5 |
| 200 | 23 | 7 | 9.1 | 18.1 | 36.0 | 49.6 | 56.3 | 65.9 | 79.7 | 93.5 |
| 200 | 23 | 7 | 10.0 | 20.6 | 41.5 | 57.1 | 65.6 | 79.5 | 91.4 | 97.9 |
| 200 | 23 | 7 | 9.7 | 19.7 | 39.8 | 55.5 | 63.8 | 77.3 | 89.4 | 97.0 |
| 200 | 23 | 7 | 9.7 | 19.7 | 40.0 | 55.9 | 64.3 | 77.8 | 90.3 | 98.0 |
| 200 | 23 | 7 | 11.0 | 21.2 | 41.8 | 58.0 | 66.8 | 81.0 | 91.8 | 97.3 |
| 200 | 23 | 7 | 10.0 | 21.3 | 44.3 | 62.0 | 74.3 | 93.5 | 98.8 | 101.1 |
| 200 | 23 | 7 | 10.5 | 21.6 | 44.7 | 62.5 | 73.8 | 92.1 | 98.8 | 101.5 |
| 200 | 23 | 7 | 9.3 | 19.1 | 38.6 | 53.7 | 61.1 | 72.6 | 86.2 | 96.3 |
| 300 | 25 | 5 | 9.4 | 19.8 | 41.0 | 56.8 | 65.4 | 80.6 | 91.7 | 95.4 |
| 300 | 25 | 5 | 11.3 | 22.8 | 46.1 | 64.5 | 78.5 | 97.7 | 101.1 | 101.7 |
| 300 | 25 | 5 | 11.5 | 22.6 | 44.4 | 61.6 | 74.2 | 93.7 | 99.8 | 102.0 |
| 300 | 25 | 5 | 10.5 | 19.7 | 37.4 | 50.4 | 56.9 | 65.7 | 74.8 | 83.9 |
| 300 | 25 | 5 | 9.4 | 20.1 | 41.8 | 58.0 | 66.2 | 78.5 | 88.2 | 92.8 |
| 300 | 25 | 5 | 9.0 | 19.0 | 39.4 | 55.0 | 63.6 | 77.7 | 89.3 | 94.5 |
| 300 | 25 | 5 | 9.0 | 18.8 | 38.4 | 52.9 | 60.4 | 71.8 | 83.4 | 91.7 |
| 300 | 25 | 5 | 8.7 | 18.4 | 37.9 | 53.1 | 61.3 | 74.8 | 88.3 | 95.5 |
| 300 | 25 | 5 | 8.3 | 18.0 | 37.7 | 53.1 | 60.9 | 75.6 | 90.6 | 95.5 |
| 300 | 25 | 5 | 9.1 | 18.8 | 38.2 | 52.8 | 59.4 | 70.6 | 83.9 | 92.4 |
| 300 | 25 | 5 | 9.4 | 18.9 | 37.7 | 51.7 | 58.0 | 66.9 | 77.9 | 88.5 |
| 300 | 25 | 5 | 9.8 | 20.7 | 42.2 | 58.8 | 67.9 | 82.9 | 94.6 | 99.9 |
| 300 | 25 | 5 | 9.0 | 19.0 | 38.6 | 53.4 | 61.0 | 72.9 | 84.8 | 92.4 |
| 300 | 25 | 5 | 9.6 | 19.9 | 40.5 | 58.5 | 67.3 | 81.0 | 91.1 | 95.8 |
| 300 | 25 | 5 | 8.9 | 18.8 | 38.4 | 53.3 | 60.4 | 71.6 | 84.1 | 91.1 |
| 400 | 27 | 3 | 9.5 | 20.2 | 41.4 | 57.0 | 66.6 | 82.1 | 91.7 | 95.2 |
| 400 | 27 | 3 | 10.4 | 22.4 | 46.0 | 63.2 | 73.2 | 87.6 | 93.4 | 95.8 |
| 400 | 27 | 3 | 10.3 | 22.3 | 45.8 | 62.7 | 72.7 | 87.0 | 93.3 | 95.8 |
| 400 | 27 | 3 | 11.1 | 23.4 | 48.1 | 65.8 | 79.7 | 97.5 | 101.0 | 101.7 |
| 400 | 27 | 3 | 11.2 | 23.0 | 46.6 | 64.1 | 77.5 | 94.9 | 98.0 | 99.2 |
| 400 | 27 | 3 | 10.3 | 20.2 | 39.5 | 53.9 | 61.4 | 74.0 | 85.9 | 94.5 |
| 400 | 27 | 3 | 11.2 | 23.6 | 48.5 | 66.6 | 81.1 | 97.7 | 100.1 | 100.8 |
| 400 | 27 | 3 | 11.7 | 24.2 | 49.2 | 67.2 | 81.0 | 97.5 | 100.1 | 100.7 |
| 400 | 22 | 8 | 8.7 | 17.3 | 34.6 | 47.4 | 55.1 | 66.3 | 76.1 | 84.7 |
| 400 | 27 | 3 | 9.3 | 20.2 | 41.5 | 57.3 | 67.0 | 82.4 | 92.2 | 95.2 |
| 400 | 27 | 3 | 9.2 | 20.0 | 41.1 | 56.2 | 64.3 | 77.0 | 88.1 | 94.2 |
| 400 | 27 | 3 | 9.8 | 21.6 | 44.6 | 61.0 | 70.9 | 86.5 | 94.5 | 96.5 |
| 400 | 27 | 3 | 9.8 | 21.2 | 43.9 | 60.4 | 71.5 | 88.2 | 95.7 | 97.5 |
| 400 | 27 | 3 | 9.1 | 19.9 | 40.9 | 56.1 | 64.5 | 77.9 | 90.0 | 96.1 |
| 400 | 27 | 3 | 9.5 | 21.0 | 43.7 | 60.1 | 70.6 | 87.1 | 94.0 | 95.9 |
| 400 | 27 | 3 | 9.6 | 21.0 | 43.3 | 59.4 | 68.8 | 87.3 | 96.5 | 98.4 |
| 400 | 27 | 3 | 9.3 | 19.7 | 40.4 | 55.3 | 62.9 | 76.3 | 89.5 | 94.9 |
| 400 | 27 | 3 | 9.4 | 20.6 | 42.2 | 57.9 | 65.8 | 79.7 | 90.6 | 94.5 |
| 400 | 27 | 3 | 9.5 | 20.1 | 41.0 | 56.6 | 65.6 | 81.8 | 91.7 | 94.3 |
| 400 | 27 | 3 | 10.0 | 22.1 | 46.6 | 63.5 | 76.6 | 91.0 | 94.3 | 96.0 |
| 400 | 27 | 3 | 10.0 | 21.7 | 44.5 | 60.9 | 70.1 | 85.6 | 94.1 | 96.3 |
| 50 | 29 | 1 | 20.0 | 33.0 | 57.0 | 73.0 | 92.0 | 102.0 | 102.0 | 103.0 |
| 50 | 29 | 1 | 18.0 | 29.0 | 47.0 | 58.0 | 62.0 | 72.0 | 87.0 | 99.0 |
| 50 | 29 | 1 | 24.0 | 38.0 | 63.0 | 82.0 | 100.0 | 102.0 | 102.0 | 103.0 |
| 50 | 29 | 1 | 17.0 | 27.0 | 45.0 | 57.0 | 61.0 | 70.0 | 80.0 | 94.0 |
| 50 | 29 | 1 | 21.0 | 34.0 | 57.0 | 72.0 | 85.0 | 104.0 | 105.0 | 105.0 |
| 200 | 29 | 1 | 13.0 | 26.0 | 52.0 | 71.0 | 84.0 | 101.0 | 102.0 | 102.0 |
| 200 | 29 | 1 | 12.0 | 25.0 | 49.0 | 66.0 | 74.0 | 92.0 | 99.0 | 102.0 |
| 200 | 29 | 1 | 12.0 | 20.0 | 36.0 | 48.0 | 53.0 | 60.0 | 69.0 | 84.0 |
| 200 | 29 | 1 | 13.0 | 26.0 | 53.0 | 72.0 | 85.0 | 102.0 | 103.0 | 103.0 |
| 200 | 29 | 1 | 13.0 | 24.0 | 49.0 | 64.0 | 70.0 | 85.0 | 95.0 | 98.0 |
| 300 | 29 | 1 | 12.0 | 24.0 | 48.0 | 66.0 | 78.0 | 98.0 | 101.0 | 101.0 |
| 300 | 29 | 1 | 12.0 | 23.0 | 46.0 | 63.0 | 72.0 | 91.0 | 97.0 | 99.0 |
| 300 | 29 | 1 | 8.0 | 17.0 | 35.0 | 47.0 | 53.0 | 61.0 | 77.0 | 88.0 |
| 300 | 29 | 1 | 12.0 | 24.0 | 48.0 | 67.0 | 79.0 | 99.0 | 101.0 | 102.0 |
| 300 | 29 | 1 | 12.0 | 22.0 | 42.0 | 56.0 | 62.0 | 81.0 | 99.0 | 100.0 |
| 400 | 29 | 1 | 12.0 | 24.0 | 49.0 | 66.0 | 79.0 | 94.0 | 97.0 | 99.0 |
| 400 | 29 | 1 | 12.0 | 25.0 | 50.0 | 67.0 | 77.0 | 90.0 | 95.0 | 97.0 |
| 400 | 29 | 1 | 10.0 | 20.0 | 39.0 | 52.0 | 58.0 | 66.0 | 77.0 | 87.0 |
| 400 | 29 | 1 | 12.0 | 25.0 | 50.0 | 68.0 | 79.0 | 93.0 | 96.0 | 97.0 |
| 400 | 29 | 1 | 11.0 | 22.0 | 44.0 | 59.0 | 67.0 | 85.0 | 97.0 | 99.0 |
| 50 | 22 | 8 | 18.0 | 30.0 | 54.0 | 71.0 | 86.0 | 103.0 | 103.0 | 103.0 |
| 50 | 22 | 8 | 22.0 | 32.0 | 53.0 | 67.0 | 75.0 | 93.0 | 103.0 | 103.0 |
| 50 | 22 | 8 | 16.0 | 25.0 | 44.0 | 57.0 | 62.0 | 74.0 | 87.0 | 98.0 |
| 50 | 22 | 8 | 18.0 | 26.0 | 42.0 | 53.0 | 56.0 | 61.0 | 67.0 | 72.0 |
| 50 | 22 | 8 | 16.0 | 25.0 | 41.0 | 51.0 | 56.0 | 63.0 | 70.0 | 76.0 |
| 50 | 22 | 8 | 16.0 | 26.0 | 43.0 | 55.0 | 60.0 | 71.0 | 82.0 | 95.0 |
| 50 | 22 | 8 | 16.0 | 21.0 | 42.0 | 54.0 | 59.0 | 65.0 | 73.0 | 81.0 |
| 200 | 22 | 8 | 11.0 | 21.0 | 43.0 | 60.0 | 73.0 | 94.0 | 102.0 | 103.0 |
| 200 | 22 | 8 | 10.0 | 19.0 | 38.0 | 53.0 | 62.0 | 76.0 | 92.0 | 101.0 |
| 200 | 22 | 8 | 9.0 | 18.0 | 35.0 | 49.0 | 56.0 | 66.0 | 76.0 | 85.0 |
| 200 | 22 | 8 | 9.0 | 16.0 | 28.0 | 37.0 | 41.0 | 47.0 | 53.0 | 58.0 |
| 200 | 22 | 8 | 10.0 | 17.0 | 30.0 | 40.0 | 44.0 | 50.0 | 56.0 | 60.0 |
| 200 | 22 | 8 | 9.0 | 16.0 | 30.0 | 41.0 | 46.0 | 54.0 | 60.0 | 68.0 |
| 300 | 22 | 8 | 9.0 | 19.0 | 39.0 | 50.0 | 67.0 | 88.0 | 100.0 | 102.0 |
| 300 | 22 | 8 | 9.0 | 17.0 | 34.0 | 48.0 | 56.0 | 69.0 | 86.0 | 97.0 |
| 300 | 22 | 8 | 9.0 | 17.0 | 34.0 | 47.0 | 53.0 | 62.0 | 69.0 | 75.0 |
| 300 | 22 | 8 | 8.0 | 13.0 | 24.0 | 31.0 | 35.0 | 40.0 | 45.0 | 49.0 |
| 300 | 22 | 8 | 8.0 | 15.0 | 28.0 | 39.0 | 44.0 | 52.0 | 58.0 | 64.0 |
| 400 | 22 | 8 | 9.0 | 19.0 | 38.0 | 54.0 | 67.0 | 86.0 | 97.0 | 99.0 |
| 400 | 22 | 8 | 8.0 | 17.0 | 34.0 | 48.0 | 58.0 | 75.0 | 91.0 | 96.0 |
| 400 | 22 | 8 | 9.0 | 18.0 | 37.0 | 51.0 | 59.0 | 73.0 | 88.0 | 92.0 |
| 400 | 22 | 8 | 8.0 | 15.0 | 28.0 | 38.0 | 43.0 | 50.0 | 56.0 | 64.0 |
| 400 | 22 | 8 | 8.0 | 15.0 | 29.0 | 40.0 | 46.0 | 54.0 | 61.0 | 68.0 |
| 400 | 22 | 8 | 9.0 | 16.0 | 32.0 | 43.0 | 50.0 | 58.0 | 67.0 | 80.0 |
| 50 | 15 | 15 | 16.0 | 26.0 | 46.0 | 60.0 | 68.0 | 88.0 | 99.0 | 100.0 |
| 50 | 15 | 15 | 18.0 | 27.0 | 43.0 | 57.0 | 60.0 | 69.0 | 78.0 | 87.0 |
| 50 | 15 | 15 | 19.0 | 27.0 | 43.0 | 55.0 | 60.0 | 68.0 | 75.0 | 82.0 |
| 50 | 15 | 15 | 16.0 | 23.0 | 35.0 | 43.0 | 48.0 | 53.0 | 58.0 | 62.0 |
| 50 | 15 | 15 | 16.0 | 23.0 | 36.0 | 44.0 | 49.0 | 54.0 | 58.0 | 62.0 |
| 50 | 15 | 15 | 14.0 | 21.4 | 34.0 | 42.8 | 47.0 | 53.0 | 57.6 | 61.0 |
| 200 | 15 | 15 | 9.0 | 17.0 | 34.0 | 48.0 | 59.0 | 76.0 | 93.0 | 103.0 |
| 200 | 15 | 15 | 8.0 | 14.0 | 27.0 | 36.0 | 41.0 | 49.0 | 55.0 | 61.0 |
| 200 | 15 | 15 | 8.0 | 15.0 | 28.0 | 38.0 | 44.0 | 53.0 | 60.0 | 65.0 |
| 200 | 15 | 15 | 8.0 | 13.0 | 22.0 | 28.0 | 32.0 | 37.0 | 41.0 | 45.0 |
| 200 | 15 | 15 | 8.0 | 12.0 | 21.0 | 28.0 | 31.0 | 36.0 | 40.0 | 44.0 |
| 200 | 15 | 15 | 7.0 | 12.0 | 22.0 | 29.0 | 32.0 | 37.0 | 42.0 | 46.0 |
| 300 | 15 | 15 | 8.0 | 15.0 | 29.0 | 42.0 | 52.0 | 68.0 | 84.0 | 97.0 |
| 300 | 15 | 15 | 7.0 | 13.0 | 25.0 | 35.0 | 41.0 | 50.0 | 58.0 | 65.0 |
| 300 | 15 | 15 | 7.0 | 13.0 | 24.0 | 32.0 | 37.0 | 44.0 | 50.0 | 55.0 |
| 300 | 15 | 15 | 7.0 | 11.0 | 19.0 | 25.0 | 28.0 | 32.0 | 36.0 | 39.0 |
| 300 | 15 | 15 | 7.0 | 12.0 | 20.0 | 26.0 | 28.0 | 33.0 | 36.0 | 39.0 |
| 300 | 15 | 15 | 6.0 | 10.0 | 17.0 | 22.0 | 25.0 | 29.0 | 33.0 | 36.0 |
| 400 | 15 | 15 | 7.0 | 14.0 | 30.0 | 42.0 | 53.0 | 70.0 | 83.0 | 92.0 |
| 400 | 15 | 15 | 7.0 | 13.0 | 25.0 | 35.0 | 42.0 | 51.0 | 60.0 | 69.0 |
| 400 | 15 | 15 | 7.0 | 13.0 | 26.0 | 37.0 | 43.0 | 53.0 | 60.0 | 67.0 |
| 400 | 15 | 15 | 7.0 | 11.0 | 19.0 | 25.0 | 28.0 | 32.0 | 36.0 | 40.0 |
| 400 | 15 | 15 | 6.0 | 11.0 | 20.0 | 26.0 | 30.0 | 35.0 | 40.0 | 43.0 |
| 400 | 15 | 15 | 6.0 | 11.0 | 22.0 | 30.0 | 34.0 | 41.0 | 46.0 | 51.0 |
| 50 | 15 | 15 | 20.3 | 29.0 | 44.6 | 55.9 | 60.5 | 68.2 | 76.4 | 85.2 |
| 50 | 15 | 15 | 17.6 | 25.7 | 39.8 | 50.2 | 54.5 | 60.7 | 65.8 | 70.0 |

Table 25 lists characteristics of the 100 cp hypromellose lots used to train model 1600.

TABLE 25

| HP | MeO | CoA_100 | CoA | | | Particle | Particle |
|---|---|---|---|---|---|---|---|
| (Weight %) | (Weight %) | mesh | Viscosity | Mw | Mn | D50 | Span |
| 10.4 | 27.4 | 96.5 | 103 | 131000 | 46800 | 82.1 | 2.21 |
| 10.8 | 27.6 | 91.2 | 96 | 131000 | 43900 | 64.0 | 2.00 |
| 10.5 | 28.2 | 93.0 | 102 | 124000 | 47000 | 104.1 | 2.42 |
| 10.5 | 29.0 | 91.8 | 105 | 131300 | 41300 | 77.7 | 2.55 |
| 10.6 | 29.0 | 92.9 | 96 | 123000 | 43800 | 83.4 | 2.29 |
| 10.7 | 28.7 | 94.8 | 97 | 133300 | 38500 | 66.3 | 2.66 |
| 10.8 | 27.7 | 95.0 | 112 | 136000 | 45600 | 83.2 | 2.63 |
| 10.6 | 29.2 | 91.2 | 102 | 133000 | 44600 | 78.6 | 2.67 |
| 11.8 | 26.4 | 93.7 | 103 | 138000 | 44800 | 72.3 | 2.69 |
| 13.4 | 29.1 | 94.9 | 103 | 147000 | 51400 | 77.7 | 2.75 |
| 9.8 | 28.5 | 94.6 | 101 | 130000 | 42800 | 69.7 | 2.39 |
| 10.6 | 28.4 | 93.0 | 110 | 130000 | 43100 | 68.4 | 2.59 |
| 10.7 | 27.9 | 92.8 | 100 | 128000 | 39500 | 71.1 | 2.96 |
| 9.9 | 28.9 | 96.2 | 108 | 136000 | 56500 | 63.3 | 2.87 |
| 9.9 | 28.8 | 92.8 | 112 | 142000 | 42100 | 63.1 | 2.46 |
| 11.2 | 27.9 | 95.8 | 100 | 125000 | 43000 | 68.9 | 2.76 |
| 10.6 | 27.5 | 96.6 | 103 | 134000 | 46500 | 75.7 | 2.56 |
| 10.6 | 28.2 | 100.0 | 102 | 130000 | 55000 | 67.4 | 2.09 |
| 10.7 | 28.5 | 100.0 | 101 | 130000 | 55000 | 65.7 | 2.14 |
| 10.7 | 28.5 | 100.0 | 104 | 131000 | 56000 | 64.3 | 2.10 |

Table 26 lists characteristics of the 4000 cp hypromellose lots used to train model 1600.

TABLE 26

| HP | MeO | CoA_100 | CoA | | | Particle | Particle |
|---|---|---|---|---|---|---|---|
| (Weight %) | (Weight %) | mesh | Viscosity | Mw | Mn | D50 | Span |
| 11.3 | 28.8 | 95.0 | 5280 | 351000 | 130200 | 75.4 | 2.55 |
| 11.3 | 27.8 | 93.8 | 3684 | 327000 | 112000 | 67.9 | 2.57 |
| 11.6 | 29.0 | 90.0 | 5436 | 328000 | 101300 | 81.1 | 2.46 |
| 12.1 | 27.3 | 91.7 | 4184 | 304000 | 84000 | 75.0 | 2.93 |
| 11.9 | 28.5 | 95.0 | 5151 | 350300 | 94400 | 80.5 | 2.25 |
| 11.6 | 28.1 | 92.0 | 4782 | 331000 | 110000 | 107.6 | 2.25 |
| 11.5 | 29.0 | 93.2 | 4556 | 333000 | 140000 | 88.2 | 2.44 |
| 10.6 | 27.7 | 93.8 | 4829 | 331000 | 97000 | 81.5 | 2.57 |
| 10.8 | 28.7 | 91.6 | 5227 | 332000 | 94700 | 92.3 | 2.52 |
| 9.9 | 28.2 | 95.3 | 3962 | 313000 | 88000 | 99.6 | 2.51 |
| 10.0 | 28.6 | 93.5 | 5535 | 325000 | 105000 | 55.3 | 2.83 |
| 12.8 | 27.6 | 90.7 | 4591 | 325000 | 118000 | 88.8 | 2.60 |
| 11.4 | 29.0 | 96.6 | 5005 | 329000 | 101000 | 80.9 | 2.07 |

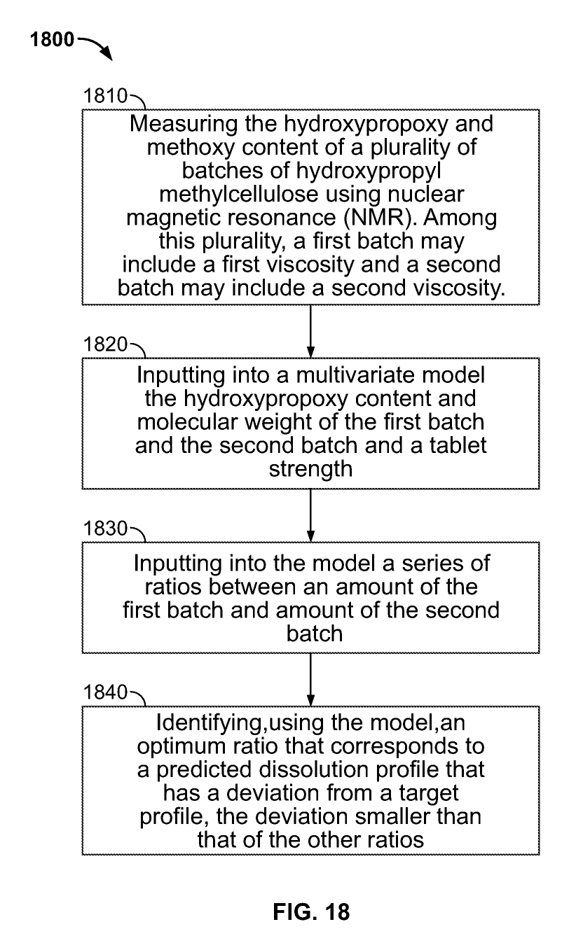

FIG. 18 shows illustrative method 1800 for formulating an extended release formulation. The method may include step 1810 of measuring the hydroxypropoxy and methoxy of a plurality of hypromellose lots using nuclear magnetic resonance (NMR). Among this plurality, a first lot may have a first viscosity and a second lot may have a second viscosity. Step 1820 shows inputting into a multivariate model the hydroxypropoxy content and molecular weight of the first lot and the second lot and a tablet strength. Step 1830 shows inputting into the model a series of ratios between an amount of the first lot and an amount of the second lot. Step 1840 shows identifying, using the model, an optimum ratio that corresponds to a predicted dissolution profile that has a deviation from a target profile, the deviation being smaller than that of the other ratios.

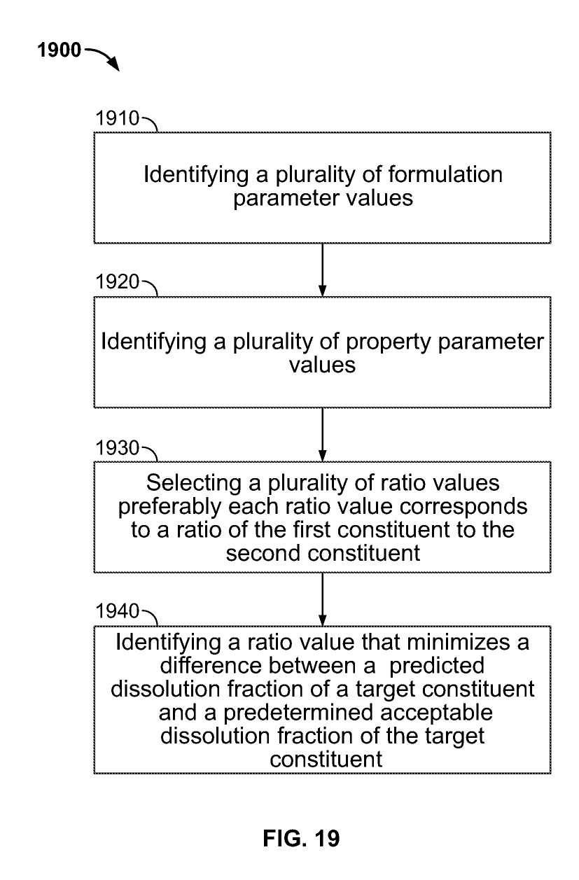

FIG. 19 shows illustrative method 1900. Method 1900 may include step 1910 of identifying a plurality of formulation parameter values. Method 1900 may include step 1920 of identifying a plurality of property parameter values. Step 1930 shows selecting a plurality of ratio values. Each ratio value may correspond to a ratio of the first constituent to the second constituent. Step 1940 shows identifying a ratio value that minimizes a difference between a predicted dissolution fraction of a target constituent and a predetermined acceptable dissolution fraction of the target constituent.

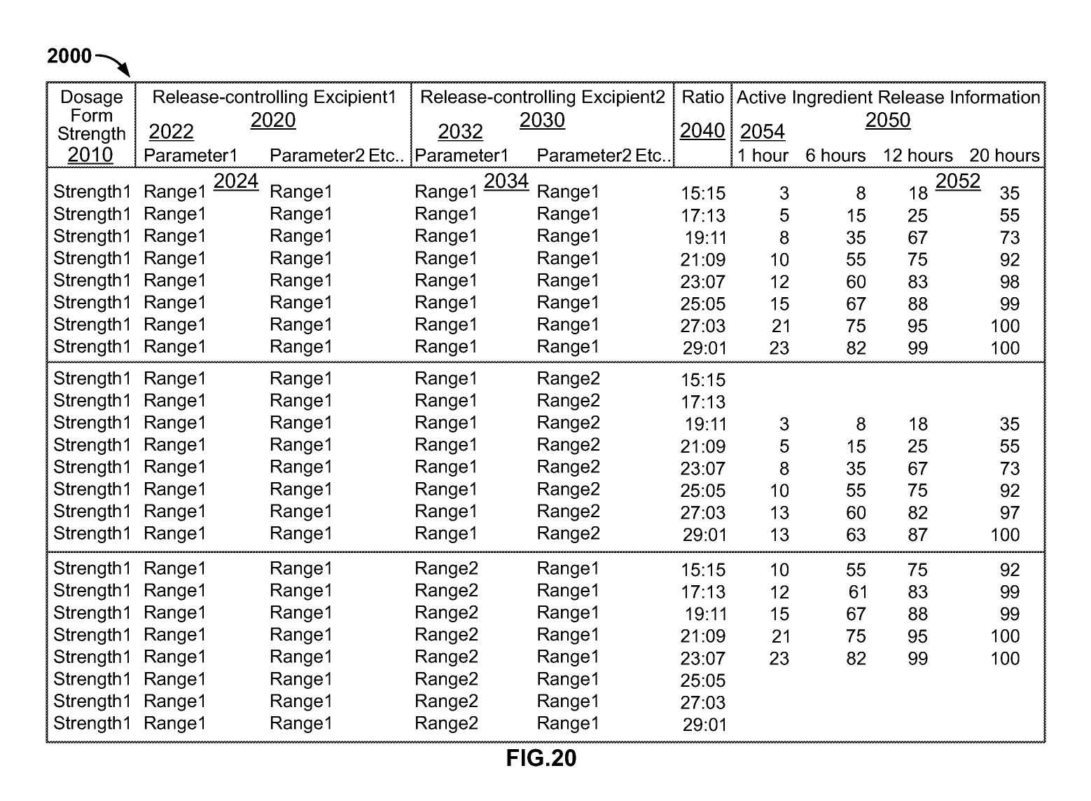

FIG. 20 shows illustrative look-up table 2000 that may be used to correlate ratio 2140 of release-controlling excipient 2120 and 2130 %-weights to active ingredient release information 2150. Information 2150 may include released percent 2152 of active ingredient at time 2154. Information 2150 may be determined in whole or in part by one or more physical or chemical parameters 2122 and 2132 of release-controlling excipients 2120 and 2130, respectively. Parameters 2122 and 2132 may be binned in ranges such as ranges 2124 and 2134, respectively. Information 2150 may be determined in whole or in part by dosage form strength 2110. Look-up table 2000 may be populated by empirically determining information 2150 for all combinations of values of strength 2110, parameters such as 2122, parameters such as 2132 and ratio 2140. In some embodiments of the invention, look-up table 2000 may be populated partially by empirically determining information 2150 for the values and partially by estimating the values. For example, some values of information 2150 may be interpolated or extrapolated based on nearby values.

In some embodiments of the invention, release-controlling excipients 2120 and 2130 may be hypromellose having nominal viscosities 100 cp and 4000 cp, respectively. In some embodiments of the invention, the active ingredient may be quetiapine. In some embodiments of the invention, parameters such as 2122 and 2132 may correspond to inputs to model 1600 (shown in FIG. 16; see, e.g., inputs 1-16 in Table 17).

### Exemplification

### EXAMPLE 1

### Determination of hydroxypropyl (HP) Content of Hypromellose (Hypromellose) by Nuclear Magnetic Resonance

According to NMR Method 2, 3.5 to about 4.5 mg of hypromellose is dissolved in a solvent, which is 99.96% D₂O. The hypromellose is heated at about 105° C. for about 30 minutes prior to dissolving in the solvent. The hypromellose is heated at about 80° C. for about 15 minutes after dissolving in the solvent. The nuclear magnetic resonance spectrometer comprises a ¹H{X} inverse detection probe. The temperature is about 353K. The pulse is about 45°. The spectrum width is about −2.5 to 13.5 ppm. The pulse repetition is about 15 seconds. The exponential line broadening is about 1.0 Hz. The spectrum is referenced to residual dimethyl sulfoxide (DMSO) peak at 2.70 ppm. The baseline of the nuclear magnetic resonance spectrum is corrected. The number of scans is selected such that the signal:noise ratio at 200 Hz for the peak at 1.2 ppm is greater than 500. The number of time domain data points is about 65,000. The number of processed data points is about 250,000. NMR spectrum is phased so that the peaks at 4.5 ppm and 1.2 ppm are symmetric.

The following regions are integrated: Region 1: 4.96-4.31, which is Area A; Region 2: 4.08-2.95, which is Area B; and Region 3: 1.47-0.92, which is Area C.

The hydroxypropoxy content (weight % HP) is calculated as:

Weight % HP={(75×MoleHP)/[162+(58×MoleHP)+(14×MoleMeO)]}×100, wherein: MoleHP=C/(3×A); MoleMeO=[B−C−(6×A)]/(3×A); and MeO is methoxy.

The following is an exemplary procedure for analysis of hydroxypropyl (HP) content of hypromellose by NMR.

According to NMR Method 2, a 3.5 to 4.5 mg sample of hypromellose is heated at about 105° C. for about 30 minutes. The 3.5 to 4.5 mg sample of hypromellose is dissolved in 99.96% D₂O. The dissolved hypromellose is heated at about 80° C. for about 10 minutes. The dissolved hypromellose is analyzed by nuclear magnetic resonance whereby (i) the nuclear magnetic resonance spectrometer comprises a ¹H{X} inverse detection probe, (ii) the temperature is about 353K, (iii) the pulse is about 45°, (iv) the spectrum width is about −3.5 to 13.5 ppm, (v) the pulse repetition is about 15 seconds, (vi) the exponential line broadening is about 1.0 Hz, (vii) the number of scans is selected such that the signal:noise ratio at 200 Hz for the peak at 1.2 ppm is greater than 500, (viii) the number of time domain data points is about 65,000, and (ix) the number of processed data points is about 250,000.

The nuclear magnetic resonance spectrum is phased so that the peaks at 4.5 ppm and 1.2 ppm are symmetric. The spectrum is referenced to residual DMSO peak at 2.70 ppm. The baseline of the nuclear magnetic resonance spectrum is corrected.

The following regions are integrated: Region 1: 4.96-4.31, which is Area A; Region 2: 4.31-4.08; Region 3: 4.08-2.95, which is Area B; Region 4: 2.95-2.45; and Region 5: 1.47-0.92, which is Area C.

The hydroxypropoxy content (weight % HP) is calculated as: Weight % HP={(75×MoleHP)/[162+(58×MoleHP)+(14×MoleMeO)]}×100, wherein (i) MoleHP=C/(3×A) and (ii) MoleMeO=[B−C−(6×A)]/(3×A).

### EXAMPLE 2

### Formulation of 50 mg Tablet

The following process was used to manufacture extended release formulations of quetiapine fumarate set forth in Table 1.

1) Mixing quetiapine fumarate, lactose, microcrystalline cellulose, Hypromellose 2208 (USP), and sodium citrate (e.g., in a high shear granulator) until content uniformity is achieved (e.g., 600 L Fielder for about 10 minutes);

2) Charging purified water (e.g., 37% by weight of the tablet) onto the powder in the granulator (e.g., spray nozzle) 5-6 minutes to form a granulate;

3) Drying the granulate in a fluid bed dryer (e.g., to a moisture content of < or equal to 3% loss on drying);

4) Reducing the particle of the granulate to achieve a suitable flow for compression (e.g., Carr index that does not exceed 30 (e.g., 20) using, e.g., 0.05 to 0.109 inch mill screen; and

5) Blending the granulate with magnesium stearate for a time sufficient to prevent substantial tablet punch filming (e.g., 3 minutes in a V blender; ⅔ full).

The resulting formulation of step 5 is compressed to form a tablet having a hardness of greater than 16 kiloponds (particularly about 28 kp) and a friability of less than 1%.

The tablets may further be coated by mixing all the coating ingredients in water until dissolved and spray the resulting mixture spray onto the tablet (for example in perforated pan coater) until a uniform coat is achieved (e.g., a target of 2.5% percent by weight).

### EXAMPLE 3

### Formulation of 150 mg Tablet

The procedure described in Example 2 was used to manufacture tablets of the composition shown in Table 2.

### EXAMPLE 4

### Formulation of 200 mg Tablet

The procedure described in Example 2 was used to manufacture tablets of the composition shown in Table 3.

### EXAMPLE 5

### Formulation of 300 mg Tablet

The procedure described in Example 2 was used to manufacture tablets of the composition shown in Table 4.

### EXAMPLE 6

### Formulation of 400 mg Tablet

The procedure described in Example 2 was used to manufacture tablets of the composition shown in Table 5.

### EXAMPLE 7

### In Vitro Dissolution Assay—50 mg

### In Vitro Dissolution Protocol

The following method was used for ANN training, control of the formulations and as a predictor of in vivo release. The dissolution method is performed using the well-known basket apparatus at a rotation speed of 200 rpm. Initially, 900 mL of dissolution medium consisting of 0.05 M (molar) sodium citrate and 0.09 N (normal) sodium hydroxide are placed in each vessel. The pH of this medium is 4.8. At 5 hours, lp00 mL of a medium consisting of 0.05 M sodium phosphate and 0.46 N sodium hydroxide are added to each vessel to bring the pH of the medium to 6.6 for the final duration of the dissolution analysis. Samples are withdrawn over a 20 hour time-period and analyzed for quetiapine using ultraviolet spectrophotometric detection at 290 nm.

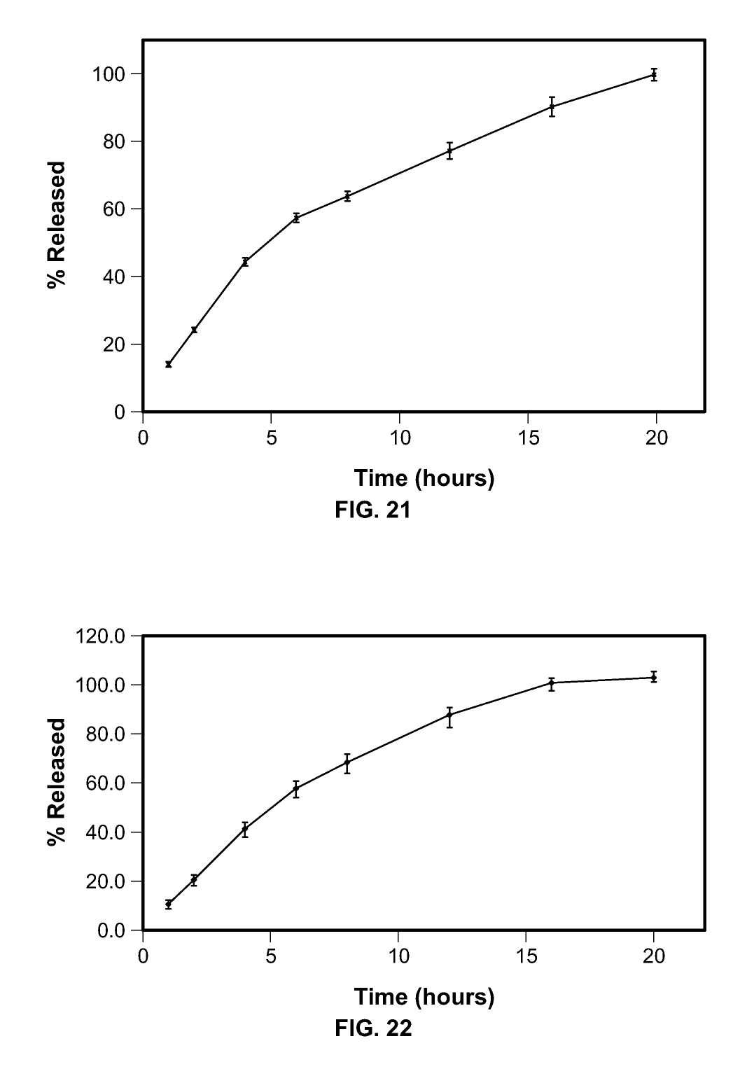

FIG. 21 shows the results of the dissolution assay. Error bars correspond to the range of the individual measurements at each time point.

### EXAMPLE 8

### In Vitro Dissolution Assay—150 mg

In vitro dissolution protocol performed as in Example (7). FIG. 22 shows the results of the dissolution assay. Error bars correspond to the range of the individual measurements at each time point.

### EXAMPLE 9

### In Vitro Dissolution Assay—200 mg

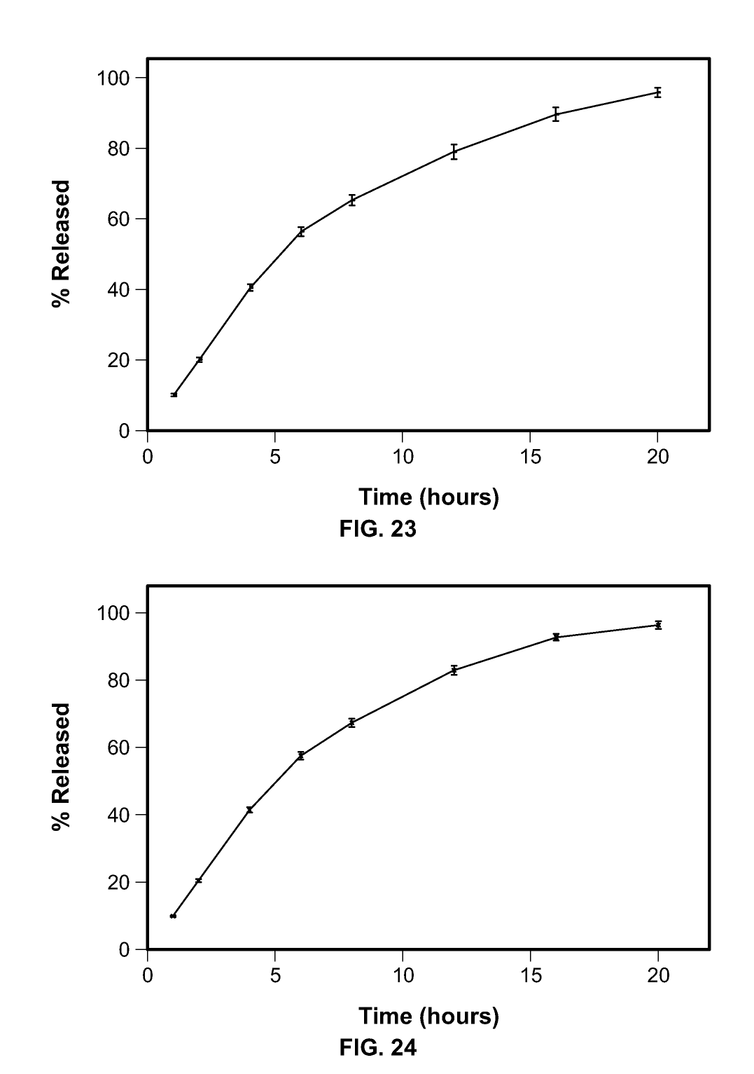

In vitro dissolution protocol performed as in Example (7). FIG. 23 shows the results of the dissolution assay. Error bars correspond to the range of the individual measurements at each time point.

### EXAMPLE 10

### In Vitro Dissolution Assay—300 mg

In vitro dissolution protocol performed as in Example (7). FIG. 24 shows the results of the dissolution assay. Error bars correspond to the range of the individual measurements at each time point.

### EXAMPLE 11

### In Vitro Dissolution Assay—400 mg

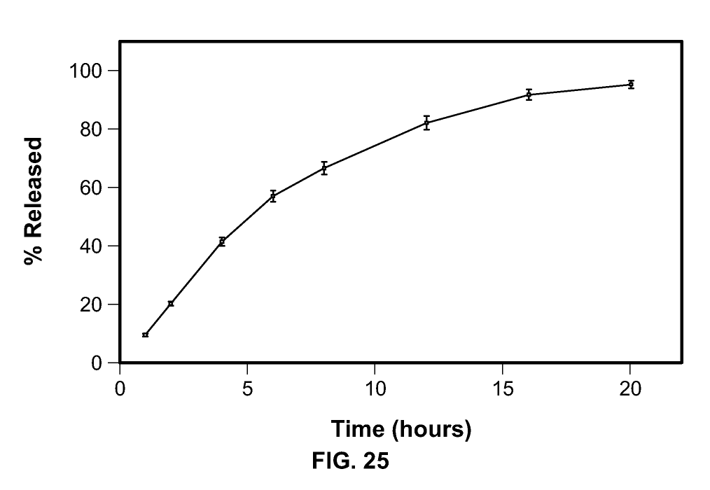

In vitro dissolution protocol performed as in Example (7). FIG. 25 shows the results of the dissolution assay. Error bars correspond to the range of the individual measurements at each time point.

### EXAMPLE 12

### Blood Plasma Protocol Studies

A multicenter, open-label, multiple-dose study was performed to evaluate the steady-state pharmacokinetics of commercial-scale tablets comprising study formulations (“SF”) having the following quetiapine strengths: 50 mg, 200 mg, 300 mg and 400 mg. The study formulations have compositions that are set forth in Tables 1-5. After a 2-day washout period, patients received oral doses of the study formulations and immediate-release (“IR”) medicament available under the trademark “Seroquel” (now available from AstraZeneca Pharmaceuticals, Wilmington, Del.) once daily as follows: 50 mg SF on Days 1 to 4, 200 mg SF on Days 5 to 7, 300 mg SF on Days 8 to 11, 400 mg SF on Days 12 to 14 and 300 mg IR on Days 15 to 17. On Days 4 and 11, patients consumed a standardized high-fat breakfast within 10 minutes of their scheduled dose. Data from Day 3 (50 mg; FIG. 3), Day 7 (200 mg; FIG. 4), Day 10 (300 mg; FIG. 5) and Day 14 (400 mg; FIG. 6) were used and it was assumed that steady-state had been achieved for each dose level. In each plot (FIGS. 3-6), bars correspond to the prediction interval (p=0.05) for individual subject data. Each plot (FIGS. 3-6) also shows a best fit curve calculated using first-order drug absorption and elimination rate constants Kₑ and Kₐ, respectively, with the equation

$$Y = base + ( \frac{1000 ( exp ( - {K}_{a} \times t ) - exp ( - {K}_{e} \times t ) )}{{K}_{e} / {K}_{a} - 1.5} ) .$$

The best fit parameters for the different tablet strengths are as follows:
50 mg: Base=0.3773; Kₑ=0.8421; Kₐ=0.05765 (FIG. 3)
200 mg: Base=25.86; Kₑ=0.3541; Kₐ=0.1033 (FIG. 4)
300 mg: Base=42.15; Kₑ=0.2592; Kₐ=0.1033 (FIG. 5)
400 mg: Base=62.96; Kₑ=0.2959; Kₐ=0.1390 (FIG. 6)

FIG. 7 shows data from FIGS. 3-6.

Thus, extended release formulations comprising quetiapine and its pharmaceutically acceptable salts and methods for manufacturing the formulations have been provided. Persons skilled in the art will appreciate that the invention may be practiced in the form of embodiments other than those described herein, which have been presented for purposes of illustration rather than limitation, and that the invention is limited only by the claims that follow.

### APPENDIX

TABLE A1-1
Time-point: 1
Layer: 1604 (See FIG. 16)

| Neuron | Bias (bⱼ) |
|---|---|
| j = 1 | 0.0862081 |
| j = 2 | −0.0716127 |
| j = 3 | −0.0364471 |
| j = 4 | 0.00349985 |
| j = 5 | −0.0285096 |
| j = 6 | −0.0607835 |
| j = 7 | 0.0258053 |
| j = 8 | −0.55539 |
| j = 9 | 0.0367189 |
| j = 10 | −0.0294554 |

TABLE A1-2
Time-point: 1
Layer: 1606 (see FIG. 16)

| Neuron | biasₒᵤₜₚᵤₜ |
|---|---|
| output | 0.174665 |

TABLE A1-3
Time-point: 1
Layer: 1604 (See FIG. 16)

| | |
|---|---|
| Number of neurons | 10 |

TABLE A1-4
Time-point: 1
Layer: 1606 (see FIG. 16)

| | |
|---|---|
| Number of neurons | 1 |

TABLE A1-5
Time-point: 1
Layer: 1604 (See FIG. 16)

| | Weight | | | | | |
|---|---|---|---|---|---|---|
| Neuron | i = 1 | i = 2 | i = 3 | i = 4 | i = 5 | i = 6 |
| j = 1 | 0.0587454 | 0.0501079 | −0.0401695 | 0.141361 | 0.0786457 | 0.101567 |
| j = 2 | −0.055606 | 0.0195879 | −0.00281477 | −0.0589501 | 0.0110449 | −0.0443374 |
| j = 3 | 0.121707 | −0.0362756 | 0.0181577 | 0.0218168 | 0.0543282 | −0.0619858 |
| j = 4 | 0.0339864 | −0.0206759 | −0.0514572 | −0.0343023 | 0.0279809 | 0.0315882 |
| j = 5 | −0.0521664 | 0.034939 | 0.037449 | −0.0135622 | −0.0535936 | 0.0501399 |
| j = 6 | 0.0911865 | −0.0221194 | 0.0412308 | 0.060883 | 0.00087899 | 0.0134231 |
| j = 7 | 0.00511157 | 0.00872833 | −0.0282108 | −0.0316863 | 0.0056049 | −0.0190538 |
| j = 8 | 0.153963 | −0.0676205 | −0.0816067 | −0.167644 | −0.0246992 | −0.14692 |
| j = 9 | 0.126428 | −0.00260182 | −0.000843538 | 0.14573 | 0.0309638 | −0.0262386 |
| j = 10 | −0.0243432 | 0.0267831 | 0.0339773 | −0.0467614 | −0.0187521 | −0.0325083 |
| | Weight | | | | | |
| Neuron | i = 7 | i = 8 | i = 9 | i = 10 | i = 11 | i = 12 |
| j = 1 | 0.0201306 | −0.00590794 | −0.0486776 | 0.00910753 | −0.00552561 | 0.011703 |
| j = 2 | 0.0333068 | −0.0155533 | 0.0447208 | −0.00944044 | 0.0116502 | 0.036764 |
| j = 3 | −0.0418341 | −0.0673008 | −0.0541343 | 0.0524179 | −0.0105394 | −0.0468912 |
| j = 4 | −0.0399084 | 0.0314295 | 0.0385104 | −0.0027799 | −0.0315197 | −0.0047047 |
| j = 5 | 0.046529 | 0.0546377 | 0.0200337 | 0.00396968 | 0.0512361 | 0.046391 |
| j = 6 | −0.0189535 | −0.0168035 | −0.0537568 | −0.0228817 | −0.0660598 | −0.030153 |
| j = 7 | 0.0487005 | −0.0240559 | 0.0277857 | −0.0415974 | −0.0284222 | 0.0153045 |
| j = 8 | 0.152526 | −0.0644217 | 0.073652 | 0.0509388 | 0.0729509 | −0.234367 |
| j = 9 | 0.00316126 | −0.088813 | −0.0712128 | 0.0814806 | −0.0916316 | 0.0502986 |
| j = 10 | 0.0360269 | 0.0204668 | −0.03585 | 0.0159227 | 0.0195872 | 0.00408231 |

| | Weight | | | | | |
|---|---|---|---|---|---|---|
| Neuron | i = 13 | i = 14 | i = 15 | i = 16 | i = 17 | i = 18 |
| j = 1 | 0.0311335 | −0.00277195 | −0.0775678 | −0.0308843 | 0.0309132 | −0.0443183 |
| j = 2 | 0.0377209 | 0.045552 | 0.00155676 | 0.0602104 | −0.0315647 | 0.0189457 |
| j = 3 | −0.049927 | 0.0423255 | 0.0238592 | 0.00244447 | 0.0492526 | −0.0117788 |
| j = 4 | 0.0229334 | 0.0421033 | −0.022445 | −0.0339328 | 0.0433137 | −0.0326121 |
| j = 5 | 0.0200194 | 0.0233769 | −0.0192553 | −0.0230639 | 0.0242465 | 0.0722059 |
| j = 6 | 0.0506873 | 0.00953144 | −0.0490043 | 0.0434506 | 0.170787 | −0.178531 |
| j = 7 | −0.0040824 | 0.0258253 | −2.88E−05 | −0.0423562 | −0.0171892 | −0.0209901 |
| j = 8 | 0.0320486 | 0.10309 | −0.0188616 | 0.0954643 | −0.0847904 | 0.0974787 |
| j = 9 | −0.0236111 | 0.00537207 | 0.0254217 | 0.0454215 | 0.0730283 | −0.0210851 |
| j = 10 | −0.00533041 | −0.0336704 | −0.0195659 | 0.0117876 | −0.0672437 | 0.0456167 |
| | Weight | | | | | |
| Neuron | i = 19 | i = 20 | i = 21 | i = 22 | i = 23 | i = 24 |
| j = 1 | −0.0660237 | 0.0568909 | 0.0754983 | −0.0458951 | −0.0422061 | −0.0336963 |
| j = 2 | 0.0451531 | −0.00347365 | −0.0220331 | 0.085815 | 0.0395822 | 0.056989 |
| j = 3 | −0.000729743 | −0.0164096 | 0.0249239 | 0.0399052 | −0.0503859 | 0.0457144 |
| j = 4 | −0.0322809 | −0.00774785 | −0.0388868 | 0.0177736 | 0.0241229 | −0.0180129 |
| j = 5 | 0.0155638 | 0.041108 | 0.050056 | 0.00795275 | 0.00780024 | 0.0121908 |
| j = 6 | 0.12627 | −0.055426 | −0.0849828 | 0.0775513 | 0.0344723 | 0.0613105 |
| j = 7 | −0.00469777 | 0.0297016 | −0.0204258 | −0.06674 | −0.033238 | 0.0106924 |
| j = 8 | −0.120833 | 0.073886 | 0.0587474 | −0.17048 | −0.0726205 | −0.0479177 |
| j = 9 | 0.00398679 | 0.0552127 | −0.0254888 | −0.0437598 | −0.0435234 | 0.00641966 |
| j = 10 | −0.0173692 | −0.0228823 | 0.0369229 | 0.00436892 | 0.0364141 | 0.0206152 |

TABLE A1-6
Time-point: 1
Layer: 1606 (see FIG. 16)

| | Neuron Weight | | | | |
|---|---|---|---|---|---|
| | j = 1 | j = 2 | j = 3 | j = 4 | j = 5 |
| output | 0.176894 | −0.0930464 | 0.146875 | 0.0309643 | −0.108632 |
| | Neuron Weight | | | | |
| | j = 6 | j = 7 | j = 8 | j = 9 | j = 10 |
| output | 0.24069 | −0.0203362 | 0.426691 | 0.246626 | −0.0738106 |

TABLE A2-1
Time-point: 2
Layer: 1604 (See FIG. 16)

| Neuron | Bias (bⱼ) |
|---|---|
| j = 1 | −0.0701742 |
| j = 2 | 0.0255841 |
| j = 3 | −0.0693046 |
| j = 4 | −0.0425284 |
| j = 5 | −0.115703 |
| j = 6 | 0.0576014 |
| j = 7 | 0.00689438 |
| j = 8 | 0.355753 |
| j = 9 | 0.0339459 |
| j = 10 | −0.0960811 |

TABLE A2-2
Time-point: 2
Layer: 1606 (see FIG. 16)

| Neuron | biasₒᵤₜₚᵤₜ |
|---|---|
| Output | −0.128571 |

TABLE A2-3
Time-point: 2
Layer: 1604 (See FIG. 16)

| | |
|---|---|
| Number of neurons | 10 |

TABLE A2-4
Time-point: 2
Layer: 1606 (see FIG. 16)

| | |
|---|---|
| Number of neurons | 1 |

TABLE A2-5
Time-point: 2
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | −0.05549 | −0.0241687 | 0.00841165 | −0.0550524 | 0.0376462 | −0.0116819 |
| j = 2 | −0.101987 | 0.0412675 | −0.00288371 | 0.00460335 | 0.00359613 | 0.0303261 |
| j = 3 | 0.0630521 | −0.087473 | −0.0174701 | −0.0274077 | 0.00144089 | −0.075456 |
| j = 4 | −0.070847 | −0.0401564 | −0.0295908 | −0.0211457 | 0.00516468 | −0.0304341 |
| j = 5 | −0.335513 | −0.0568648 | 0.118908 | 0.0610676 | 0.010292 | 0.0582923 |
| j = 6 | −0.0409421 | 0.0404524 | 0.014371 | 0.0112653 | −0.0285494 | −0.00518369 |
| j = 7 | 0.068651 | −0.0315162 | −0.0436239 | −0.0298333 | 0.0119764 | −0.0096256 |
| j = 8 | 0.0168323 | 0.0652212 | 0.0737072 | 0.20346 | 0.0519625 | 0.0924366 |
| j = 9 | 0.0329877 | −0.0214922 | −0.0165674 | −0.0232034 | −0.0082636 | 0.054114 |
| j = 10 | −0.110858 | 0.0159695 | −0.0274307 | −0.102876 | −0.0594375 | 0.00677716 |
| | Neuron Weight | | | | | |
| | i = 7 | i = 8 | i = 9 | i = 10 | i = 11 | i = 12 |
| j = 1 | 0.0137915 | 0.0492225 | 0.0507543 | −0.0404705 | 0.018028 | 0.0164064 |
| j = 2 | −0.0206612 | 0.0732916 | −0.0370291 | 0.0468979 | 0.000589027 | 0.0659562 |
| j = 3 | 0.0674336 | −0.00810405 | 0.00644726 | 0.00627442 | 0.0466169 | −0.0869203 |
| j = 4 | 0.0506304 | 0.0443839 | 0.0249216 | 0.0418441 | 0.0472165 | 0.0619423 |
| j = 5 | −0.0548188 | 0.0319274 | −0.018853 | −0.0491261 | −0.0530881 | 0.0890952 |
| j = 6 | 0.0172181 | −0.0044099 | −0.0146831 | 0.0274924 | 0.000841134 | 0.0988664 |
| j = 7 | −0.0180228 | −0.0428003 | −0.049244 | −0.00133295 | 0.00612159 | −0.00482974 |
| j = 8 | −0.0585251 | −0.0755541 | −0.144472 | 0.0344791 | −0.166187 | 0.0668326 |
| j = 9 | 0.0367966 | 0.0122304 | 0.035599 | −0.04166 | −0.0170463 | −0.013241 |
| j = 10 | −0.0391209 | 0.0292828 | 0.0673243 | −0.0256494 | 0.0498498 | −0.000558756 |
| | Neuron Weight | | | | | |
| | i = 13 | i = 14 | i = 15 | i = 16 | i = 17 | i = 18 |
| j = 1 | 0.0328864 | 0.0403878 | −0.00853128 | 0.0140241 | −0.0490749 | 0.0475306 |
| j = 2 | −0.00233216 | 0.00951423 | 0.0467018 | −0.0850964 | −0.0256714 | 0.0528631 |
| j = 3 | 0.0750499 | 0.00572479 | 0.00710286 | 0.120597 | 0.065206 | 0.0014325 |
| j = 4 | −0.00889632 | 0.0509869 | 0.0307375 | 0.0121623 | 0.00061679 | 0.00125828 |
| j = 5 | −0.0354271 | −0.165374 | −0.0485568 | −0.0480192 | −0.0244288 | −0.00971913 |
| j = 6 | −0.00298766 | −0.0360945 | 0.0103312 | −0.0261382 | −0.0820488 | 0.103207 |
| j = 7 | −0.0416729 | −0.000182291 | −0.0427128 | 0.0442638 | 0.020318 | −0.0457548 |
| j = 8 | −0.0849963 | −0.0770615 | −0.0875602 | −0.0537549 | 0.0922173 | −0.135128 |
| j = 9 | −0.0479105 | −0.00848021 | −0.0300716 | 0.0338161 | −0.0103057 | 0.0425586 |
| j = 10 | −0.0513309 | −0.0340473 | 0.0431609 | −0.0102862 | −0.0429171 | 0.0603725 |
| | Neuron Weight | | | | | |
| | i = 19 | i = 20 | i = 21 | i = 22 | i = 23 | i = 24 |
| j = 1 | 0.0849901 | −0.0762371 | −0.00537404 | 0.0889996 | 0.0267541 | −0.0187775 |
| j = 2 | −0.0432972 | −0.0111991 | −0.0131274 | 0.0121061 | −0.00114753 | −0.0579 |
| j = 3 | 0.0264153 | −0.00138583 | −0.00588905 | −0.0460385 | 0.0440498 | −0.0203203 |
| j = 4 | −0.0189386 | −0.0726018 | 0.0172797 | 0.052222 | 0.062901 | 0.0480173 |
| j = 5 | −0.113172 | 0.0859503 | 0.11728 | −0.0218165 | −0.0434255 | −0.0716188 |
| j = 6 | −0.00640902 | 0.066877 | 0.00283008 | −0.045821 | 0.0105698 | −0.0135852 |
| j = 7 | −0.0380425 | 0.0385532 | 0.0321388 | −0.0190822 | −0.0733301 | −0.0584756 |
| j = 8 | −0.148695 | 0.176065 | 0.152279 | −0.271038 | −0.163519 | −0.036839 |
| j = 9 | −0.0323205 | 0.0982542 | 0.057129 | −0.094514 | −0.0116676 | −0.00762018 |
| j = 10 | 0.0284044 | −0.0725574 | −0.0549959 | 0.102135 | 0.0514465 | 0.0407291 |

TABLE A2-6
Time-point: 2
Layer: 1606 (see FIG. 16)

| | Neuron Weight | | | | |
|---|---|---|---|---|---|
| | j = 1 | j = 2 | j = 3 | j = 4 | j = 5 |
| output | −0.0782786 | −0.179101 | 0.21993 | −0.0408936 | −0.431685 |
| | Neuron Weight | | | | |
| | j = 6 | j = 7 | j = 8 | j = 9 | j = 10 |
| output | −0.127344 | 0.0478728 | 0.429125 | −0.0487826 | −0.173632 |

TABLE A3-1
Time-point: 3
Layer: 1604 (See FIG. 16)

| Neuron | Bias (bⱼ) |
|---|---|
| j = 1 | 0.0269206 |
| j = 2 | 0.00454842 |
| j = 3 | −0.0258995 |
| j = 4 | 0.0366767 |
| j = 5 | −0.191506 |
| j = 6 | 0.0480326 |
| j = 7 | −0.011048 |
| j = 8 | 0.00919401 |
| j = 9 | −0.0188623 |
| j = 10 | 0.15127 |

TABLE A3-2
Time-point: 3
Layer: 1606 (see FIG. 16)

| Neuron | biasₒᵤₜₚᵤₜ |
|---|---|
| Output | 0.0393023 |

TABLE A3-3
Time-point: 3
Layer: 1604 (See FIG. 16)

| | |
|---|---|
| Number of neurons | 10 |

TABLE A3-4
Time-point: 3
Layer: 1606 (see FIG. 16)

| | |
|---|---|
| Number of neurons | 1 |

TABLE A3-5
Time point: 3
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | −0.0761845 | −0.00705585 | 0.0169188 | 0.0352893 | 0.0296078 | 0.0516466 |
| j = 2 | −0.024696 | −0.037836 | 0.0288388 | −0.0395627 | 0.000171833 | 0.0565581 |
| j = 3 | 0.0353958 | 0.0346459 | 0.0312341 | −0.0589405 | −0.0432113 | 0.0062965 |
| j = 4 | −0.176395 | −0.0150413 | −0.00158872 | 0.0609651 | 0.00525665 | 0.021398 |
| j = 5 | −0.00113048 | −0.0605293 | −0.0332388 | −0.13621 | −0.093746 | −0.103189 |
| j = 6 | −0.251726 | 0.0491608 | 0.0396731 | 0.0731025 | −0.0328528 | 0.141794 |
| j = 7 | 0.122749 | −0.0236897 | −0.0337763 | 0.0267044 | −0.00879349 | −0.00247985 |
| j = 8 | −0.0949875 | −0.00689759 | 0.0135027 | 0.0478375 | 0.00305713 | 0.0548459 |
| j = 9 | 0.0255457 | −0.0543757 | 0.0295072 | −0.017764 | 0.00632454 | −0.0485704 |
| j = 10 | −0.27669 | −0.0753402 | −0.008663 | −0.0912977 | −0.0341727 | 0.00689565 |
| | Neuron Weight | | | | | |
| | i = 7 | i = 8 | i = 9 | i = 10 | i = 11 | i = 12 |
| j = 1 | −0.0275926 | 0.0124496 | 0.0134113 | 0.027417 | −0.0141111 | 0.0574973 |
| j = 2 | −0.0291299 | −0.0181403 | 0.0178527 | 0.0151396 | −0.0264879 | −0.0421513 |
| j = 3 | −0.0294799 | 0.0144908 | −0.00452842 | 0.00924955 | −0.0232185 | 0.0184786 |
| j = 4 | −0.081213 | 0.0270382 | −0.000364408 | −0.00622683 | −0.0184549 | 0.061207 |
| j = 5 | −0.0103789 | 0.102193 | 0.15164 | 0.011826 | 0.089355 | −0.174425 |
| j = 6 | −0.0245589 | 0.0581496 | −0.0562678 | 0.00144571 | −0.0307063 | 0.0589873 |
| j = 7 | 0.051225 | −0.093029 | 0.0352704 | 0.013688 | 0.0302193 | −0.0282144 |
| j = 8 | −0.0334224 | 0.0871717 | −0.045059 | 0.0427439 | 0.0214819 | −0.00204501 |
| j = 9 | 0.0158051 | 0.0110144 | 0.0474333 | −0.00234056 | 0.0587813 | 0.013508 |
| j = 10 | 0.0851242 | −0.0849504 | 0.181423 | −0.0757893 | 0.111695 | 0.0805882 |
| | Neuron Weight | | | | | |
| | i = 13 | i = 14 | i = 15 | i = 16 | i = 17 | i = 18 |
| j = 1 | 0.023296 | −0.0369768 | −0.0273763 | −0.0189211 | 0.0481931 | −0.0132605 |
| j = 2 | −0.0364437 | 5.11E−05 | −0.00805075 | 0.00276521 | −0.00695709 | −0.00674528 |
| j = 3 | −0.0134613 | 0.0150502 | 0.0404254 | −0.000938222 | 0.000254447 | 0.00542848 |
| j = 4 | −0.0182518 | −0.0115655 | −0.040195 | −0.0868905 | −0.00036936 | −0.0421035 |
| j = 5 | 0.0782033 | −0.00765612 | 0.0750009 | 0.136332 | −0.0442229 | 0.019199 |
| j = 6 | −0.0151374 | −0.0233836 | 0.0216421 | −0.0389496 | 0.0159675 | −0.01094 |
| j = 7 | −0.0167602 | 0.01955 | 0.0284668 | 0.0356969 | 0.00560458 | 0.0143632 |
| j = 8 | −0.0235624 | −0.0314985 | 0.0191205 | 0.00156102 | −0.0234805 | 0.0235205 |
| j = 9 | −0.0510385 | 0.000998289 | 0.0485551 | 0.0547664 | 0.0298506 | −0.00866584 |
| j = 10 | −0.0432113 | 0.0885201 | 0.0980645 | 0.0735889 | −0.427368 | 0.394866 |
| | Neuron Weight | | | | | |
| | i = 19 | i = 20 | i = 21 | i = 22 | i = 23 | i = 24 |
| j = 1 | 0.00900439 | −0.0524896 | −0.008703 | 0.0293718 | −0.0215677 | 0.0385262 |
| j = 2 | 0.00334374 | −0.0222879 | −0.047919 | 0.0511491 | −0.0195292 | 0.0512658 |
| j = 3 | −0.0562554 | 0.0274223 | 0.0494387 | −0.0146217 | −0.0549827 | 0.0174912 |
| j = 4 | 0.0372967 | 0.0366507 | 0.0258917 | 0.0294151 | 0.0118108 | 0.0219255 |
| j = 5 | 0.0776932 | −0.181402 | −0.181516 | 0.246002 | 0.17777 | 0.0497206 |
| j = 6 | −0.0393345 | 0.0190678 | 0.0530362 | 0.0675038 | −0.0225341 | −0.0176436 |
| j = 7 | 0.0287077 | 0.0286062 | −0.0221776 | −0.0490121 | −0.0664462 | −0.0117451 |
| j = 8 | 0.00271584 | −0.0286797 | 0.0310103 | 0.0198695 | −0.0223976 | 0.0244137 |
| j = 9 | −0.0275525 | −0.0174597 | −0.0383274 | 0.0303165 | −0.00634466 | −0.0288927 |
| j = 10 | −0.105468 | 0.128608 | 0.0882429 | −0.0923618 | 0.0264514 | −0.00456059 |

TABLE A3-6
Time-point: 3
Layer: 1606 (see FIG. 16)

| | Neuron Weight | | | | |
|---|---|---|---|---|---|
| | i = 1 | i = 2 | j = 3 | j = 4 | j = 5 |
| output | −0.117215 | −0.0347292 | 0.0326048 | −0.212831 | −0.35674 |
| | Neuron Weight | | | | |
| | j = 6 | j = 7 | j = 8 | j = 9 | j = 10 |
| output | −0.307658 | 0.172289 | −0.110608 | 0.0525908 | −0.429422 |

TABLE A4-1
Time-point: 4
Layer: 1604 (See FIG. 16)

| Neuron | Bias (bⱼ) |
|---|---|
| j = 1 | 0.0501398 |
| j = 2 | −0.0382328 |
| j = 3 | −0.0856816 |
| j = 4 | 0.236611 |
| j = 5 | 0.0212253 |
| j = 6 | 0.0416355 |
| j = 7 | 0.0284566 |
| j = 8 | 0.0154394 |
| j = 9 | −0.125146 |
| j = 10 | −0.0018378 |

TABLE A4-2
Time-point: 4
Layer: 1606 (see FIG. 16)

| Neuron | biasₒᵤₜₚᵤₜ |
|---|---|
| Output | 0.00942657 |

TABLE A4-3
Time-point: 4
Layer: 1604 (See FIG. 16)

| | |
|---|---|
| Number of neurons | 10 |

TABLE A4-4
Time-point: 4
Layer: 1606 (see FIG. 16)

| | |
|---|---|
| Number of neurons | 1 |

TABLE A4-5
Time-point: 4
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | −0.169409 | 0.0479363 | 0.0236194 | −0.00584937 | 0.0216985 | 0.0463525 |
| j = 2 | −0.268873 | 0.00425418 | 0.0408584 | 0.0582653 | 0.0335634 | 0.0581736 |
| j = 3 | 0.18869 | 0.0401782 | −0.0232596 | −0.0110809 | 0.018416 | −0.0583009 |
| j = 4 | 0.0434695 | 0.112777 | 0.0678226 | 0.123909 | 0.112728 | 0.0908078 |
| j = 5 | −0.0496167 | 0.0161506 | 0.0191324 | −0.0326416 | −0.0342273 | 0.0458854 |
| j = 6 | −0.0756907 | 0.00491072 | 0.0495982 | 0.0498021 | 0.00571049 | −0.019929 |
| j = 7 | 0.0882509 | −0.00504873 | −0.0112633 | −0.0258551 | −0.00850263 | −0.033622 |
| j = 8 | 0.0538439 | 0.0328055 | −0.0409804 | 0.00928505 | −0.0274688 | −0.0265463 |
| j = 9 | 0.307696 | 0.0547152 | −0.00484539 | 0.070701 | 0.0304405 | −0.0505936 |
| j = 10 | 0.0173676 | 0.0314775 | 0.022969 | −0.0325141 | −0.0411061 | 0.0476734 |
| | Neuron Weight | | | | | |
| | i = 7 | i = 8 | i = 9 | i = 10 | i = 11 | i = 12 |
| j = 1 | −0.0290208 | 0.050501 | 0.0364252 | 0.0440355 | −0.0521916 | 0.00997111 |
| j = 2 | −0.0610999 | 0.0667456 | −0.0388303 | −0.0562703 | −0.000161768 | 0.0222207 |
| j = 3 | −0.047982 | 0.0302945 | −0.0954631 | 0.0368086 | −0.0233822 | −0.058165 |
| j = 4 | 0.0251979 | −0.0918976 | −0.165977 | −0.0453814 | −0.105506 | 0.103507 |
| j = 5 | −0.0182855 | 0.0706746 | −0.0360076 | 0.0174732 | 0.0201701 | 0.00955145 |
| j = 6 | 0.0269958 | 0.0106582 | −0.0284321 | 0.0238198 | −0.0618295 | 0.0365542 |
| j = 7 | 0.0863603 | −0.0809485 | −0.0336661 | 0.0219785 | 0.0317752 | −0.0769041 |
| j = 8 | 0.011201 | 0.0153545 | −0.00725413 | −0.00576001 | 0.0523462 | −0.0342063 |
| j = 9 | −0.0543062 | 0.0525467 | −0.0984304 | 0.0454458 | −0.0810539 | −0.0327171 |
| j = 10 | −0.0123966 | −0.0120837 | 0.0346122 | −0.0440171 | 0.0176828 | −0.0448068 |
| | Neuron Weight | | | | | |
| | i = 13 | i = 14 | i = 15 | i = 16 | i = 17 | i = 18 |
| j = 1 | 0.0243549 | −0.0291709 | −0.0252644 | −0.0544641 | 0.0506076 | −0.021149 |
| j = 2 | −0.0588519 | −0.0387293 | 0.00575227 | −0.0148377 | −0.0116889 | −0.0273424 |
| j = 3 | 0.0119067 | −0.0253309 | −0.022094 | −0.0389479 | 0.215361 | −0.182309 |
| j = 4 | −0.0873589 | 0.0302363 | −0.0995788 | −0.145962 | 0.0628528 | −0.0684247 |
| j = 5 | −0.0162817 | −0.0189364 | −0.0149927 | −0.0656979 | −0.037444 | −0.0531075 |
| j = 6 | −0.0117661 | 0.0372181 | −0.0298497 | 0.021301 | 0.0376582 | 0.0519655 |
| j = 7 | −0.0204208 | 0.0193044 | 0.00803842 | 0.0282743 | 0.0369261 | −0.00814179 |
| j = 8 | 0.0337991 | −0.0495765 | 0.0281412 | −0.0363734 | 0.00533613 | −0.0139108 |
| j = 9 | 0.0340369 | −0.100396 | −0.0687517 | −0.0279417 | 0.312328 | −0.278621 |
| j = 10 | −0.0384001 | −0.00782981 | 0.0401185 | 0.0574896 | −0.0510635 | 0.0446248 |
| | Neuron Weight | | | | | |
| | i = 19 | i = 20 | i = 21 | i = 22 | i = 23 | i = 24 |
| j = 1 | −0.0209428 | −0.0171858 | −0.0134748 | 0.0420275 | 0.0666227 | 0.0369626 |
| j = 2 | −0.0542291 | 0.0344592 | 0.0487627 | 0.0243467 | −0.0138488 | −0.0670747 |
| j = 3 | 0.134243 | −0.0684568 | −0.0651444 | 0.097978 | 0.0101173 | 0.0873532 |
| j = 4 | −0.175177 | 0.179334 | 0.167438 | −0.236263 | −0.207708 | −0.0947039 |
| j = 5 | −0.00884843 | −0.00707117 | −0.0345188 | 0.0276125 | 0.026459 | −0.0214315 |
| j = 6 | 0.0174585 | −0.0128738 | −0.0360703 | 0.0288861 | 0.0348711 | 0.0542564 |
| j = 7 | −0.035106 | 0.00802213 | 0.0276039 | 0.0104067 | 0.00154075 | −0.00646086 |
| j = 8 | 0.0232698 | 0.00215175 | 0.0222038 | −0.00681416 | −0.0219162 | −0.0336228 |
| j = 9 | 0.0975082 | −0.0723264 | −0.136951 | 0.0757903 | −0.0300047 | 0.0597008 |
| j = 10 | 0.0248781 | −0.026328 | 0.0307545 | 0.0121026 | −0.0205361 | 0.0342049 |

TABLE A4-6
Time-point: 4
Layer: 1606 (see FIG. 16)

| | Neuron Weight | | | | |
|---|---|---|---|---|---|
| | j = 1 | j = 2 | j = 3 | j = 4 | j = 5 |
| output | −0.219576 | −0.243772 | 0.205451 | 0.37336 | −0.0879003 |
| | Neuron Weight | | | | |
| | j = 6 | j = 7 | j = 8 | j = 9 | j = 10 |
| output | −0.0810799 | 0.116659 | 0.0434714 | 0.352234 | 0.0674042 |

TABLE A5-1
Time-point: 5
Layer: 1604 (See FIG. 16)

| Neuron | Bias (bⱼ) |
|---|---|
| j = 1 | 0.0125852 |
| j = 2 | −0.0313829 |
| j = 3 | −0.0225728 |
| j = 4 | −0.014204 |
| j = 5 | 0.0489281 |
| j = 6 | 0.0721708 |
| j = 7 | 0.0665779 |
| j = 8 | −0.0512846 |
| j = 9 | 0.0186739 |
| j = 10 | −0.0548022 |

TABLE A5-2
Time-point: 5
Layer: 1606 (see FIG. 16)

| Neuron | biasₒᵤₜₚᵤₜ |
|---|---|
| Output | 0.0324696 |

TABLE A5-3
Time-point: 5
Layer: 1604 (See FIG. 16)

| | |
|---|---|
| Number of neurons | 10 |

TABLE A5-4
Time-point: 5
Layer: 1606 (see FIG. 16)

| | |
|---|---|
| Number of neurons | 1 |

TABLE A5-5
Time-point: 5
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | −0.117837 | −0.0115581 | 0.000329315 | −0.132637 | −0.0103362 | 0.0779523 |
| j = 2 | 0.0263014 | −0.0267303 | −0.0191103 | −0.00900318 | −0.030963 | −0.0082193 |
| j = 3 | 0.0951506 | 0.0046457 | 0.0445969 | −0.000123563 | −0.0121782 | −0.0519546 |
| j = 4 | −0.0437677 | −0.00544788 | −0.0422712 | −0.0227841 | −0.0602167 | 0.055929 |
| j = 5 | −0.319441 | −0.118199 | 0.0550908 | −0.0440541 | −0.0538592 | −0.0268005 |
| j = 6 | −0.197348 | 0.0682249 | −0.0238562 | 0.0740576 | −0.0264477 | 0.0266055 |
| j = 7 | 0.291665 | 0.110481 | 0.036893 | −0.0710762 | 0.0447239 | −0.083299 |
| j = 8 | 0.197773 | −0.06256 | −0.0342679 | 0.0348734 | 0.0768228 | −0.00262207 |
| j = 9 | 0.0464801 | 0.0343134 | 0.0273995 | −0.0185155 | 0.0305295 | 0.00698746 |
| j = 10 | 0.144877 | 0.0580166 | 0.040314 | −0.0492264 | −0.0103701 | −0.0430387 |
| | Neuron Weight | | | | | |
| | i = 7 | i = 8 | i = 9 | i = 10 | i = 11 | i = 12 |
| j = 1 | 0.0462593 | 0.0346336 | 0.081517 | 0.00244001 | −0.00537829 | −0.0925792 |
| j = 2 | 0.0538925 | −0.0541204 | −0.00948887 | −0.037629 | −0.0143762 | −0.0496724 |
| j = 3 | −0.00912976 | −0.0575188 | 0.0163969 | −0.0284097 | 0.00923708 | 0.0219293 |
| j = 4 | 0.00506943 | 0.024996 | 0.048639 | −0.0101329 | −0.0425672 | 0.0322479 |
| j = 5 | 0.0434162 | −0.0935664 | 0.183059 | −0.0847384 | 0.177259 | 0.0034615 |
| j = 6 | −0.0847134 | 0.072704 | 0.0427085 | 0.0236824 | 0.0150611 | 0.038672 |
| j = 7 | 0.00441522 | −0.0514565 | 0.0384803 | 0.0164271 | 0.0355876 | −0.0149905 |
| j = 8 | 0.0257839 | −0.0426909 | −0.0472011 | −0.0311973 | 0.0174797 | 0.0213192 |
| j = 9 | 0.00840099 | 0.0139316 | −0.00349568 | −0.00495638 | 0.0419534 | −0.000297343 |
| j = 10 | 0.0263483 | −0.0035801 | −0.0315152 | 0.0209558 | −0.0216988 | −0.0475047 |
| | Neuron Weight | | | | | |
| | i = 13 | i = 14 | i = 15 | i = 16 | i = 17 | i = 18 |
| j = 1 | 0.0431318 | 0.0167042 | 0.0261303 | 0.0792962 | −0.0223161 | −0.0054535 |
| j = 2 | −0.0517494 | −0.0219651 | 0.0242333 | 0.068377 | −0.0449256 | 0.0201732 |
| j = 3 | 0.01604 | −0.0546365 | −0.0200082 | 0.0032793 | −0.0372823 | −0.0216101 |
| j = 4 | −0.0143626 | 0.017505 | −0.00508514 | −0.0253677 | 0.0158434 | −0.0102422 |
| j = 5 | −0.0584057 | 0.0660546 | 0.107758 | 0.0702672 | −0.508955 | 0.463704 |
| j = 6 | 0.0742032 | 0.00263047 | 0.023944 | −0.0111704 | 0.0590508 | −0.0345178 |
| j = 7 | −0.000612696 | 0.0213692 | 0.0175423 | −0.0224854 | −0.0164412 | −0.0284589 |
| j = 8 | −0.0675792 | 0.0160937 | −1.32E−05 | −0.0654642 | −0.0183994 | 0.0257412 |
| j = 9 | −0.0456027 | 0.00239401 | 0.0317165 | −0.0404531 | −0.0425018 | −0.0354072 |
| j = 10 | −0.0159884 | −0.0652141 | 0.0367528 | 0.0233864 | 0.0568041 | −0.0343291 |
| | Neuron Weight | | | | | |
| | i = 19 | i = 20 | i = 21 | i = 22 | i = 23 | i = 24 |
| j = 1 | 0.079646 | −0.1084 | −0.10983 | 0.12879 | 0.0578103 | 0.0770606 |
| j = 2 | 0.00367161 | 0.0587732 | −0.0219005 | −0.0627764 | −0.0243308 | 0.028225 |
| j = 3 | −0.00718821 | 0.0427485 | 0.00674583 | −0.0448986 | −0.0790802 | 0.0239859 |
| j = 4 | 0.0463342 | 0.0138033 | −0.00977628 | 0.0892427 | 0.0763259 | 0.017539 |
| j = 5 | −0.140458 | 0.0824789 | 0.144054 | −0.145494 | −0.0338046 | 0.115637 |
| j = 6 | 0.037095 | −0.0124728 | −0.0170645 | 0.0498195 | 0.0738262 | −0.0200075 |
| j = 7 | 0.106717 | −0.0632294 | −0.0807679 | −0.0291343 | −0.00250981 | 0.0883975 |
| j = 8 | −0.0130429 | 0.0151362 | 0.0219933 | −0.104942 | −0.0858266 | −0.0570673 |
| j = 9 | −0.058092 | −0.0232588 | 0.0705049 | −0.0557213 | −0.0666165 | −0.00626976 |
| j = 10 | 0.000356535 | −1.64E−05 | 0.00775384 | 0.00697605 | −0.0080759 | −0.00627241 |

TABLE A5-6
Time-point: 5
Layer: 1606 (see FIG. 16)

| | Neuron Weight | | | | |
|---|---|---|---|---|---|
| | j = 1 | j = 2 | j = 3 | j = 4 | j = 5 |
| output | −0.203793 | 0.121035 | 0.103872 | −0.103158 | −0.406568 |
| | Neuron Weight | | | | |
| | j = 6 | j = 7 | j = 8 | j = 9 | j = 10 |
| output | −0.260208 | 0.242492 | 0.229785 | 0.0842838 | 0.0812001 |

TABLE A6-1
Time-point: 6
Layer: 1604 (See FIG. 16)

| Neuron | Bias (bⱼ) |
|---|---|
| j = 1 | 0.101109 |
| j = 2 | 0.0267049 |
| j = 3 | −0.00510953 |
| j = 4 | 0.0400461 |
| j = 5 | 0.0168025 |
| j = 6 | −0.0306495 |
| j = 7 | −0.0233148 |
| j = 8 | −0.0311746 |
| j = 9 | 0.0201758 |
| j = 10 | −0.0341079 |

TABLE A6-2
Time-point: 6
Layer: 1606 (see FIG. 16)

| Neuron | biasₒᵤₜₚᵤₜ |
|---|---|
| output | −0.000605426 |

TABLE A6-3
Time-point: 6
Layer: 1604 (See FIG. 16)

| | |
|---|---|
| Number of neurons | 10 |

TABLE A6-4
Time-point: 6
Layer: 1606 (See FIG. 16)

| | |
|---|---|
| Number of neurons | 1 |

TABLE A6-5
Time-point: 6
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | −0.221655 | −0.0034164 | 0.0464695 | −0.0606163 | −0.050726 | 0.111161 |
| j = 2 | −0.0554215 | −0.0327107 | −0.00481696 | 0.0267779 | −0.0260359 | 0.013998 |
| j = 3 | 0.137458 | −0.0279178 | 0.0170828 | −0.043786 | 0.024689 | −0.08217 |
| j = 4 | −0.160348 | −0.0161393 | 0.0166395 | 0.0363286 | −0.0133584 | 0.0646177 |
| j = 5 | −0.0934942 | 0.00366727 | 0.0164927 | −0.0461432 | −0.0273684 | −0.0181134 |
| j = 6 | 0.0367831 | 0.0298052 | 0.0494654 | −0.0145117 | −0.0309604 | −0.0288948 |
| j = 7 | −0.121522 | 0.00849915 | 0.0318361 | −0.00639512 | −0.0928455 | 0.0427896 |
| j = 8 | −0.21548 | 0.0548589 | −0.0508047 | 0.0682523 | −0.0616401 | −0.0105167 |
| j = 9 | 0.139569 | 0.00883309 | −0.00337677 | −0.021431 | 0.0615947 | −0.0292313 |
| j = 10 | −0.456249 | −0.16682 | −0.0167462 | 0.00891838 | 0.0102707 | 0.0523526 |
| | Neuron Weight | | | | | |
| | i = 7 | i = 8 | i = 9 | i = 10 | i = 11 | i = 12 |
| j = 1 | −0.0502136 | 0.0432468 | 0.0988992 | −0.0334365 | −0.0296078 | −0.0295228 |
| j = 2 | −0.0073953 | −0.0221787 | 0.00421413 | −0.0215401 | 0.000104699 | −0.0316712 |
| j = 3 | 0.100801 | −0.0444617 | −0.0326853 | −0.0761431 | 0.0375643 | −0.0806662 |
| j = 4 | 0.0350114 | −0.000425223 | −0.0184253 | −0.0379567 | 0.0411886 | −0.0427435 |
| j = 5 | −0.0703129 | 0.030225 | 0.0408648 | 0.0478728 | 0.0131197 | −0.0421979 |
| j = 6 | −0.0260397 | −0.0602544 | 0.0352687 | 0.0102832 | −0.0219795 | −0.0138111 |
| j = 7 | −0.0632111 | 0.0796905 | 0.038806 | 0.0048893 | 0.00567263 | −0.0159725 |
| j = 8 | −0.0528358 | 0.0530705 | 0.0285268 | 0.0334161 | 0.02902 | −0.0143393 |
| j = 9 | 0.0503761 | −0.0352819 | −0.0441687 | −0.0659964 | 0.0107697 | −0.0301924 |
| j = 10 | 0.194188 | −0.0869855 | 0.132103 | −0.169519 | 0.0880142 | −0.0226581 |
| | Neuron Weight | | | | | |
| | i = 13 | i = 14 | i = 15 | i = 16 | i = 17 | i = 18 |
| j = 1 | 0.0894137 | 0.0819942 | 0.0103954 | 0.0757638 | 0.00607849 | −0.00705411 |
| j = 2 | −0.0260188 | 0.0176401 | −0.051856 | −0.00571188 | 0.00492948 | 0.0599527 |
| j = 3 | −0.0013695 | 0.0329998 | −0.00492968 | 0.0320101 | 0.0261765 | −0.0165987 |
| j = 4 | 0.0772444 | 0.036077 | −0.0105357 | −0.00324233 | −0.0564957 | 0.042341 |
| j = 5 | 0.0666505 | 0.0163816 | 0.0140645 | 0.0307426 | 0.0377274 | −0.038838 |
| j = 6 | 0.0210592 | −0.0575944 | −0.0365472 | 0.0339347 | −0.0250761 | −0.0270859 |
| j = 7 | −0.00251443 | 0.0327636 | −0.0317766 | 0.0137035 | 0.0220997 | 0.0257468 |
| j = 8 | 0.022444 | 0.0145441 | −0.061312 | −0.00135096 | −0.0279067 | −0.0283411 |
| j = 9 | −0.0303319 | −0.022803 | 0.0532039 | −0.0310606 | 0.0131452 | −0.0310003 |
| j = 10 | 0.00377543 | 0.0943244 | 0.168774 | 0.172206 | −0.447492 | 0.400499 |
| | Neuron Weight | | | | | |
| | i = 19 | i = 20 | i = 21 | i = 22 | i = 23 | i = 24 |
| j = 1 | 0.0220793 | −0.0751295 | 0.01513 | 0.0928405 | 0.0910709 | −0.0118122 |
| j = 2 | −0.00701743 | −0.0649244 | 0.0171217 | 0.0371398 | 0.0490496 | 0.0545453 |
| j = 3 | −0.0104019 | −0.00105614 | 0.0544316 | −0.0839354 | −0.0175544 | −0.0439771 |
| j = 4 | −0.00242994 | 0.00897247 | −0.0308642 | 0.00961589 | −0.00948557 | 0.0363285 |
| j = 5 | 0.0714093 | 0.00333306 | −0.0781684 | 0.0844184 | 0.00369618 | 0.0110791 |
| j = 6 | −0.0165475 | −0.0277176 | 0.0003911 | −0.030471 | −0.0381136 | 0.0179558 |
| j = 7 | −0.00377792 | −0.014853 | −0.0457184 | 0.0801542 | 0.0346383 | 0.0157196 |
| j = 8 | −0.0363599 | −0.00779024 | 0.0124047 | 0.0208768 | 0.0360567 | −0.0151167 |
| j = 9 | −0.00416452 | 0.00397458 | 0.0268947 | −0.0628434 | −0.0682341 | −0.0498935 |
| j = 10 | −0.12551 | 0.140454 | 0.140946 | −0.0931052 | 0.0211132 | 0.0463931 |

TABLE A6-6
Time-point: 6
Layer: 1606 (see FIG. 16)

| | Neuron Weight | | | | |
|---|---|---|---|---|---|
| | j = 1 | j = 2 | j = 3 | j = 4 | j = 5 |
| output | −0.241895 | −0.0252866 | 0.182008 | −0.0663759 | −0.157681 |
| | Neuron Weight | | | | |
| | j = 6 | j = 7 | j = 8 | j = 9 | j = 10 |
| output | 0.0202451 | −0.164701 | −0.199368 | 0.149083 | −0.40854 |

TABLE A7-1
Time-point: 7
Layer: 1604 (See FIG. 16)

| Neuron | Bias (bⱼ) |
|---|---|
| j = 1 | 0.0687325 |
| j = 2 | 0.270454 |
| j = 3 | −0.0410544 |
| j = 4 | 0.0117681 |
| j = 5 | 0.0376671 |
| j = 6 | −0.339399 |
| j = 7 | −0.384568 |
| j = 8 | 0.00231177 |
| j = 9 | 0.023333 |
| j = 10 | −0.00126911 |

TABLE A7-2
Time-point: 7
Layer: 1606 (see FIG. 16)

| Neuron | biasₒᵤₜₚᵤₜ |
|---|---|
| output | −0.334037 |

TABLE A7-3
Time-point: 7
Layer: 1604 (See FIG. 16)

| | |
|---|---|
| Number of neurons | 10 |

TABLE A7-4
Time-point: 7
Layer: 1606 (see FIG. 16)

| | |
|---|---|
| Number of neurons | 1 |

TABLE A7-5
Time-point: 7
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | −0.0509443 | 0.00700789 | 0.0202592 | −0.0423502 | −0.0435313 | −0.00875363 |
| j = 2 | 0.383563 | 0.27979 | −0.00508145 | −0.117923 | −0.0954717 | −0.167596 |
| j = 3 | 0.128043 | −0.0180976 | −0.0108961 | 0.0635902 | 0.00866126 | 0.0220896 |
| j = 4 | −0.0185134 | −0.0552326 | 0.002105 | −0.0266165 | 0.0243037 | 0.00321537 |
| j = 5 | −0.00968643 | −0.00968604 | −0.00169778 | −0.0643853 | 0.00328421 | −0.0362618 |
| j = 6 | −0.338278 | −0.0662815 | −0.0108147 | 0.232628 | 0.0465558 | 0.154689 |
| j = 7 | −0.166233 | −0.00426825 | −0.0138852 | 0.0639276 | −0.127036 | 0.0328389 |
| j = 8 | 0.0240533 | 0.0192517 | −0.0557709 | −0.0394794 | −0.0424592 | −0.0444259 |
| j = 9 | 0.0333794 | −0.0322385 | −0.0365956 | 0.00777515 | 0.0548657 | −0.0206536 |
| j = 10 | 0.053659 | −0.0516502 | 0.00747946 | 0.0570349 | 0.0101936 | 0.0524872 |
| | Neuron Weight | | | | | |
| | i = 7 | i = 8 | i = 9 | i = 10 | i = 11 | i = 12 |
| j = 1 | 0.0383361 | −0.0400319 | 0.0119827 | 0.0355864 | −0.0540918 | 0.00534198 |
| j = 2 | −0.273856 | 0.108923 | −0.0995305 | 0.116769 | 0.0316872 | 0.144118 |
| j = 3 | 0.00537073 | 0.0689499 | −0.0534562 | 0.0819946 | 0.0629925 | 0.0145156 |
| j = 4 | −0.0298976 | 0.0276198 | −0.043237 | −0.023305 | −0.0409293 | 0.0254082 |
| j = 5 | 0.0422508 | −0.0235052 | 0.0548225 | −0.0507929 | 0.00882117 | −0.0119055 |
| j = 6 | −0.180842 | 0.0935007 | 0.0691962 | 0.040806 | −0.0522967 | −0.00648796 |
| j = 7 | −0.05795 | 0.0307767 | 0.0746283 | 0.188816 | −0.00776269 | −0.119285 |
| j = 8 | −0.00114259 | −0.00454884 | −0.0899848 | 0.0561499 | −0.00316566 | 0.00414463 |
| j = 9 | −0.0238226 | 0.0574785 | −0.0560749 | −0.0242776 | 0.00159179 | 0.0404429 |
| j = 10 | −0.0834241 | 0.0895825 | −0.097184 | −0.00605768 | 0.00387184 | 0.0993683 |
| | Neuron Weight | | | | | |
| | i = 13 | i = 14 | i = 15 | i = 16 | i = 17 | i = 18 |
| j = 1 | 0.0388676 | 0.0738552 | 0.0461344 | 0.050767 | 0.0810171 | 0.00101267 |
| j = 2 | −0.0638298 | −0.0424777 | −0.175594 | −0.320571 | 0.350266 | −0.360956 |
| j = 3 | −0.0192968 | −0.0324721 | −0.05719 | −0.124685 | −0.0560079 | −0.00195061 |
| j = 4 | 0.00297519 | −0.0445468 | −0.0105388 | −0.0782829 | −0.000130819 | −0.0197089 |
| j = 5 | −0.0220505 | 0.0602589 | −0.0277237 | 0.0677382 | 0.0587364 | −0.0337271 |
| j = 6 | 0.11653 | 0.190747 | −0.0899615 | 0.00878697 | −0.20657 | 0.257076 |
| j = 7 | 0.00769511 | 0.16442 | −0.14587 | 0.107999 | −0.14705 | 0.104257 |
| j = 8 | 0.0306166 | 0.0062355 | 0.0150554 | −0.0456873 | 0.000996432 | −0.00946799 |
| j = 9 | 0.0105834 | −0.03312 | 0.0146548 | −0.0595946 | 0.0173352 | 0.00436892 |
| j = 10 | 0.0591688 | 0.0172436 | −0.00489133 | −0.0956903 | −0.0389836 | 0.0313724 |
| | Neuron Weight | | | | | |
| | i = 19 | i = 20 | i = 21 | i = 22 | i = 23 | i = 24 |
| j = 1 | 0.0313117 | −0.0340487 | −0.0514795 | 0.0777003 | −0.0109209 | 0.00371884 |
| j = 2 | 0.274606 | −0.208732 | −0.233562 | 0.0388583 | 0.0401101 | 0.125597 |
| j = 3 | −0.0407859 | 0.00638125 | 0.0939594 | −0.0647613 | −0.0744658 | −0.0463073 |
| j = 4 | 0.0102438 | 0.0350887 | 0.0116578 | −0.0746492 | −0.0310018 | −0.00327027 |
| j = 5 | 0.0611502 | −0.0698096 | −0.0245249 | 0.0356951 | 0.0141353 | 0.0157712 |
| j = 6 | −0.0613096 | 0.00227163 | 0.0878972 | 0.0289549 | 0.0465659 | 0.016273 |
| j = 7 | 0.0606797 | −0.0378386 | −0.0820875 | 0.161595 | 0.105204 | 0.0695389 |
| j = 8 | −0.0461877 | −0.018913 | 0.00931436 | −0.0497035 | 0.00499761 | −0.00858879 |
| j = 9 | −0.0511292 | 0.0730508 | 0.0885574 | −0.0408044 | −0.0324696 | −0.0553347 |
| j = 10 | −0.0698708 | 0.0879906 | 0.0868179 | −0.126607 | −0.0372143 | −0.0707016 |

TABLE A7-6
Time-point: 7
Layer: 1606 (see FIG. 16)

Neuron Weight

| | j = 1 | j = 2 | j = 3 | j = 4 | j = 5 |
|---|---|---|---|---|---|
| output | −0.143632 | 0.477039 | 0.226596 | 0.0901068 | −0.133214 |

Neuron Weight

| | j = 6 | j = 7 | j = 8 | j = 9 | j = 10 |
|---|---|---|---|---|---|
| output | −0.431202 | −0.424275 | 0.0616729 | 0.12488 | 0.23921 |

TABLE A8-1
Time-point: 8
Layer: 1604 (See FIG. 16)

| Neuron | Bias (bⱼ) |
|---|---|
| j = 1 | −0.0320614 |
| j = 2 | −0.0279102 |
| j = 3 | −0.00446489 |
| j = 4 | 0.0175012 |
| j = 5 | 0.109843 |
| j = 6 | 0.0495395 |
| j = 7 | −0.0129817 |
| j = 8 | −0.0156038 |
| j = 9 | −1.04123 |
| j = 10 | −0.0241984 |

TABLE A8-2
Time-point: 8
Layer: 1606 (see FIG. 16)

| Neuron | biasₒᵤₜₚᵤₜ |
|---|---|
| Output | −0.533107 |

TABLE A8-3
Time-point: 8
Layer: 1604 (See FIG. 16)

| | |
|---|---|
| Number of neurons | 10 |

TABLE A8-4
Time-point: 8
Layer: 1606 (see FIG. 16)

| | |
|---|---|
| Number of neurons | 1 |

TABLE A8-5
Time-point: 8
Layer: 1604 (See FIG. 16)

| | Neuron Weight | | | | | |
|---|---|---|---|---|---|---|
| | i = 1 | i = 2 | i = 3 | i = 4 | i = 5 | i = 6 |
| j = 1 | 0.0129639 | −0.00919588 | −0.00379422 | −0.0341202 | −0.050187 | 0.0150229 |
| j = 2 | 0.0455819 | −0.0579266 | −0.0101471 | −0.0115712 | 0.00562151 | −0.0336361 |
| j = 3 | 0.0339524 | −0.0116563 | −0.1423 | 0.0734315 | 0.0575784 | 0.027224 |
| j = 4 | 0.018993 | 0.0383274 | 0.00280929 | −0.0102405 | 0.024326 | 0.0155249 |
| j = 5 | −0.0231274 | 0.0568002 | 0.00426666 | 0.0240056 | 0.00212137 | 0.0125846 |
| j = 6 | −0.0294393 | −0.00987101 | −0.0339821 | −0.00217749 | 0.0325147 | 0.0150087 |
| j = 7 | 0.0204672 | −0.0309236 | −0.0123943 | −0.0203132 | −0.0308943 | −0.0029819 |
| j = 8 | −0.155144 | 0.0654661 | 0.0424393 | 0.073026 | −0.00877629 | 0.0475555 |
| j = 9 | −0.488536 | −0.258248 | −0.236628 | 0.176356 | 0.0611459 | 0.173153 |
| j = 10 | 0.0744386 | −0.00860844 | −0.0948804 | −0.0614691 | −0.0088106 | 0.0307967 |
| | Neuron Weight | | | | | |
| | i = 7 | i = 8 | i = 9 | i = 10 | i = 11 | i = 12 |
| j = 1 | 0.010561 | 0.0694939 | −0.00603951 | −0.0313226 | 0.0122438 | 0.00450527 |
| j = 2 | 0.00704308 | 0.0323056 | −0.031571 | 0.00592607 | 0.0319889 | 0.0376764 |
| j = 3 | 0.00748841 | 0.071513 | −0.0624103 | −0.0236389 | 0.0153052 | 0.0082091 |
| j = 4 | −0.0013291 | −0.00777177 | −0.00413565 | −0.0306329 | 0.00372909 | −0.00155382 |
| j = 5 | −0.0750577 | −0.0717057 | 0.0421504 | 0.0114426 | −0.0237409 | −0.00723619 |
| j = 6 | −0.0379767 | 0.00526702 | 0.000514118 | 0.00636616 | 0.0205365 | 0.00237969 |
| j = 7 | 0.00980226 | 0.0511089 | −0.0293203 | −0.0197892 | 0.0400326 | −0.00807054 |
| j = 8 | −0.0360385 | −0.0861272 | 0.0617406 | 0.0748379 | −0.0394161 | −0.0140689 |
| j = 9 | 0.184257 | 0.00231628 | 0.169698 | −0.0132536 | −0.114835 | −0.353482 |
| j = 10 | −0.0302559 | 0.00585383 | −0.00183907 | −0.063558 | 0.00704066 | −0.00669166 |
| | Neuron Weight | | | | | |
| | i = 13 | i = 14 | i = 15 | i = 16 | i = 17 | i = 18 |
| j = 1 | 0.00411282 | −0.0952435 | 0.0100379 | 0.00570225 | 0.0096932 | 0.0621425 |
| j = 2 | −0.078058 | −0.0463274 | 0.0110102 | −0.0516843 | 0.00903558 | 0.0496182 |
| j = 3 | −0.0484272 | −0.02896 | 0.0110499 | −0.16845 | −0.0122031 | 0.0312596 |
| j = 4 | −0.0379691 | −0.0604454 | 0.00527064 | 0.00244192 | 0.069439 | 0.000990312 |
| j = 5 | 0.0601894 | 0.0212803 | 0.0512602 | −0.00811041 | 0.0699167 | −0.0258933 |
| j = 6 | −0.01584 | −0.0346578 | −0.0275457 | −0.00195105 | 0.0633908 | 0.0254076 |
| j = 7 | −0.0384226 | −0.0955863 | −0.0663125 | −0.0695451 | 0.0134783 | 0.0724134 |
| j = 8 | 0.088171 | 0.126874 | −0.00673456 | 0.0338833 | −0.00588901 | −0.0100279 |
| j = 9 | −0.101192 | 0.16946 | −0.186804 | 0.261064 | −0.44197 | 0.445205 |
| j = 10 | −0.0434092 | −0.0724122 | −0.0232755 | −0.0610583 | −0.0267507 | −0.031285 |
| | Neuron Weight | | | | | |
| | i = 19 | i = 20 | i = 21 | i = 22 | i = 23 | i = 24 |
| j = 1 | 0.00626678 | 0.0224523 | −0.0409126 | −0.00161839 | 0.0280284 | −0.0438442 |
| j = 2 | −0.0147576 | 0.0183857 | 0.0195045 | 0.0355948 | −0.0739363 | −0.0548402 |
| j = 3 | −0.127671 | 0.146875 | 0.0769104 | 0.00850982 | −0.180101 | −0.163133 |
| j = 4 | 0.0193799 | 0.0204076 | 0.0541138 | −0.0113546 | −0.0343254 | 0.0167634 |
| j = 5 | 0.0387368 | 0.0492472 | −0.0366052 | −0.0424456 | −0.0282696 | 0.00999604 |
| j = 6 | −0.0119244 | −0.028729 | −0.00383435 | −0.00277741 | −0.0518302 | 0.0322413 |
| j = 7 | −0.0159888 | −0.00866967 | −0.023676 | 0.00596156 | 0.0264748 | −0.0469121 |
| j = 8 | −0.0539799 | 0.116339 | 0.0965412 | −0.10139 | −0.00683711 | 0.0205406 |
| j = 9 | −0.0419999 | 0.0650154 | 0.0281133 | 0.33882 | 0.13654 | −0.123795 |
| j = 10 | 0.0776184 | −0.0200623 | −0.0464296 | 0.0933935 | 0.0202333 | 0.0085404 |

TABLE A8-6
Time-point: 8
Layer: 1606 (see FIG. 16)

| | Neuron Weight | | | | |
|---|---|---|---|---|---|
| | j = 1 | j = 2 | j = 3 | j = 4 | j = 5 |
| output | 0.0915148 | 0.0986086 | 0.239998 | −0.00725616 | −0.165409 |
| | Neuron Weight | | | | |
| | j = 6 | j = 7 | j = 8 | j = 9 | j = 10 |
| output | −0.019564 | 0.12 | −0.255865 | −0.817026 | 0.151464 |

What is claimed is:

1. A formulation comprising quetiapine or a pharmaceutically acceptable salt thereof wherein the quetiapine content is about 9.6% to about 10.4% by weight and wherein the formulation comprises about 30% hydroxypropyl methylcellulose by weight and about 7.2% sodium citrate dihydrate by weight.

2. The formulation of claim 1 wherein the quetiapine content is about 49.5 to about 50.5 mg.

3. The formulation of claim 2 comprising 30.0% hydroxypropyl methylcellulose by weight.

4. The formulation of claim 3 wherein:
about 15 to about 29 of the 30.0% hydroxypropyl methylcellulose is a first hydroxypropyl methylcellulose constituent;
the remainder of the 30.0% is a second hydroxypropyl methylcellulose constituent; and
the first and second constituents correspond, respectively, to a first hydroxypropyl methylcellulose grade that has an apparent viscosity between about 80 cp and about 120 cp and a second hydroxypropyl methylcellulose that has an apparent viscosity between about 3000 cp and about 5600 cp.

5. The form of claim 4 further comprising:
about 25.1 lactose monohydrate by weight;
about 25.1% microcrystalline cellulose by weight; and
about 1% magnesium stearate by weight.

6. A formulation comprising quetiapine or a pharmaceutically acceptable salt thereof wherein the quetiapine content is about 25.6 to about 26.5% by weight and wherein the dosage form comprises about 30% hydroxypropyl methylcellulose by weight and about 12.5% sodium citrate dihydrate by weight.

7. The formulation of claim 6 wherein the quetiapine content is about 149.5 to about 150.5 mg.

8. The formulation of claim 7 comprising 30.0% hydroxypropyl methylcellulose by weight.

9. The formulation of claim 8 wherein:
about 15 to about 29 of the 30.0% hydroxypropyl methylcellulose is a first hydroxypropyl methylcellulose constituent;
the remainder of the 30.0% is a second hydroxypropyl methylcellulose constituent; and
the first and second constituents correspond, respectively, to a first hydroxypropyl methylcellulose grade that has an apparent viscosity between about 80 cp and about 120 cp and a second hydroxypropyl methylcellulose that has an apparent viscosity between about 3000 cp and about 5600 cp.

10. The form of claim 8 further comprising:
about 13.0 lactose monohydrate by weight;
about 13.0% microcrystalline cellulose by weight; and
about 1.5% magnesium stearate by weight.

11. A formulation comprising quetiapine or a pharmaceutically acceptable salt thereof wherein the quetiapine content is about 32.9% to about 33.8% by weight and wherein the dosage form comprises about 12.5% sodium citrate dihydrate by weight and about 30% hydroxypropyl methylcellulose by weight.

12. The formulation of claim 11 wherein the quetiapine content is about 199.5 to about 200.5 mg.

13. The formulation of claim 12 comprising 30.0% hydroxypropyl methylcellulose by weight.

14. The formulation of claim 13 wherein:
about 15 to about 29 of the 30.0% hydroxypropyl methylcellulose is a first hydroxypropyl methylcellulose constituent;
the remainder of the 30.0% is a second hydroxypropyl methylcellulose constituent; and
the first and second constituents correspond, respectively, to a first hydroxypropyl methylcellulose grade that has a apparent viscosity between about 80 cp and about 120 cp and a second hydroxypropyl methylcellulose that has an apparent viscosity between about 3000 cp and about 5600 cp.

15. The formulation of claim 11 further comprising:
about 8.8% lactose monohydrate by weight;
about 8.8% microcrystalline cellulose by weight; and
about 1.5% magnesium stearate by weight.

16. A formulation comprising quetiapine or a pharmaceutically acceptable salt thereof wherein the quetiapine content is about 37.1% to about 38.0% by weight and wherein the dosage form comprises about 12.5% sodium citrate dihydrate by weight and about 30% hydroxypropyl methylcellulose by weight and wherein about 15 to about 29 of the 30% hydroxypropyl methylcellulose is a first hydroxypropyl methylcellulose constituent; the remainder of the 30% is a second hydroxypropyl methylcellulose constituent; and the first and second constituents correspond, respectively, to a first hydroxypropyl methylcellulose grade that has a apparent viscosity between about 80 cp and about 120 cp and a second hydroxypropyl methylcellulose that has an apparent viscosity between about 3000 cp and about 5600 cp,wherein the ratio of the first hydroxypropyl methylcellulose grade to the second hydroxypropyl methylcellulose grade is not 25.0 to 5.0.

17. A formulation comprising quetiapine or a pharmaceutically acceptable salt thereof wherein the quetiapine content is about 45.5% to about 46.4% by weight and wherein the dosage form comprises about 11.5% sodium citrate dihydrate by weight and about 30% hydroxypropyl methylcellulose by weight.

18. The formulation of claim 17 wherein the quetiapine content is about 399.5 to about 400.5 mg

19. The formulation of claim 18 comprising 30.0% hydroxypropyl methylcellulose by weight.

20. The formulation of claim 19 wherein:
about 15 to about 29 of the 30.0% hydroxypropyl methylcellulose is a first hydroxypropyl methylcellulose constituent;
the remainder of the 30.0% is a second hydroxypropyl methylcellulose constituent; and
the first and second constituents correspond, respectively, to a first hydroxypropyl methylcellulose grade that has a apparent viscosity between about 80 cp and about 120 cp and a second hydroxypropyl methylcellulose that has an apparent viscosity between about 3000 cp and about 5600 cp.

21. The formulation of claim 17 further comprising:
about 1.8% lactose monohydrate by weight;
about 1.8% microcrystalline cellulose by weight; and
about 2.0% magnesium stearate by weight.

22. A formulation of any one of claims 1, 6, 11, 16, 17 that satisfies the following dissolution criteria, when dissolution takes place in a basket apparatus having a rotation speed of 200 revolutions per minute and containing 900 milliliter 0.05 molar sodium citrate and 0.09 molar sodium hydroxide, to which 100 milliliter 0.05 molar sodium phosphate and 0.46 molar sodium hydroxide are added after 5 hours:
during the first 1-hour period of the dissolution, no more than 20% of the quetiapine is dissolved;
during the first 6-hour period of the dissolution, 47-69% of the quetiapine is dissolved;
during the first 12-hour period of the dissolution, 65-95% of the quetiapine is dissolved;
during the first 20-hour period of the dissolution, at least 85% of the quetiapine is dissolved.

23. A method of effectively treating psychoses in humans, comprising orally administering to a human patient on a once-a-day basis an oral extended release dosage form containing quetiapine or a pharmaceutically acceptable salt thereof wherein the quetiapine content is 50 mg which at steady-state provides a time to maximum plasma concentration (tₘₐₓ) of said antipsychotic in about 2 to about 16 hours, a maximum plasma concentration (Cₘₐₓ) which is greater than or equal to four times the plasma concentration of said antipsychotic at about 24 hours, and which dosage form provides effective treatment of psychoses for about 24 hours or more after administration to the patient.

24. A method of effectively treating psychoses in humans, comprising orally administering to a human patient on a once-a-day basis an oral extended release dosage form containing quetiapine or a pharmaceutically acceptable salt thereof wherein the quetiapine content is 150 mg which at steady-state provides a time to maximum plasma concentration (tₘₐₓ) of said antipsychotic in about 2 to about 16 hours, a maximum plasma concentration (Cₘₐₓ) which is greater than or equal to four times the plasma concentration of said antipsychotic at about 24 hours, and which dosage form provides effective treatment of psychoses for about 24 hours or more after administration to the patient.

25. A method of effectively treating psychoses in humans, comprising orally administering to a human patient on a once-a-day basis an oral extended release dosage form containing quetiapine or a pharmaceutically acceptable salt thereof wherein the quetiapine content is 200 mg which at steady-state provides a time to maximum plasma concentration (tₘₐₓ) of said antipsychotic in about 2 to about 8 hours, a maximum plasma concentration (Cₘₐₓ) which is greater than or equal to four times the plasma concentration of said antipsychotic at about 24 hours, and which dosage form provides effective treatment of psychoses for about 24 hours or more after administration to the patient.

26. A method of effectively treating psychoses in humans, comprising orally administering to a human patient on a once-a-day basis an oral extended release dosage form containing quetiapine or a pharmaceutically acceptable salt thereof wherein the quetiapine content is 400 mg which at steady-state provides a time to maximum plasma concentration (tₘₐₓ) of said antipsychotic in about 3 to about 8 hours, a maximum plasma concentration (Cₘₐₓ) which is greater than or equal to four times the plasma concentration of said antipsychotic at about 24 hours, and an area under curve between the time of administration and 24 hours after administration (AUC_{cum, 24}) which is greater than or equal to about 6000 ng.hr/mL, and which dosage form provides effective treatment of psychoses for about 24 hours or more after administration to the patient.
